(19) United States
(12) Patent Application Publication
(10) Pub. No.: US 2024/0425914 A1
(43) Pub. Date: Dec. 26, 2024

(54) EPITRANSCRIPTOME EVALUATION

(71) Applicant: HTG Molecular Diagnostics, Inc., 3430 E. Global Loop Tucson, AZ (US)

(72) Inventor: Debrah Thompson, Tucson, AZ (US)

(73) Assignee: HTG Molecular Diagnostics, Inc., 3430 E. Global Loop Tucson, AZ (US)

(21) Appl. No.: 18/687,476

(22) PCT Filed: Aug. 29, 2022
(86) PCT No.: PCT/US2022/075590

§ 371 Date: Feb. 28, 2024

Related U.S. Application Data
(60) Provisional application No. 63/239,703, filed on Sep. 1, 2021.

(51) Int. Cl. C12Q 1/6869; C12Q 1/6804; C12Q 1/6876

(52) U.S. Cl. CPC C12Q 1/6869; C12Q 1/6804; C12Q 1/6876

(57) ABSTRACT

The present disclosure provides methods of using quantitative nuclease protection sequencing (qNPS) methods to evaluate epitranscriptomes. The disclosed methods capture nucleic acid molecules that are modified as a result of exposure to an agent, such as stress, a mutagen, a microbe (such as a pathogen), or therapeutic agent. The resulting modified nucleic acid molecules are then directly or indirectly sequenced using nuclease protection probes (NPPs) and qNPS, allowing for the detection of target and off-target modifications of an epitranscriptome.

### CROSS-REFERENCE TO RELATED APPLICATION

This application claims priority to U.S. provisional Application No. 63/239,703 filed Sep. 1, 2021, herein incorporated by reference in its entirety.

### FIELD

The present disclosure provides methods for analyzing epitranscriptomes, such as RNA modifications that result from exposure to an agent (e.g., therapeutic agent, microbe, mutagen, stress such as heat shock, etc.). Such methods identify on-target and off-target effects on the epitranscriptome by the agent.

### BACKGROUND

Although methods of detecting and sequencing nucleic acid molecules are known, there is still a need for methods that permit analysis of multiple samples or multiple sequences simultaneously or contemporaneously. Methods of multiplexing nucleic acid molecule detection or sequencing reactions have not been realized at the most desired performance or simplicity levels. Furthermore, methods are needed to identify off-target effects resulting from therapeutic reagents, such as those that modify RNAs in locations outside of the target or that modify non-target RNAs.

### SUMMARY

Methods are disclosed herein for the capture, enrichment, and subsequent sequencing of modified nucleic acids. The reliability and versatility of these methods make them applicable for use in a wide range of fields. It has been observed that differing epitranscriptomic profiles, or variations in nucleic acid modifications, can have significant impacts on an organism's viability and phenotype. The methods disclosed herein provide a means to evaluate epitranscriptome and nucleic acid modifications, for example to examine an organism's growth, development, response to drug treatment, and disease susceptibility. For example, the disclosed methods can be used to identify and quantify epitranscriptomic effects of agents (e.g., therapeutic agents such as pharmaceuticals, mutagens, pathogens, stress, and the like). In one example, the methods are used in drug testing studies to identify on-target and off-target effects of drugs on the epitranscriptome of treated cells.

In particular examples, the disclosed methods utilize an antibody (or other specific binding agent) to recognize and reversibly bind to a specific chemical modification on a nucleic acid. For example, a N⁶-methyladenosine modification on an RNA molecule can be recognized and reversibly bound by an anti-N⁶-methyladenosine antibody. The antibody capturing the modified nucleic acid can be optionally bound to a solid support, from which the bound modified nucleic acids can optionally be released (e.g., by using proteinase K or heating in lysis buffer). This enriched or purified modified nucleic acid population can then be further processed and analyzed with sequencing methods utilizing nuclease protection probes that include one or more flanking sequences (NPPFs). The NPPFs each include a sequence that is complementary to all or a portion of a modified nucleic acid molecule, thus permitting specific binding or hybridization between the modified nucleic acid molecule and the NPPF. For example, the region of the NPPF that is complementary to a region of the modified nucleic acid molecule binds to or hybridizes to that region of the modified nucleic acid molecule with high specificity. The NPPFs further include one or more flanking sequences at the 5′-end and/or 3′-end of the NPPF. Thus, the one or more flanking sequences are located 5′, 3′, or both, to the sequence complementary to the modified nucleic acid molecule. If the NPPF includes a flanking sequence at both the 5′-end and 3′-end, in some examples the sequence of each NPPF is unique and not complementary to the nucleic acid molecule whose sequence is to be determined. The flanking sequence(s) includes several contiguous nucleotides having a sequence (such as a sequence of at least 12 nucleotides) not found in a nucleic acid molecule present in the sample and provide a universal hybridization and/or amplification sequence. This universal hybridization and/or amplification sequence, when having a sequence complementary to at least a portion of an amplification primer, permits multiplexing, as the same amplification primers can be used to amplify NPPFs specific for different modified nucleic acid molecules. It also provides a universal hybridization sequence for all NPPFs, which can be used to add a detectable label to the NPPF or to capture and concentrate NPPFs. For example, if the same flanking sequence is present on NPPFs specific for different modified nucleic acid molecules, the same primer can be used to amplify any NPPF having the same flanking sequence, even if the NPPF targets a different modified nucleic acid molecule. For example, the flanking sequence can be used to capture NPPFs, such as onto a surface. The flanking sequence can contain a variable sequence, such as a sequence that is specific for each specific NPPF and can be used to either capture that NPPF on a surface or for other purposes, such as to identify the NPPF. Thus, in some examples, the disclosed methods are used to sequence several different modified nucleic acid molecules (such as a plurality of modified nucleic acid molecules) in a sample using a plurality of NPPFs, wherein each NPPF specifically binds to a particular modified nucleic acid molecule. In one example, the disclosed methods sequence at least one modified nucleic acid molecule in a plurality of samples simultaneously. In one example, the disclosed methods sequence at least 1000, at least 10,000, or at least 20,000 different modified nucleic acid molecules in a plurality of samples simultaneously, for example by using at least 1000, at least 10,000, or at least 20,000 different NPPFs, respectively.

The disclosure provides methods for detecting or determining the sequence of one or more modified nucleic acid molecules in a sample (e.g., a cell lysate). In one example, the disclosed methods determine the sequence at least 1000, at least 2000, at least 3000, at least 4000, at least 5000, at least 10,000, or at least 20,000 different/unique modified nucleic acid molecules in a sample. In some examples, the sample is a cell lysate, such as one generated from a blood sample, cell culture, FFPE sample, or tissue biopsy. The methods can include contacting the sample (such as a cell lysate) containing a complex mixture of modified and unmodified nucleic acids with an antibody specifically recognizing a particular nucleic acid modification. Thus, only modified nucleic acid molecules will bind to the antibody, and unmodified nucleic acid molecules can be separated away (e.g., by washing). In some examples, the antibody is attached to a solid support (such as beads). The methods can optionally further include releasing the modified nucleic acids from the antibody or solid support (e.g., treatment with buffer containing SDS and/or formamide (e.g., a lysis buffer) and heating at about 95° C. for about 10-15 minutes to release modified nucleic acids from the antibody, treatment with proteinase K to digest the antibody thereby releasing the modified nucleic acids), thereby producing purified modified nucleic acids. However, in some examples, the solid support containing the antibody and modified nucleic acids is used without a separate separation step. In some examples, the purified modified nucleic acids obtained are further purified, for example with a commercially available DNA or RNA isolation kit, such as guanidinium or phenol protocols. The methods can subsequently include contacting the purified modified nucleic acids (which may or may not be attached to the antibody and solid support) with at least one NPPF under conditions sufficient for the NPPF to specifically bind to a nucleic acid molecule containing the modification. The NPPF molecule includes a sequence complementary to all or a portion of a modified nucleic acid molecule. This permits specific binding or hybridization between the NPPF and the modified nucleic acid molecule. Thus, by using a plurality of NPPFs, permits sequencing of a plurality of modified nucleic acid molecules. In some examples, at least 1000, at least 10,000, at least 20,000, at least 30,000, at least 50,000, or at least 100,000 unique NPPFs are used in a single reaction. The method further includes contacting the sample with one or more nucleic acid molecules having a sequence that is complementary to all or a portion of a flanking sequence (such a molecule is referred to herein as a CFS) under conditions sufficient for the flanking sequence to specifically bind or hybridize to the CFS. More than one CFS can be used to hybridize to an entire flanking sequence (e.g., multiple individual CFSs can be hybridized to a single flanking sequence, such that the entire flaking sequence is covered). This generates NPPF molecules that have bound (hybridized) thereto modified nucleic acid molecule, as well as the CFS(s), thereby generating a double-stranded molecule, which can include at least four contiguous oligonucleotide sequences, in which all bases can be engaged in hybridization to a complementary base (though 100% complementarity is not required, for example at least 80%, at least 90%, or at least 95% complementarity can be sufficient).

After allowing the modified nucleic acid molecules and the CFS(s) to bind to the NPPFs, the method can further include contacting the sample with a nuclease specific for single-stranded (ss) nucleic acid molecules (or ss regions of a nucleic acid molecule) under conditions sufficient to remove nucleic acid bases that are not hybridized to a complementary base. Thus, for example, NPPFs that have not bound modified nucleic acid molecules or CFSs, as well as unbound modified nucleic acid molecules, other ss nucleic acid molecules in the reaction, and unbound CFSs, will be degraded. This generates a digested sample that includes intact NPPFs present as double-stranded adducts with CFS(s) and modified nucleic acid molecule(s). In some examples, the method further includes increasing the pH of the sample and/or heating it, to dissociate or remove modified nucleic acid molecules and CFSs that are bound to the NPPFs.

The NPPFs that were bound to the modified nucleic acid molecule and CFSs, and thus survived treatment with the nuclease, can be amplified and/or labeled. NPPFs in the digested reaction can be amplified using one or more amplification primers, thereby generating NPPF amplicons. At least one amplification primer includes a region that is complementary to all or a portion of the flanking sequence of the NPPF. In some examples, the NPPF includes a flanking sequence at both the 5′-end and 3′-end, and two amplification primers are used, wherein one amplification primer has a region that is complementary to the 5′-end flanking sequence and the other amplification primer has a region that is complementary to the 3′-end flanking sequence.

Alternatively, instead of using the NPPFs that survived treatment with the nuclease, the modified nucleic acid strand that was hybridized to the NPPF (such as a DNA strand) can be used directly, such as amplified, labeled, detected, sequenced, or combinations thereof. For example, the modified nucleic acid strand can be amplified using one or more amplification primers, thereby generating modified nucleic acid molecule amplicons, which can be detected and/or sequenced. Thus, although NPPF amplicons are referred to herein, one will appreciate that modified nucleic acid molecule amplicons can be substituted therefor.

The resulting amplicons (or portion thereof, such as a 3′-portion) can then be sequenced or detected. In one example, amplicons are attached to a substrate. For example, the substrate can include at least one capture probe having a sequence complementary to all or a portion of a flanking sequence on the NPPF amplicon, thus permitting capture of the NPPF amplicons having the complementary flanking sequence. The captured NPPF amplicons can then be sequenced, thereby determining the sequence of modified nucleic acid molecules in the sample.

In other examples, the NPPF amplicons are sequenced without capture onto an array. For example, the NPPF amplicons can be transferred to a sequencing platform.

The NPPF can be labeled with a detectable label, for example during amplification, or as a step without amplification. Alternatively, one or both flanking regions can be used to hybridize a detectable label to the NPPF.

In some examples, a control sample is analyzed in parallel to the test sample, wherein the sequence of both modified and unmodified nucleic acids in the control sample are determined, for example to permit comparisons to the modified nucleic acid molecules identified in the test sample. In such a control sample, the modified nucleic acid molecules need not be first purified or isolated from the sample, but instead, the sample is contacted with a lysis buffer and the exposed nucleic acids in the control sample contacted with NPPFs and CFSs and heated (e.g., about 95° C. to about 105° C. for about 5-15 minutes) to denature nucleic acids in the control sample and incubated permit hybridization between the NPPFs, CFSs, and the nucleic acids in the control sample (e.g., about 10 minutes to about 72 hours, for example, at least about 1 hour to 48 hours, about 6 hours to 24 hours, about 12 hours to 18 hours, or overnight, at a temperature from about 4° C. to about 70° C., for example, about 37° C. to about 65° C., about 42° C. to about 60° C., or about 50° C. to about 60° C.). The procedure then follows the process shown in steps 2-5 in FIGS. 2 and 4.

The foregoing and other objects and features of the disclosure will become more apparent from the following detailed description, which proceeds with reference to the accompanying figures.

### BRIEF DESCRIPTION OF THE DRAWINGS

FIG. 1 is a schematic diagram showing an exemplary nuclease protection probe having flanking sequences (NPPF), 100. The NPPF 100 includes a region 102 having a sequence that specifically hybridizes to a nucleic acid sequence (e.g., shares complementarity to a modified or unmodified nucleic acid whose sequence is to be determined). The NPPF also includes a 5′-flanking sequence 104 and a 3′-flanking sequence 106.

FIG. 2A is a schematic diagram showing the steps of a method (e.g., step 5 in FIG. 9) of using the NPPFs 202 (202a, 202b) to sequence modified nucleic acid molecules 200 (e.g., 500 in step 4 of FIG. 9) after it has been isolated from a sample (e.g., see FIG. 9). The lighter solid bars 202a represent an NPPF specific for a first modified nucleic acid molecule, the dashed bars 202b represent an NPPF specific for a second modified nucleic acid molecule, the dotted bars 204 represent nucleic acid molecules that are complementary to the flanking sequences (CFS) 204 of the NPPF, and the solid bars represent modified nucleic acids 200 (e.g., DNA or RNA) (or portions thereof 200a). (1) modified nucleic acid molecules obtained from a sample (see FIG. 9) are incubated with the NPPFs and CFSs in the presence of a denaturation buffer (e.g., lysis buffer) with a denaturation step (e.g., about 95° C. to about 105° C. for about 5-15 minutes) followed by a hybridization step (e.g., incubation for about 10 minutes to about 72 hours, for example, at least about 1 hour to 48 hours, about 6 hours to 24 hours, about 12 hours to 18 hours, or overnight; at a temperature from about 4° C. to about 70° C., for example, about 37° C. to about 65° C., about 42° C. to about 60° C., or about 50° C. to about 60° C.). (2) Unbound (e.g., single-stranded) nucleic acid is digested with a nuclease specific for ss nucleic acid molecules (such as S1 nuclease). (3) The nuclease can be inactivated and the NPPFs dissociated from hybridized modified molecules and hybridized CFSs, for example by addition of base and heating. (4) The remaining NPPFs are amplified, for example by using PCR with appropriate primers 208. In some examples, the primers 208 include a detectable label (such as biotin or a fluorophore), to permit labeling of the resulting amplicons 210. The resulting amplicons 210 can be detected or sequenced (FIG. 2B), thus allowing for detection or sequencing of modified nucleic acid molecules 200.

FIG. 2B is a schematic diagram showing that NPPF amplicons 210 can be (5) sequenced, thereby determining the sequence of modified nucleic acids 200.

FIGS. 3A-3B are schematic diagrams showing details of the nucleic acid molecules as they are processed during the steps of a method of using the NPPFs 402 to sequence modified nucleic acid molecules using the disclosed methods. The longer solid colored bars 400a, 400b, 400c represent modified nucleic acid molecules 400, the bars with lighter and darker colors on their ends are NPPFs 402 specific for a modified nucleic acid molecules 400, with the different colored ends 404 representing the flanking sequences. The color of the modified nucleic acid molecule is matched to the color of its corresponding NPPF. The shorter solid bars represent nucleic acid molecules that are complementary to the flanking sequences (CFS) 406 of the NPPF.

FIGS. 4A-4F are schematic drawings showing exemplary embodiments of NPPF molecules, including embodiments with (A and B) a flanking sequence only on one end of the NPPF or (C-F) with flanking sequences on both ends of the NPPF.

FIGS. 5A and 5B are bar graphs showing the signal-to-noise ratio of immunoprecipitation, wash 1, and elution fractions of the sample, where the “signal” is modified RNAs containing N⁶-methyladenosine (m⁶A) and the noise is the unmodified RNAs in the fraction. The error bars are one standard deviation from the mean. Measurements by standard RT-qPCR are shown in (A) and measurements using HTG EdgeSeq are shown in (B) FIGS. 6A and 6B are two aspects of the results from enrichment of cellular RNAs where (A) is the correlation between replicate enrichment reactions, and (B) is the correlation between enrichment reactions and the parent, non-enriched sample.

FIGS. 7A and 7B are bar graphs demonstrating the efficacy of modified nucleic acid enrichment via comparison to external controls where (A) shows the raw counts, modified and unmodified IVTs and (B) shows the adjusted signal-to-noise ratio.

FIG. 8 is a scatterplot correlation between enriched and non-enriched parent samples showing a strong correlation between the two replicates of the non-enriched parent samples (Spearman correlation coefficient of 0.97), good correlation between two replicates of enrichment samples (Spearman correlation coefficient of 0.88), and poorer correlation between the two types of samples (Spearman correlation coefficient of 0.77).

FIG. 9 is an exemplary workflow schematic showing the immunocapture of modified nucleic acids 500 from a mixture (e.g., a cell lysate) that includes unmodified nucleic acids 501 and 502. At step 2, the sample is incubated with antibody 503, which is optionally attached to a solid support 504 (such as a bead). Antibody 503 selectively binds modified nucleic acids 500 present in the mixture containing unmodified nucleic acids 501, 502. In step 3, the unmodified nucleic acids 501, 502 are separated away from the captured modified nucleic acids 500, for example by washing. In optional step 4, modified nucleic acids 500 are released from the antibody 503 and solid support 504, for example by digesting antibodies 505 with proteinase K to release modified nucleic acids 500, by heating the reaction to about 95 C for 10-15 minutes in the presence of a lysis buffer (e.g., one that includes SDS and/or formamide), and the like. Optionally, modified nucleic acids 500 can be purified, for example using a nucleic acid purification kit, such as a DNA or RNA purification kit. In step 5, the separated (e.g., purified) modified nucleic acids 500 are sequenced using NPPFs (e.g., illustrated in FIGS. 2-4).

FIGS. 10A and 10B are two aspects of the results from enrichment of cellular RNAs where (A) enrichment is performed by different operators, and (B) enrichment is performed using an antibody different from the antibody used in the enrichments in (A).

FIG. 11 is a scatterplot correlation between samples having different sample inputs added to the immunoprecipitation reaction.

FIGS. 12A and 12B are two scatterplot correlations between samples for (A) pulldown samples and (B) parent samples.

FIGS. 13A and 13B are volcano plots of (A) differentially expressed genes and (B) m⁶A RNA modification changes resulting from heat shock treatment.

FIGS. 14A and 14B are graphs illustrating (A) RNA expression level and (B) m⁶A RNA modification changes resulting from treatment of cells with heat shock.

FIGS. 15A and 15B are PCA plots showing (A) m⁶A RNA modifications and (B) RNA expression profiles measured from samples of cells treated with agents or a control.

### SEQUENCE LISTING

The nucleic acid sequences listed herein are shown using standard letter abbreviations for nucleotide bases, as defined in 37 C.F.R. 1.822. Only one strand of each nucleic acid sequence is shown, but the complementary strand is understood as included by any reference to the displayed strand. The Sequence Listing is submitted as an ST.26 Sequence Listing XML file, named seq listing, created on Aug. 18, 2022, having a size of 4539 bytes, which is incorporated by reference herein. In the provided sequences:
SEQ ID NOS 1-4 provide exemplary primers for determining amounts of RNA in a sample using RT-qPCR.

### DETAILED DESCRIPTION

### I. Overview

The present disclosure provides improved methods of identifying on-target and off-target effects of agents that modify nucleic acid molecules. The methods enrich or purify modified nucleic acid molecules and include steps that allow the sequences of the nucleic acid molecules to be obtained. The methods can thus include treating or exposing a cell or organism to an agent (such as a mutagen, pathogen or other microbe, pharmaceutical agent (such as a therapeutic agent, such as an anti-cancer, anti-viral, anti-bacterial, anti-fungal, or anti-parasitic agent)), stress (such as heat shock), disease state, or other change in cellular status, and subsequently identify on-target effects of such, off-target effects of such, or both, such as off-target effects in response to treatment with pharmaceutical agents. The disclosed methods provide several improvements over currently available enrichment and sequencing methods. For example, because the methods require less processing of modified nucleic acid molecules, bias introduced by such processing can be reduced or eliminated. For example, current methods of analyzing modified RNA typically employ steps to isolate or extract all the RNA from a sample, fragment the RNA, subject it to RT-PCR, ligate the RNA, or combinations thereof. Thus, at least two RNA isolation steps are required; RNA isolation from the sample, and after enrichment for modified RNAs. RNA fragmentation prior to enrichment is also common. In contrast, the disclosed methods do not require isolation of all RNA from the sample (instead a cell lysate can be generated), and enriched RNAs need not be further purified (but can be further purified, if so desired). As a result, the disclosed methods permit one to analyze a range of sample types not previously amenable to sequencing. In addition, the disclosed methods result in less loss of modified nucleic acids from the sample, providing a more accurate result (e.g., when data are quantified). It also reduces enzyme bias and bias introduced by nucleic acid extraction. The disclosed methods also provide for targeted detection and sequencing of a desired modified nucleic acid molecule. This greatly simplifies data analysis. Current methods are challenged by the large amount of data generated, and the need for complicated bioinformatics. Identification of modifications within data adds another layer of complexity to post-sequencing data analysis. Although costs of sequencing have decreased, the ability to determine sequences is outrunning the ability of researchers to store, transmit and analyze the data. As a result, there is commonly more data generated than can be analyzed in a reasonable amount of time. Because the disclosed methods are targeted, it can overcome these obstacles. For example, the amount of data generated is simplified, as only a portion of a captured modified nucleic acid needs to be sequenced. Long reads of nucleotides are not required, nor do fragments of sequences need to be properly aligned to a reference sequence, or modified regions of the nucleic acid inferred from sequence changes or detection of enrichment at a site. In addition, the results can be simply counted, without the need for complicated bioinformatics analysis.

For example, the method can be used to enrich modified DNA or RNA, and then determine the sequence of the enriched modified nucleic acids (e.g., by directly sequencing the modified DNA or RNA, or indirectly by sequencing an NPPF or NPPF amplicon), to identify where modifications have occurred in DNA or RNA, for example in response to treatment with one or more test agents (e.g., pharmaceutical agents, microbes, mutagens, etc.) or test conditions (e.g., stress, such as heat shock or cold shock). For example, the methods can be used to determine if one or more test agents or conditions modify a nucleic acid where expected or desired, increase or decrease the level of modification of a target, and/or whether one or more test agents/conditions result in off-target modifications (which may be undesirable). The method uses an antibody that specifically binds to a modified nucleic acid molecule (such as N⁶-methyladenosine) under conditions that allow the antibody to bind to modified nucleic acid molecules in the sample (such as a cell lysate), thereby forming an antibody-nucleic acid conjugate. The antibody-nucleic acid conjugate is separated away from unbound, unmodified nucleic acids, thereby generating an enriched antibody-nucleic acid conjugate (e.g., by washing). Optionally, the antibody (and in some examples the solid support) can be separated away (e.g., removed) from the modified nucleic acid molecules (e.g., by heating the reaction in the presence of SDS and/or formamide (e.g., a lysis buffer), such as 90-100° C. for at least 5 minutes, such as at least 10 minutes, or at least 15 minutes, such as 95° C. for 10-15 minutes; or contacting the reaction with proteinase K to digest the antibody), thereby generating a mixture that includes the purified/enriched modified nucleic acid molecules. In some examples, the antibody-nucleic acid conjugate, which may be attached to a solid support (such as a bead) can be used directly, for example incubated with NPPFs and CSFs and lysis buffer and heat (the reaction is denatured, and then allowed to hybridize). In some examples, the sample is treated with a DNAase prior to contacting the sample with the antibody that specifically binds to a modified nucleic acid molecule.

The resulting modified nucleic acid molecules can then be directly or indirectly sequenced, for example to identify on-targeted or off targeted nucleic acid modifications. To sequence the purified or enriched one or more modified nucleic acid molecules, modified nucleic acid molecules are contacted with a probe, referred to herein as a nuclease protection probe comprising a flanking sequence (NPPF). The use of NPPFs permit multiplexing, because the flanking sequences on the probe permit universal primer binding sites for amplification and permit addition of sequencing adapters and experimental tags (at either the 3- or the 5′-end, or at both ends for example to increase multiplexing), without destroying the stoichiometry. As the flanking sites can be universal, the same primers can be used to amplify any NPPF for any modified sequence, thus allowing for multiplexing and conservation of stoichiometry. In one example, by amplifying from both ends of the NPPF, the methods provide greater specificity. Only NPPFs with intact 3- and 5′-flanking sequences will be amplified exponentially, while NPPFs cleaved by the nuclease will not be amplified sufficiently to be sequenced or detected.

In addition, the primers permit addition of tags (such as experiment tags to permit the identification of a particular modified nucleic acid molecule without necessitating the sequencing of the entire NPPF itself or to permit samples from different patients to be combined into a single run, at either the 3′- or the 5′-end, or at both ends for example to increase multiplexing, as well as sequencing adapters to permit attachment of a sequence needed for a particular sequencing platform and formation of colonies for some sequencing platforms). The use of NPPFs also simplifies the complexity of the sample that is analyzed (e.g., sequenced), as it reduces the sample containing, for example, modified nucleic acid molecules, to the NPPFs (or NPPF or target amplicons). The sequencing of NPPFs (or the modified nucleic acid molecule hybridized to the NPPF) simplifies data analysis compared to that required for other sequencing methods, reducing the algorithm to simply count the matches to the NPPFs that were added to the sample, rather than having to match modified nucleic acid molecule sequences to the genome and deconvolute the multiple sequences that are obtained from standard methods of sequencing. In some examples, the disclosed methods increase the signal obtained as compared to prior methods, such as an increase of at least 10-fold, at least 100-fold, at least 125-fold, at least 150-fold, or at least 200-fold without substantial dilution of the NPPF product before performing the amplification.

In one example, the disclosure provides methods for sequencing at least one modified nucleic acid molecule in a sample (such as at least 2, at least 3, at least 4, at least 5, at least 10, at least 20, at least 30, at least 40, at least 50, at least 100, at least 500, at least 1000, at least 2000, at least 3000, at least 4000, at least 5000, at least 10,000, at least 15,000, at least 20,000, at least 30,000, at least 40,000, at least 50,000, at least 75,000, or at least 100,000 modified nucleic acid molecules, such as different modified nucleic acid molecules and/or different locations within a single modified nucleic acid molecule). In some examples, the sample is a lysed biological sample, for example treated with a buffer that includes a denaturant, such as SDS, formamide, glyoxal, etc. In some examples, the sample is treated with DNAase to denature DNA molecules in the sample, for example when a modified RNA is to be identified and sequenced. In some examples, the sample is a fixed sample (such as a paraffin-embedded formalin-fixed (FFPE) sample, hematoxylin and eosin stained tissues, or glutaraldehyde fixed tissues), which can be lysed prior to its analysis. Thus, in some examples, the modified nucleic acid molecules to be identified can be fixed, cross-linked, or insoluble.

The methods can include contacting the modified nucleic acid molecules collected from the sample using an antibody specific for the modification, with at least one nuclease protection probe comprising a flanking sequence (NPPF) under conditions sufficient for the NPPF(s) to specifically bind to modified nucleic acid molecules. In some examples, the disclosed methods determine the sequence of one or more modified nucleic acid molecules in a plurality of samples simultaneously or contemporaneously. In some examples, the disclosed methods determine the sequence of two or more modified nucleic acid molecules in a sample (for example simultaneously or contemporaneously). In such an example, the sample is contacted with a plurality of NPPFs, wherein each NPPF specifically binds to a particular modified nucleic acid molecule (or portion of a modified nucleic acid molecule). For example, the sample can be contacted with 10 different NPPFs allowing the sequence of 10 modified nucleic acid molecules to be determined, wherein each NPPF is specific for one of the 10 modified nucleic acid molecules. However, it is appreciated that in some examples, more than one NPPF (such as 2, 3, 4, 5, 10, 20, or more) specific for a single modified nucleic acid molecule can be used, such as a population of NPPFs that are specific for different regions of a particular modified nucleic acid molecule, or a population of NPPFs that can bind to a modified nucleic acid molecule and variations thereof (such as those having mutations or polymorphisms). In some examples, at least 10,000 different NPPFs, such as at least 20,000, at least 50,000, or at least 100,000 different NPPFs are incubated with the modified nucleic acid molecules collected from the lysed sample, thereby allowing the sequence of at least at least 10,000 different modified nucleic acid molecules, at least 20,000, at least 50,000, or at least 100,000 different modified nucleic acid molecules, portions of modified nucleic acid molecules, or combinations thereof, respectively, to be determined.

The NPPF molecule includes a 5′-end and a 3′-end, as well as a sequence in between that is complementary to all or a part of a modified nucleic acid molecule. This permits specific binding or hybridization between the NPPF and a modified nucleic acid molecule. For example, the region of the NPPF that is complementary to a region of the modified nucleic acid molecule binds to or hybridizes to that region of the modified nucleic acid molecule with high specificity. The NPPF can be complementary to all of, or a portion of, the modified nucleic acid sequence. The NPPF molecule further includes one or more flanking sequences, which are at the 5′-end and/or 3′-end of the NPPF. Thus, the one or more flanking sequences are located 5′, 3′, or both, to the sequence complementary to the modified nucleic acid molecule. Each flanking sequence includes several contiguous nucleotides, generating a sequence that is not found in a nucleic acid molecule present in the sample (such as a sequence of at least 12 contiguous nucleotides). If the NPPF includes a flanking sequence at both the 5′-end and 3′-end, in some examples the sequence of each NPPF is unique.

The flanking sequence(s) provide a universal hybridization/amplification sequence, which is complementary to at least a portion of an amplification primer. In some examples, the flanking sequence can include (or permit addition of) an experimental tag, sequencing adapter, or combinations thereof. For example, the experimental tag can be a sequence complementary to a capture probe that permits capture NPPFs, for example onto a surface (such as at a specific spot on the surface, or to a specific bead). In some examples, the experimental tag can be a sequence that identifies an NPPF, such as a tag specific for a particular patient or modified sequence, for example to permit one to distinguish or group such tagged NPPFs. In some examples, the sequencing adapter can be a sequence that permits an NPPF amplicon to be used with a particular sequencing platform.

The NPPF can be any nucleic acid molecule, such as a DNA or RNA molecule, and can include unnatural bases. In some examples the NPPF is at least 35 nucleotides, such as 40 to 80 or 50 to 150 nucleotides. The portion of the NPPF that is complementary to a region of a modified nucleic acid molecule can be at least 6 nucleotides in length, such as at least 10, at least 25, or at least 60, such as 6 to 60 nucleotides in length. The flanking sequence(s) of the NPPF can be at least 6 nucleotides, at least 12 nucleotides, or at least 25 nucleotides, such as 12 to 50 nucleotides in length. In some examples, the NPPF includes two flanking sequences: one at the 5′-end and the other at the 3′-end. In some examples, the flanking sequence at the 5′-end differs from the flanking sequence at the 3′-end. In addition, if the NPPF includes two flanking sequences, ideally the two flanking sequences have a similar melting temperature (Tₘ), such as a Tₘ of +/−5° C.

The method further includes contacting the modified nucleic acid molecules collected from the sample using an antibody specific for the modification with a nucleic acid molecule having a sequence that is complementary to the flanking sequence (such a molecule is referred to herein as a CFS) under conditions sufficient for the flanking sequence to specifically bind or hybridize to the CFS. One skilled in the art will appreciate that instead of using a single CFS to protect a flanking sequence, multiple CFSs can be used to protect a flanking sequence. This results in the generation of NPPF molecules that have bound thereto a modified nucleic acid molecule, as well as the CFS, thereby generating a double-stranded molecule that includes at least four contiguous oligonucleotide sequences, with all bases engaged in hybridization to a complementary base i (though 100% complementarity is not required, for example at least 80%, at least 90%, or at least 95% complementarity can be sufficient), which bases of the NPPF and CFSs can include unnatural bases. The CFS hybridizes to and thus protects its corresponding flanking sequence from digestion with the nuclease in subsequent steps. In some examples, each CFS is the exact length of its corresponding flanking sequence. In some examples, the CFS is completely complementary to its corresponding flanking sequence. However, one skilled in the art will appreciate that the 3′-end of a CFS that protects a 5′-end flanking sequence or the 5′-end of a CFS that protects the 3′-end flanking sequence can have had a difference, such as one nucleotide at each of these positions.

After allowing a modified nucleic acid molecule, as well as the CFS(s), to bind to the NPPFs, the method can further include contacting the reaction with a nuclease specific for single-stranded (ss) nucleic acid molecules or ss regions of a nucleic acid molecule, such as S1 nuclease, under conditions sufficient to remove nucleic acid bases that are not hybridized to a complementary base. Thus, for example, NPPFs that have not bound a modified nucleic acid molecule or CFSs, as well as unbound modified nucleic acid molecules, other ss nucleic acid molecules in the reaction, and unbound CFSs, will be degraded. This generates a digested reaction that includes intact NPPFs present as double stranded adducts hybridized to CFSs and modified nucleic acid molecules. In some examples, for example if the NPPF is composed of DNA, the nuclease can include an exonuclease, an endonuclease, or a combination thereof.

In some examples, the method further includes increasing the pH of the reaction and/or heating it, for example to inactivate the nuclease, to remove modified nucleic acid molecule and CFSs that are bound to the NPPFs, or combinations thereof. In some examples, the method includes releasing the modified nucleic acid (such as a DNA) from the NPPF, and then further analyzing the released modified nucleic acid molecule (such as detecting or sequencing the modified nucleic acid molecule). In some examples the modified nucleic acid is DNA, and the DNA is amplified prior to its detection or sequencing.

The NPPFs that were bound to modified nucleic acid molecules and CFSs and thus survived treatment with the nuclease can be amplified, for example using PCR amplification. NPPFs in the digested reaction can be amplified using one or more amplification primers, thereby generating NPPF amplicons. At least one amplification primer includes a region that is complementary to an NPPF flanking sequence. In some examples, the NPPF includes a flanking sequence at both the 5′-end and 3′-end, and two amplification primers are used, wherein one amplification primer has a region that is complementary to the 5′-end flanking sequence and the other amplification primer has a region that is complementary to the 3′-end flanking sequence. One or both of the amplification primers can include a sequence that permits attachment of an experimental tag or sequencing adapter to the NPPF amplicon during the amplification, and one or both primers can be labeled to permit labeling of the NPPF amplicon. In some examples, both an experimental tag and a sequencing adapter are added, for example at opposite ends of the NPPF amplicon. For example, the use of such primers can generate an experimental tag or sequence tag extending from the 5′-end or 3′-end of the NPPF amplicon, or from both the 3′-end and 5′-end to increase the degree of multiplexing possible. The experimental tag can include a unique nucleic acid sequence that permits identification of a sample, subject, or modified nucleic acid sequence. In some examples, the amplification primer contains an experimental tag that permits capture of the NPPF amplicon onto a substrate (for example by hybridization to a probe on the substrate having a sequence complementary to the capture sequence on the NPPF amplicon). The sequencing adapter can include a nucleic acid sequence that permits capture of the resulting NPPF amplicon onto a sequencing platform. For example, the amplification primer can include a sequence that permits attachment of a poly-A or poly T sequence tag which can facilitate amplification once captured onto the sequencing chip. In some examples, the amplification primer is used to label the NPPF amplicon. In other examples, one or both flanking regions are used to hybridize a detectable label to the NPPF, such as with a labeled probe (for example without amplification).

The resulting NPPF (or modified nucleic acid molecule) amplicons (or portion thereof, such as a 3′-portion) can then be sequenced or detected, thereby determining the sequence of, or detecting, modified nucleic acid molecules in the sample.

In one example, the NPPF amplicons (or portion thereof) is sequenced. Any method can be used to sequence the NPPF amplicons, and the disclosure is not limited to particular sequencing methods. In some examples, the sequencing method used is Illumina sequencing, sequencing-by-synthesis, nanopore sequencing, sequencing-by-binding, semiconductor sequencing, nanopore sequencing, chain termination sequencing, dye termination sequencing, or pyrosequencing. In some examples, single molecule sequencing is used. In some examples where the NPPF amplicons are sequenced, the method also includes comparing the obtained NPPF sequence to a reference sequence database; and determining the number of each identified NPPF sequence.

### II. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

The following explanations of terms and methods are provided to better describe the present disclosure and to guide those of ordinary skill in the art to practice the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

All publications, patent applications, patents, and other references mentioned herein are incorporated by reference in their entirety for all purposes. All sequences associated with the GenBank® Accession Nos. mentioned herein are incorporated by reference in their entirety as were present on Sep. 1, 2021, to the extent permissible by applicable rules and/or law. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

To facilitate review of the various embodiments of this disclosure, the following explanations of specific terms are provided:

3′ end: The end of a nucleic acid molecule or sequence that does not have a nucleotide bound to it 3′ position of the terminal residue.

5′ end: The end of a nucleic acid molecule or sequence where the 5′ position of the terminal residue is not bound by a nucleotide.

Amplifying a nucleic acid molecule: To increase the number of copies of a nucleic acid molecule, such as an NPPF or portion thereof. The resulting products are called amplification products or amplicons. An example of in vitro amplification is the polymerase chain reaction (PCR), in which a sample (such as a sample containing NPPFs) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule.

Binding or stable binding (of a nucleic acid): A first nucleic acid molecule (such as an NPPF) binds or stably binds to another nucleic acid molecule (such as a modified nucleic acid molecule) if a sufficient amount of the first nucleic acid molecule forms base pairs or is hybridized to the other nucleic acid molecule, for example the binding of a NPPF to its complementary modified nucleic acid sequence.

Binding can be detected by either physical or functional properties. Binding between nucleic acid molecules can be detected, for example using functional (for example reduction in expression and/or activity) and/or physical binding assays.

Complementary: Ability to from base pairs between nucleic acids. Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between to distinct nucleic acids or two distinct regions of the same nucleic acid.

“Specifically hybridizable” and “specifically complementary” are terms that indicate a sufficient degree of complementarity such that stable and specific binding occurs between the probe (for example, an NPPF) or its analog and a modified nucleic acid (such as modified DNA or RNA). The probe or analog need not be 100% complementary to its target sequence to be specifically hybridizable. A probe or analog is specifically hybridizable when there is a sufficient degree of complementarity to avoid non-specific binding of the probe or analog to non-target sequences under conditions where specific binding is desired, for example in the methods disclosed herein.

Conditions sufficient for: Any environment that permits the desired activity, for example, that permit specific binding or hybridization between two nucleic acid molecules (such as an NPPF and a modified nucleic acid, or an NPPF and a CFS), permit specific binding between an antibody and a modified nucleotide, or that permit a nuclease to remove (or digest) unbound nucleic acids.

Contact: Placement in direct physical association; includes both in solid and liquid form. For example, contacting can occur in vitro with a nucleic acid probe (e.g., an NPPF) or an antibody, and biological sample (e.g., a cell lysate) in solution.

Detect: To determine if an agent (such as a signal, particular nucleotide, amino acid, nucleic acid molecule, nucleic acid modification, and/or organism) is present or absent. In some examples, this can further include quantification. For example, use of the disclosed methods permit detection of modified nucleic acid molecules in a sample.

Detectable label: A compound or composition that is conjugated directly or indirectly to another molecule (such as a nucleic acid molecule, for example an NPPF or an amplification primer/probe) to facilitate detection of that molecule. Specific, non-limiting examples of labels include fluorescent and fluorogenic moieties, chromogenic moieties, haptens, affinity tags, and radioactive isotopes. In one example the label is biotin. The label can be directly detectable (e.g., optically detectable) or indirectly detectable (for example, via interaction with one or more additional molecules that are in turn detectable). Exemplary labels in the context of the probes disclosed herein are described below. Methods for labeling nucleic acids, and guidance in the choice of labels useful for various purposes, are discussed, e.g., in Sambrook and Russell, in Molecular Cloning: A Laboratory Manual, 3^{rd} Ed., Cold Spring Harbor Laboratory Press (2001) and Ausubel et al., in Current Protocols in Molecular Biology, Greene Publishing Associates and Wiley-Intersciences (1987, and including updates).

Enriched: A solution or composition which has been processed to have a higher purity and concentration of a target compound as compared to the nonprocessed solution or composition it was derived from, where the nonprocessed composition contains both target and nontarget compounds (e.g., modified and non-modified nucleic acid molecules, respectively).

Hybridization: The ability of complementary single-stranded DNA, RNA, or DNA/RNA hybrids to form a duplex molecule (also referred to as a hybridization complex). Nucleic acid hybridization techniques can be used to form hybridization complexes between a nucleic acid probe, and the gene it is designed to target.

“Specifically hybridizable” and “specifically complementary” are terms that indicate a sufficient degree of complementarity such that stable and specific binding occurs between a first nucleic acid molecule (or its analog) and a second nucleic acid molecule (such as a nucleic acid target, for example, a DNA or RNA target). The first and second nucleic acid molecules need not be 100% complementary to be specifically hybridizable. Specific hybridization is also referred to herein as “specific binding.”

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, NY (chapters 9 and 11).

Isolated: An “isolated” biological component (such as modified nucleic acid molecules) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated modified nucleic acid molecules, in some examples are at least 50% pure, such as at least 75%, at least 80%, at least 90%, at least 95%, at least 98%, or 100% pure, for example relative to the modified nucleic acid molecules purity prior to their isolation.

Nuclease: An enzyme that cleaves a phosphodiester bond. An endonuclease is an enzyme that cleaves an internal phosphodiester bond in a nucleotide chain (in contrast to exonucleases, which cleave a phosphodiester bond at the end of a nucleotide chain). Endonucleases include restriction endonucleases or other site-specific endonucleases (which cleave DNA at sequence specific sites), DNase I, Bal 31 nuclease, S1 nuclease, Mung bean nuclease, Ribonuclease A, Ribonuclease T1, RNase I, RNase PhyM, RNase U2, RNase CLB, micrococcal nuclease, and apurinic/apyrimidinic endonucleases. Exonucleases include exonuclease III and exonuclease VII. In particular examples, a nuclease is specific for single-stranded nucleic acids, such as S1 nuclease, Mung bean nuclease, Ribonuclease A, or Ribonuclease T1.

Nucleic acid: A deoxyribonucleotide or ribonucleotide polymer in either single or double stranded form, and unless otherwise limited, encompassing analogs of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in a polynucleotide. A nucleotide sequence refers to the sequence of bases in a polynucleotide.

A modified nucleic acid (such as a modified DNA or RNA) is a nucleic acid molecule having one or more nucleotides or ribonucleotides that have been altered via a chemical modification or isomerization, whose detection, amount, or sequence is to be determined (for example in a quantitative or qualitative manner). These modifications may serve as an additional layer(s) of regulation for the modified nucleic acid. Three classes of proteins interact with modifications: writer proteins add the modification, eraser proteins remove it, and reader proteins recognize the modification or a nucleic acid with a modification. Modification of a nucleic acid, therefore, is a dynamic event which is regulated in response to various cellular events, such as stress, cell cycle stage, disease, or treatment with a drug or other agent. In one example, modification may be a response to treatment with an agent (such as a pharmaceutical or biological agent). Exemplary modifications include methylation of a nucleotide or ribonucleotide, such as N⁶-methyladenosine, 1-methylguanosine, 4-methylcytidine, 5-hydroxymethylcytosine, 5-methylcytosine, 5-methyluridine, 7-methylguanosine, N¹-methyladenosine, N⁴-acetylcytidine, N⁶,2′-O-dimethyladenosine, N⁶,N⁶-dimethyladenosine, or adenine-to-inosine editing. Thus in some examples, a modified DNA or RNA includes one or more N⁶-methyladenosines. In one example, a defined region or particular portion of a nucleic acid molecule is modified. In an example where the modified nucleic acid molecule is a modified DNA or a modified RNA, such a modified nucleic acid molecule can be defined by its specific sequence or function; by its gene or protein name; or by any other means that uniquely identifies it from among other nucleic acids.

In some examples, modification of a nucleic acid molecule (e.g., a DNA or RNA) is “associated with” a disease or condition. That is, detection of the modified nucleic acid molecule can be used to infer the status of a sample with respect to the disease or condition. For example, the nucleic acid molecule can exist in two (or more) distinguishable forms, such that a first form correlates with absence of a disease or condition and a second (or different) form correlates with the presence of the disease or condition. The two different forms can be qualitatively distinguishable, such as by presence or absence of a modification, or percent modification of the pool of a specific nucleic acid, and/or the two different forms can be quantitatively distinguishable, such as by the number of copies (or expression level) of the modified nucleic acid sequence that are present in a sample.

Nucleotide: The fundamental unit of nucleic acid molecules. A nucleotide includes a nitrogen-containing base attached to a pentose monosaccharide with one, two, or three phosphate groups attached by ester linkages to the saccharide moiety.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5-triphosphate (UTP or U).

In one example, nucleotides include those nucleotides containing modified bases, modified sugar moieties and modified phosphate backbones, for example as described in U.S. Pat. No. 5,866,336 to Nazarenko et al. (herein incorporated by reference). The term also includes nucleotides containing other modifications, such as found in locked nucleic acids (LNAs). Thus, the NPPFs, primers, and CFSs, disclosed herein can include natural and unnatural bases.

Examples of modified base moieties which can modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, N⁶-methyladenosine, and 2,6-diaminopurine. In one example, the modified base is N⁶-methyladenosine.

Examples of modified sugar moieties which may modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Primer: A short nucleic acid molecule, such as a DNA oligonucleotide 9 nucleotides or more in length, which in some examples is used to initiate the synthesis of a longer nucleic acid sequence. Longer primers can be about 10, 12, 15, 20, 25, 30 or 50 nucleotides or more in length. Primers can be annealed to a complementary nucleic acid strand by nucleic acid hybridization to form a hybrid between the primer and the complement strand, and then the primer extended along the complement strand by a polymerase enzyme. Primer pairs can be used for amplification of a nucleic acid sequence, for example by PCR or other nucleic-acid amplification methods.

In one example, a primer includes a label, which can be referred to as a probe.

Probe: A nucleic acid molecule capable of hybridizing with a target nucleic acid molecule (e.g., DNA or RNA) and, when hybridized to the target, is capable of being detected either directly or indirectly. Thus probes permit the detection, and in some examples quantification, of modified nucleic acid molecules, such as a DNA or RNA containing N⁶-methyladenosine. In some examples, a probe includes a detectable label.

Nuclease protection probe (NPP): A nucleic acid molecule having a sequence that is complementary to a DNA or RNA and is capable of hybridizing to the DNA or RNA. The NPP protects the complementary DNA or RNA nucleic acid molecule from cleavage by a nuclease, such as a nuclease specific for single-stranded nucleic acids. A nuclease protection probe comprising a flanking sequence (NPPF) is an NPP that further includes one or more flanking sequences at the 5′-end, 3′-end, or both, wherein the flanking sequence includes a sequence of contiguous nucleotides not found in a nucleic acid molecule present in the sample, and which can provide a universal amplification sequence point that can be used as an attachment point for an amplification primer. The region of the NPP complementary to a DNA or RNA to be sequenced need not be 100% complementary for example at least 80%, at least 85%, at least 90%, or at least 95% complementarity can be sufficient.

Sample: A biological specimen containing DNA (for example, genomic DNA or cDNA), RNA (including mRNA or miRNA), protein, or combinations thereof, obtained from a subject (such as a human or other mammalian subject). Examples include, but are not limited to cells, chromosomal preparations, peripheral blood or fractions thereof, urine, saliva, tissue biopsy (such as a tumor biopsy or lymph node biopsy), surgical specimen, bone marrow, amniocentesis samples, fine needle aspirates, circulating tumor cells, and autopsy material. Also includes cell lysates generated from any such samples. In one example, a sample includes modified RNA, modified DNA, or both. In particular examples, samples are used directly (e.g., fresh or frozen), or can be manipulated prior to use, for example, by fixation (e.g., using formalin) and/or embedding in wax (such as FFPE tissue samples). In one example, a sample, such as a cell lysate, includes DNA and/or RNA molecules containing one or more N⁶-methyladenosines.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are.

Methods of alignment of sequences for comparison are available. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, Comput. Appl. Biosci. 5:151-3, 1989; Corpet et al., Nucl. Acids Res. 16:10881-90, 1988; Huang et al. Comput. Appl. Biosci. 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mot. Biol. 215:403-10, 1990) is available from several sources, including the National Center for Biological Information (NCBI, National Library of Medicine, Building 38A, Room 8N805, Bethesda, MD 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

One indication that two nucleic acid molecules are closely related is that the two molecules hybridize to each other under stringent conditions. Stringent conditions are sequence-dependent and are different under different environmental parameters. For example, sequences having at least 80%, at least 85%, at least 90%, at least 91%, at least 92%, at least 93%, at least 94%, at least 95%, at least 96%, at least 97%, at least 98%, or at least 99%, such as 100% sequence identity can indicate that the sequences are similar.

Sequencing: To determine the primary structure (or primary sequence) of an unbranched biopolymer. Sequencing results in a symbolic linear depiction known as a sequence which succinctly summarizes much of the atomic-level structure of the sequenced molecule, for example, a polynucleotide. When the molecule is a polynucleotide, such as, for example, RNA or DNA, sequencing can be used to obtain information about the molecule at the nucleotide level, which can then be used in deciphering various secondary information about the molecule itself and/or the polypeptide encoded thereby. DNA sequencing is the process of determining the nucleotide order of a given DNA molecule and RNA sequencing is the process of determining the nucleotide order of a given RNA molecule. In some examples, sequencing of a nucleic acid molecule is done indirectly, for example by determining the sequence of at least a portion of a nuclease protection probe comprising a flanking sequence (NPPF), which hybridized to the nucleic acid molecule. In some examples, the disclosed methods are used to directly or indirectly sequence modified nucleic acid molecules, such as DNA or RNA containing one or more N⁶-methyladenosines.

Simultaneous: Occurring at the same time or substantially the same time and/or occurring in the same sample or the same reaction (for example, contemporaneous). In some examples, the events occur within 1 microsecond to 120 seconds of one another (for example within 0.5 to 120 seconds, 1 to 60 seconds, or 1 to 30 seconds, or 1 to 10 seconds).

Subject: Any multi-cellular vertebrate organism, such as human and non-human mammals (e.g., veterinary subjects). In one example, a subject is known or suspected of having a tumor or an infection. In some examples, a subject is a source of a sample to be analyzed.

Surface (or substrate): Any solid support or material which is insoluble, or can be made insoluble by a subsequent reaction. Numerous and varied solid supports can be used in the methods provided herein and include, without limitation, nitrocellulose, nylon, the walls of wells of a reaction tray, multi-well plates, test tubes, membranes, beads (such as polystyrene beads, magnetic beads, protein A beads, protein G beads, glass beads, etc.), and microparticles (such as latex particles). Any suitable porous material with sufficient porosity to allow access by detector reagents and a suitable surface affinity to immobilize capture reagents (e.g., oligonucleotides, proteins, or antibodies) is contemplated by this term. Microporous structures are useful, as are materials with gel structure in the hydrated state.

Further examples of useful solid supports include natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

### III. Methods of Isolating Modified Nucleic Acid Molecules

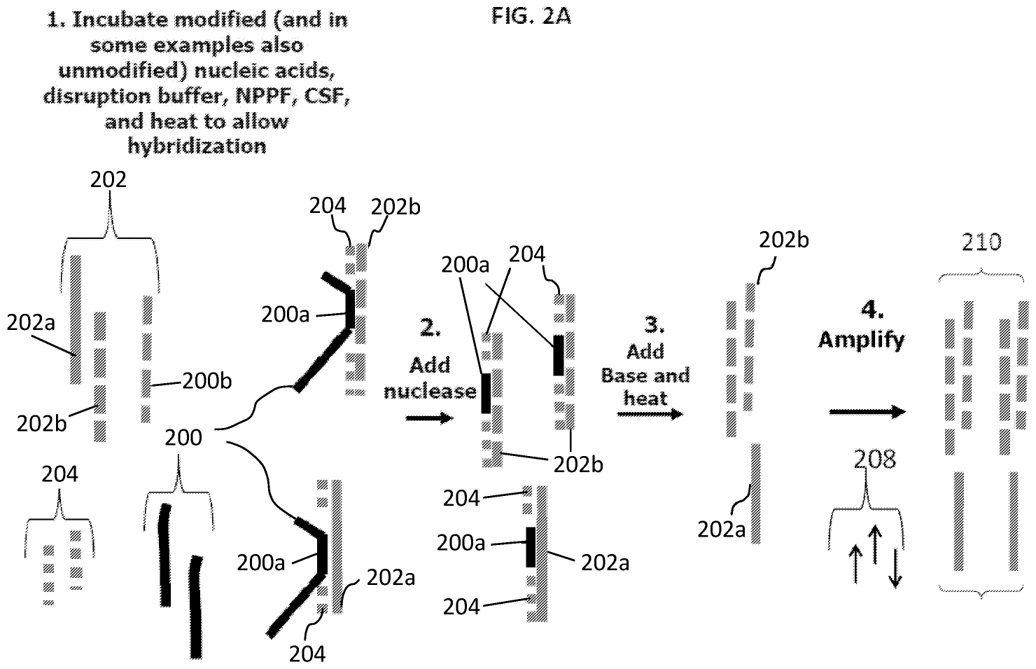

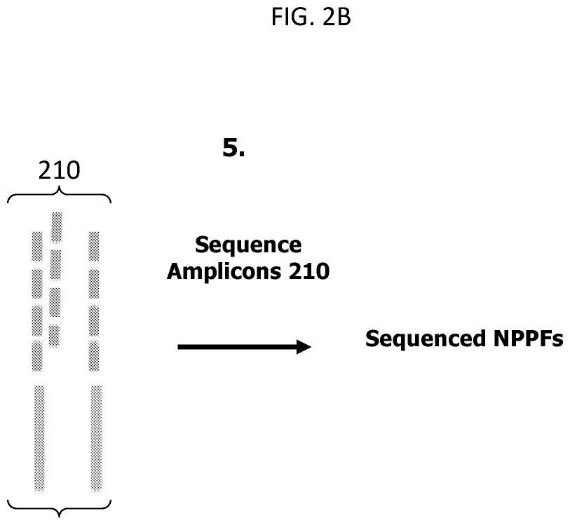

Disclosed herein are methods for isolating or enriching modified nucleic acids from a sample containing both modified and unmodified nucleic acid molecules, which can also be sequenced directly or indirectly (e.g., using NPPFs). In some examples, the modified nucleic acid is RNA containing one or more N⁶-methyladenosine modified bases. The modified nucleic acid is specifically recognized and bound by an antibody optionally attached to a solid support (such as a bead). In some examples, the antibody is an anti-N⁶-methyladenosine antibody, and the optional solid support is a bead. The modified nucleic acid captured and bound to the antibody can optionally be subsequently released from the antibody. In some examples, the antibody is digested (e.g., with proteinase K) or heated in the presence of a protein denaturant, thereby releasing the modified nucleic acids and producing isolated/purified/enriched modified nucleic acids (for example in a sample enriched for modified nucleic acids). The sequence of the isolated modified nucleic acids can then be determined, for example using NPPFs and quantitative nuclease protection sequencing (qNPS) (e.g., see FIGS. 2A-2B).

In some examples, the disclosed methods provide unexpected and superior results as compared to currently available methods for identifying modified nucleic acid molecules. The disclosed methods provide high reproducibility, have fewer steps, allow for easier data analysis, increased specificity, or combinations thereof. For example, McIntyre et al., (Scientific Reports:106590, 2020) discusses the current problems with m⁶A-sequencing, namely that the reproducibility within and between experiments is only about 30-80%. In contrast, the disclosed methods provide increased reproducibility within and between experiments, such as a reproducibility of at least 70%, at least 75%, at least 80%, at least 85%, at least 90%, or at least 95%, such as about 70-95%. In some examples, the disclosed methods provide increased specificity (such as increased signal to noise) as compared to other methods. In some examples, the disclosed methods are at least 10%, at least 20%, at least 30%, at least 40%, at least 50%, at least 75%, at least 90%, at least 95%, at least 100%, at least 200%, at least 300%, at least 400%, or at least 500% more specific than other methods.

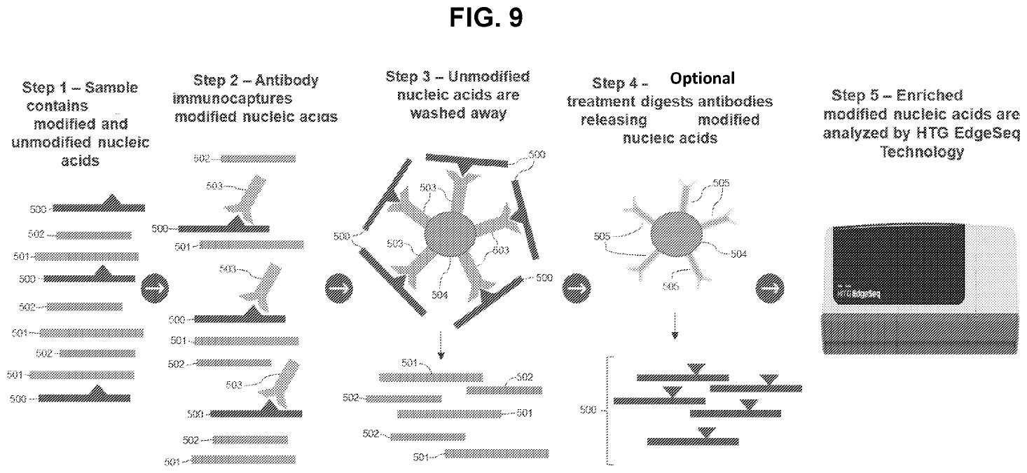

An exemplary embodiment for isolating and determining the sequence of modified nucleic acids is shown in FIG. 9. As shown in FIG. 9, step 1 shows a complex mixture (such as a cell lysate sample, which in some examples has been treated with DNAase), which includes both modified nucleic acids 500 (such as N⁶-methyladenosine modified RNA molecules) and unmodified nucleic acids 501, 502. As shown in step 2, the sample is contacted with a specific binding agent that specifically binds to the modified nucleotide in modified nucleic acids 500 (such as antibody 503, for example an anti-N⁶-methyladenosine antibody), which is optionally attached to a solid support 504 (such as a bead, illustrated in step 3). Antibody 503 permits immunocapture of modified nucleic acids 500 in the complex mixture, as antibody 503 selectively binds to the modified nucleotide present in the modified nucleic acids 500 (but does not bind to unmodified nucleic acids 501, 502 as those do not include the modified nucleotide). As shown in step 3, the unmodified nucleic acids 501, 502 are removed or separated away from the modified nucleic acids 500 (such as by washing away, for example by centrifugation and removal of the supernatant). At this stage, the now enriched or purified modified nucleic acids 500 can be separated from antibody 503 and the optional solid support 504 (e.g., see step 4), or alternatively the modified nucleic acid:antibody complexes or modified nucleic acid:antibody:solid support complexes can be used directly without a separate separation step. In one example, as shown in optional step 4, antibody 503 from step 3 is separated from modified nucleic acids 500. For example, digestion (e.g., with proteinase K), denaturing, and/or heating (e.g., 90-100° C. for 5-30 minutes, such as 95° C. for 10-20 minutes) can produce degraded antibodies 505, which release the previously bound modified nucleic acids 500. As shown in step 5, the released modified nucleic acids 500 can be collected (such as by removing the antibodies and the solid support, for example by centrifugation and collection of the supernatant or by capture of magnetic beads and collection of the supernatant), and the released modified nucleic acids 500 sequenced directly or indirectly (e.g., with HTG EdgeSeq technology, see FIGS. 2-6; available from HTG Molecular Diagnostics, Inc. of Tucson, Arizona). Alternatively, the modified nucleic acid:antibody complexes or modified nucleic acid:antibody:solid support complexes generated in step 3 are used directly and incubated with NPPFs and CFSs and heated in the presence of a lysis buffer (such as one containing SDS and/or formamide), and the released modified nucleic acids 500 sequenced directly or indirectly (e.g., with HTG EdgeSeq technology, e.g., FIGS. 2-4). In on example, next generation sequencing is used, such as HTG's EdgeSeq system. In the HTG EdgeSeq system for sequencing, sample barcodes and sequencing adapters are added to the NPPFs using PCR, the PCR reaction cleaned up to remove excess primers, the tagged amplicons are quantitated and pooled to generate a sequencing library, the tagged amplicons are sequenced using next generation sequencing, and the data assembled (e.g., using software available from HTG Molecular Diagnostics, Inc. of Tucson, Arizona).

### A. Exemplary Modifications

Exemplary modifications that can be present in a nucleic acid molecule whose sequence is determined with the disclosed methods include, but are not limited to: N⁶-methyladenosine, (2′-O)-methylation, pseudouridine, adenosine-to-inosine modification, 1-methyladenosine, N⁶, 2′-O-dimethyladenosine, N²,N²-dimethylgyanosine, 7-methylguanosine, queuosine, peroxywybutosine, N⁴-acetylcytidine, 5-hydroxymethylcytidine, 3-methylcytidine, 5-methylcytidine, dihydrouridine, 5-methoxycarbonylmethyl-2-thiouridine, 5-carbamoylmethyluridine, 2-thiouridine, 1-methylguanosine, 4-methylcytidine, 5-methyluridine, N¹-methyladenosine, inosine, and/or N⁶,N⁶-dimethyladenosine. In one example, the modified nucleic acid includes one or more N⁶-methyladenosine bases.

Other exemplary nucleic acid modifications that can be present in a modified nucleic acid, and thus sequenced by the disclosed methods include 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and/or 2,6-diaminopurine.

Other exemplary nucleic acid modifications that can be present in a modified nucleic acid, and thus sequenced by the disclosed methods include modified sugar moieties, such as arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, and/or a formacetal or analog thereof.

### B. Exemplary Antibodies and Other Specific Binding Agents

The specific binding agent is specific for the modified nucleotide or base present in the modified nucleic acid molecule to be sequenced. Thus, a specific binding agent is one that can specifically bind to any of the exemplary modifications listed above.

In one example, the specific binding agent used to capture a modified nucleic acid molecule is an antibody or antibody fragment. In one example the antibody is a polyclonal antibody. In another example, the antibody is a monoclonal antibody. In one example, the specific binding agent used to capture a modified nucleic acid molecule is an aptamer or aptazyme.

In one example, the antibody used is specific for N⁶-methyladenosine (m⁶A), such as E1610S from New England Biolabs, ab151230 or ab208577 from Abcam, MA5-33030 from ThermoFisher Scientific, MABE1006 or SAB5600251 from Millipore Sigma, 56593S from Cell Signaling Technology, or A01801-202 from EpiGentek.

### C. Exemplary Reaction Conditions

Exemplary reaction conditions that can be used to capture modified nucleic acid molecules present in a sample containing a mixture of modified nucleic acid molecules and unmodified nucleic acid molecules (e.g., steps 1-4 of FIG. 9) include the following.

In some examples, the sample analyzed (e.g., cell lysate), includes (or was generated from) at least 100 cells, at least 250 cells, at least 500 cells, at least 750 cells, at least 1,000 cells, at least 5000 cells, at least 10,000 cells, at least 20,000 cells, at least 50,000 cells, at least 80,000 cells, at least 100,000 cells, at least 120,000 cells, at least 140,000 cells, at least 200,000 cells, or at least 500,000 cells, such as about 100 to 500,000 cells, about 1000 to 500,000 cells, about 100 to 500 cells, about 100 to 1000 cells, about 1000 to 10000 cells, about 10,000 to 80,000 cells, about 20,000 to 150,000 cells, or about 50,000 to 150,000 cells. In some examples, the sample is treated prior to incubation with the specific binding agent, such as diluted, concentrated, washed, treated with DNAse, treated with Proteinase K (or heated in the presence of a protein denaturant), treated with RNasin, or combinations thereof. In one example the sample is treated with a lysis buffer, such as one containing SDS and/or formamide, and heated (e.g., to 90-100° C. for at least 5 minutes, such as to about 95° C. for 5-30 minutes, such as 95° C. for 10-15 minutes), to generate a cell lysate. In one example, the sample is treated with DNAase. In one example the sample is treated with RNasin, such as 1 μL RNasin to prevent RNase activity. In one example, the sample is treated with DNAase and RNasin.

In some examples, the sample includes at least 0.01 fmol of modified nucleic acid, such as at least 0.05 fmol, at least 0.1 fmol, at least 0.5 fmol, at least 1 fmol, at least 5 fmol, at least 10 fmol, at least 50 fmol, at least 100 fmol, at least 500 fmol, or at least 1000 fmol of modified nucleic acid.

The immunocapture step (e.g., step 2, FIG. 9) can include incubation of the sample (such as a cell lysate) with an antibody specific for the modification present in the modified nucleic acids (e.g., anti-m⁶A) for at least 30 minutes, at least 60 minutes, or at least 120 minutes. In some examples, the incubation is performed at room temperature, such as about 20-30° C., such as about 22-28° C. In some examples, the antibody is conjugated to a solid support, such as protein A beads, protein G beads, magnetic beads, or latex beads. Thus, in some examples, conjugated anti-m⁶A protein G beads are used to capture modified nucleic acids containing m⁶A bases. In some examples, the incubation at this step includes mixing, such as end-over-end mixing.

The washing/separation step (e.g., step 3, FIG. 9) to separate modified nucleic acids bound to the antibody from unbound unmodified nucleic acids can include washing the reaction mixture with a wash buffer and removing the unbound unmodified nucleic acids. For example, if the solid support is a multi-well plate, the plate can be washed and the unbound unmodified nucleic acids removed (while the modified nucleic acids bound to the antibody remain attached to the plate). In one example, if the solid support is a bead, the sample can be washed and centrifuged to precipitate the solid support containing the modified nucleic acids bound to the antibody, and the resulting supernatant containing the unbound unmodified nucleic acids removed and the pellet containing the bound modified nucleic acids retained. In one example, if the solid support is a magnetic bead, the sample can be washed and the beads captured using a magnet to separate the solid support containing the modified nucleic acids bound to the antibody, and the resulting supernatant containing the unbound unmodified nucleic acids removed and the captured bead pellet containing the bound modified nucleic acids retained. In one example, the wash buffer includes 150 mM NaCl, 0.1% IGEPAL CA-630, and 10 mM Tris pH 7.4, 1 mM EDTA. In one example, the wash buffer includes 50 mM NaCl, 0.1% IGEPAL CA-630, and 10 mM Tris pH 7.4. In one example, the wash buffer includes 500 mM NaCl, 0.1% IGEPAL CA-630, and 10 mM Tris pH 7.4. In some examples, different wash buffers are used sequentially (e.g., 3 washes with 3 different buffers)).

In some examples the solid support containing the modified nucleic acids bound to the antibody, or the modified nucleic acid:antibody complexes, following separation away from the unmodified nucleic acids, is incubated with NPPFs and CSFs in the presence of a lysis buffer (e.g., such as one containing SDS and/or formamide) and heated (e.g., to 90-100° C. for at least 5 minutes (such as to about 95° C. for 5-30 minutes, such as 95° C. for 10-15 minutes) to separate the modified nucleic acids from the antibody (and the solid support if there is one). The reaction is then incubated to permit hybridization between the NPPFs and CSFs and the NPPFs to its corresponding modified nucleic acid as described herein. Thus, in some examples, steps 1-3 illustrated in FIG. 9 are performed, and following removal of the the unmodified nucleic acids, the reaction proceeds directly to step 1 in FIG. 2A (e.g., denaturation and hybridization).

In another example, as illustrated in step 4, FIG. 9, the solid support containing the modified nucleic acids bound to the antibody, or the modified nucleic acid:antibody complexes, following separation away from the unmodified nucleic acids, is treated to separate the modified nucleic acids from the antibody (and the solid support if there is one). In one example, this step includes incubating the mixture with a reagent that will digest to remove the antibody (or other specific binding agent), without digesting the modified nucleic acids. In one example, proteinase K is used to digest the antibodies, thereby releasing the modified nucleic acids. In one example, this step includes heating the reaction in the presence of a protein denaturant, thereby releasing the modified nucleic acids. For example, the modified nucleic acids bound to the antibody, or the modified nucleic acid:antibody complexes, are incubated in a lysis buffer and heated (e.g., to 90-100° C. for at least 5 minutes (such as to about 95° C. for 5-30 minutes, such as 95° C. for 10-15 minutes). This releases the modified nucleic acids from the antibody, and the resulting modified nucleic acids can optionally be separated away from the antibody (and the solid support if there is one). For example, if the solid support is a bead, the sample can be washed and centrifuged to precipitate the solid support and the resulting supernatant containing the modified nucleic acids retained and subsequently sequenced, for example using the methods provided herein (e.g., see FIGS. 2-4). In some examples, the solid support is a magnetic bead, and the beads are captured using magnets, such as a magnetic column, and the remaining eluate or supernatant containing the modified nucleic acids retained and subsequently sequenced, for example using the methods provided herein (e.g., see FIGS. 2-4). The resulting population of captured modified nucleic acids in some examples is further purified, for example using a commercial DNA or RNA purification kit, such as guanidinium/phenol methods. The population of captured modified nucleic acids can then be incubated with NPPFs and CSFs in the presence of a lysis buffer and heated (e.g., 90-100° C. for at least 5 minutes (such as to about 95° C. for 5-30 minutes, such as 95° C. for 10-15 minutes) (see step 1 of FIG. 2A).

### D. Exemplary Methods of Testing Test Agents and Conditions

The disclosed methods of analyzing modified nucleic acids that include one or more modifications, such as one or more bases modified with m6A, can be used to determine if an agent has targeted effects, off target effects, or both. For example, agents developed for therapeutic uses (e.g., pharmaceutical agents) can be developed for their ability to have a specific modifying or un-modifying effect on a particular nucleic acid (e.g., gene sequence). However, some such agents not only affect the desired sequence (e.g., DNA or RNA target), but have undesired and/or unrecognized modifying/unmodifying effects on one or more other nucleic acid molecules. Agents tested may directly or indirectly modify/unmodify a nucleic acid molecule (for example they may act on DNA, RNA, or protein, but ultimately modify particular nucleic acid molecules), or may inhibit interaction of a modified DNA or RNA with another DNA, RNA, or protein. Thus, agents may result in dysregulation by various mechanisms. In some examples the test agent adds a nucleic acid modification. In some examples the test agent removes a nucleic acid modification. In some examples, the test agent inhibits the regulation of a modified RNA (e.g., an agent that targets a reader protein may inhibit the ability of the reader to either recognize the modified RNA, or may cause the reader to have an opposite-from-normal effect on the RNA (for instance instead of degrading the RNA it may stabilize the RNA, or instead of transporting the RNA somewhere, it may not)). Thus, agents tested include any treatment or condition that results in a change in modification of a nucleic acid molecule. In some examples the test agent or condition is stress (such as an environmental stress) or a disease. The disclosed methods allow for the identification of undesired and/or previously unrecognized modifying/unmodifing effects.

Exemplary test agents that can be used in the disclosed methods include one or more mutagens, microbes, and therapeutic agents. Exemplary mutagens include radiation (e.g., UV radiation), x rays, metals (such as arsenic and cadmium), and chemicals (such as ethidium bromide or other intercalating agents, alkaloids, bromines, sodium azide, alkylating agents, deaminating agent such as nitrous acids, and reactive oxygen species). Exemplary microbes include those that cause disease, such as viruses, bacteria, fungi, and parasites. Exemplary therapeutic agents include pharmaceutical agents that can be used to prevent or treat a disease or a symptom thereof, such as antioxidants, anti-cancer agents, anti-inflammatory agents, anti-viral agents, antibacterial agents, anti-fungal agents, anti-parasitic agents, vaccines, and the like. In some examples, the test agent is one that modifies RNA, such as (1) an agent that directly binds to an RNA, for example exposing or blocking a site that could be modified, or an agent that binds an RNA and dysregulates a pathway, resulting in removal or addition of a modification on a different RNA, or (2) an agent that affects the function or regulation of a reader/writer/eraser protein, which in turn will affect the RNA. In one example the test agent is a stress condition, such as heat shock, cold shock, a disease (such as cancer, diabetes, neurological disease (such as Alzheimer's), autoimmune disease, etc.), wherein modified nucleic acid molecules that result from such a stress condition can be identified using the disclosed methods. Such methods permit identification of on target and off target modifications in the genome of the cell or organism subjected to the stress or test agent.

The methods that examine the effect of a test agent on its ability to modify or unmodify a nucleic acid molecule directly or indirectly can be performed in vitro (e.g., in cell culture), or in vivo (e.g., by administration of the agent to a subject).

In one example, a sample (such as one containing cells) is contacted/incubated/exposed to one or more test agents, such as 1, 2, 3, 4, 5, 6, 7, 8, 9 or 10 different test agents. In some examples, multiple groups of cells are each contacted/incubated/exposed with a different test agent, and the assays performed simultaneously, or contemporaneously (e.g., in multi-well plates, such as 6-, 24, 96- or 384-well plates). In some examples, the sample is an in vitro or ex vivo cell culture (such as a cell culture generated from cells obtained from a subject), such as a culture of normal or diseased cells, such as cancer cells, immune cells, or cells infected with a pathogen (such as bacteria, virus, protozoa, or fungi). The sample can be any cell type, such as mammalian cells, such as human cells (such as cells from any part of the body, such as from the skin, blood, saliva, urine, feces, pancreas, lung, liver, colon, kidney, muscle, CNS, thyroid, ovary, cervix, prostate, breast, bone, esophagus, small intestine, stomach, uterus, and the like. In some examples, the sample is a bacterial sample. Such cells can be cultured in appropriate growth media, such as one including serum or other nutrients, at an appropriate temperature. Following incubation with one or more test agents, modified nucleic acid molecules are captured and sequenced using the methods provided herein.

In one example, a subject is exposed to one or more test agents, such as 1, 2, 3, 4, 5, 6, 7, 8, 9 or 10 different test agents. Exemplary subjects include mammals, reptiles, fish, and birds, such as human and veterinary subjects (including non-human primates, rats, mice and rabbits). For example, the subject can be administered (e.g., iv, im, ip, sc, intratumoral, etc.) one or more test agents or subjected to exposure to one or more test agents. In some examples, the subject was previously exposed to the test agent. In some examples, the subject has a disease or is at risk for developing a disease in the future. In some examples, the subject has cancer. In some examples, the subject has an infectious disease. In some examples, the subject has an autoimmune disease. In some examples, the subject has a neurological disease. In some examples, following exposure of the subject to the test agent (e.g., administration of the test agent), a sample can be obtained from the subject which includes nucleic acid molecules, such as cells from any part of the body, such as from the skin, blood, saliva, urine, feces, pancreas, lung, liver, colon, kidney, muscle, CNS, thyroid, ovary, cervix, prostate, breast, bone, esophagus, small intestine, stomach, uterus, and the like. In one example, the sample is a biopsy, such as a cancer biopsy. In one example, the sample is a blood sample. From this sample modified nucleic acid molecules are captured and sequenced using the methods provided herein.

In some examples, a parallel sample of the same type of cells is not contacted/incubated/exposed with one or more test agents (or a parallel subject is not treated/administered with the test agent) and can serve as a negative control. For example, such a sample/subject may be treated with a vehicle (e.g., PBS, saline, water, DMSO, or the like) instead of the one or more test agents. In some examples, the negative control is the sample/subject prior to treatment with the test agent. In some examples, a parallel sample/subject is treated with an agent known to affect (e.g., directly or indirectly generate) one or more nucleic acid modifications (or modification removals) and serves as a positive control. In one example, the control includes a cell system (e.g., cell lysate) with the nucleic acid modification present, absent, or both.

In some examples, in addition to sequencing captured modified nucleic acid molecules obtained from the sample (either from the cell culture or from the subject), the sample is also analyzed without capturing modified nucleic acid molecules. Instead, nucleic acid molecules in the sample are sequenced without regard to their modification status (e.g., an unbiased portion where both modified and unmodified nucleic acid molecules are sequenced).

In one example, an in vitro sample is contacted/incubated with one or more test agents for at least 1 minute, at least 5 minutes, at least 10 minutes, at least 30 minutes, at least 1 hour, at least 4 hours, at least 6 hours, at least 8 hours, at least 12 hours, at least 24 hours, at least 48 hours, at least 72 hours, at least 96 hours, at least 7 days, at least 10 days, at least 14 days, at least 30 days, at least 60 days, at least 180 days, or more. Over the course of the incubation/contact, culture media can be removed and replaced with fresh media containing the one or more test agents.

In one example, a subject received one or more doses of the one or more test agents, such as at least 1, at least 2, at least 3, at least 4, at least 5, at least 10, at least 15, at least 20, at least 30, at least 60, or at least 90 doses. In some examples, the doses are separated by at least 1 minute, at least 5 minutes, at least 10 minutes, at least 30 minutes, at least 1 hour, at least 4 hours, at least 6 hours, at least 8 hours, at least 12 hours, at least 24 hours, at least 48 hours, at least 72 hours, at least 96 hours, at least 7 days, at least 10 days, at least 14 days, at least 30 days, at least 60 days, at least 180 days, or more. Over the course of the treatment, samples from the subject can be obtained and analyzed using the methods provided herein.

The treated in vitro cell culture or sample obtained from the treated subject (and appropriate controls) can be lysed and one portion used to capture and sequence modified nucleic acid molecules using the methods provided herein, and another portion used to sequence both modified and unmodified nucleic acid molecules using NPPFs and CFSs as provided herein (e.g., total nucleic acid molecules, an “unbiased sample”). The modified nucleic acid molecules are then directly or indirectly sequenced, allowing for a determination if the one or more test agents has on target modifications/unmodifications, off target modifications/unmodifications, or both. Comparison of the captured and unbiased portions from the sample allows, for each nucleic acid molecule in the sample, a determination of the expression level, a determination of whether a modification is present/absent, and/or a modification percentage to be calculated.

### IV. Methods of Detecting or Sequencing Nucleic Acid Molecules

Disclosed herein are methods of sequencing nucleic acid molecules present in a sample, such as modified nucleic acids that include one or more modifications, such as one or more N⁶-methyladenosine bases. Modified nucleic acids can be sequenced directly, or indirectly using NPPFs and CFSs as provided herein. In some examples, at least two different nucleic acid molecules (e.g., two or more modified nucleic acids) are detected in the same sample or same assay (for example, in the same well of an assay plate or array). In some examples, at least 1000, at least 10,000, at least 20,000, or at least 50,000 different nucleic acid molecules (such as different modified nucleic acid molecules) are detected in the same sample or same assay (for example, in the same well of an assay plate or array). In some examples, the same modified nucleic acid molecule(s) is detected in at least two different samples or assays (for example, in samples from different patients)). In addition to sequencing modified nucleic acids that have been isolated from a sample (or a portion of a sample), in some examples the methods also include sequencing both modified nucleic acids and unmodified nucleic acids in the sample (e.g., the modified nucleic acids are not removed or separated from the unmodified nucleic acids), for example to permit comparisons between the two portions of the sample analyzed. Thus, in some examples a sample, such as a cell lysate, is divided into two or more different portions, wherein at least one portion is treated to capture the modified nucleic acid molecules therein (e.g., see FIG. 9), and at least one portion is not treated to capture the modified nucleic acid molecules therein and instead both modified and unmodified nucleic acid molecules therein are sequenced.

The disclosed methods provide improvements to current methods used to detect, such as sequence, modified nucleic acid molecules, such as those including N⁶-methyladenosine bases. For example, the disclosed methods have increased reproducibility, such as reproducibility of at least 70%, such as 70-95%. In some examples, the disclosed methods have increased specificity (such as increased signal to noise) as compared to other methods. In some examples, the disclosed methods are at least 10%, at least 20%, at least 30%, at least 40%, at least 50%, at least 75%, at least 90%, at least 95%, at least 100%, at least 200%, at least 300%, at least 400%, or at least 500% more specific than other methods.

In addition, the disclosed methods of sequencing modified (or unmodified) nucleic acid molecules use a nuclease protection probe comprising a flanking sequence (NPPF). The use of NPPF(s) permits multiplexing, as well as conserving the stoichiometry of the sequenced modified (or unmodified) nucleic acid molecule, because the flanking sequences on the probe permit universal primer binding sites for amplification. As the primer binding sites are universal, the same primers can be used to amplify any NPPF for any modified sequence, thus allowing for multiplexing and conservation of stoichiometry. In one example, amplifying from flanking sequences on both ends of the NPPF provides an unexpected and greater specificity than prior methods of sequencing modified nucleic acid molecules. NPPFs with intact 3′- and 5′-flanking sequences will be amplified exponentially; nuclease-cleaved NPPFs will not be amplified sufficiently to be sequenced or detected. The disclosed methods conserve the original nucleic acid molecule stoichiometry such that the detected or sequenced modified nucleic acid molecules retain the same relative quantities of the modified (or unmodified) nucleic acid molecules as in the test sample, such as a variation of no more than 20%, no more than 15%, no more than 10%, no more than 9%, no more than 8%, no more than 7%, no more than 6%, no more than 5%, no more than 4%, no more than 3%, no more than 2%, no more than 1%, no more than 0.5%, or no more than 0.1%, such as 0.001%-5%, 0.01%-5%, 0.1%-5%, or 0.1%-1%.

The disclosed methods also permit multiplexing experiments, such as multiple reactions within the same assay (such as multiple samples from different patients or cell lysates in the same reaction well), and multiple reactions analyzed within the same run/channel of the sequencer.

Specifically, the disclosed sequencing methods use nucleic acid protection probes (NPPs), which include flanking sequences on one or both ends of the NPPs. These NPPs with 5′-end and/or 3′-end flanking sequences are referred to herein as nucleic acid protection probes with flanking sequences (NPPFs). The presence of the one or both flanking sequences, which serve as universal primer points for hybridization and/or amplification (and can be used for other purposes including capture or tagging of NPPFs), conserve the original nucleic acid stoichiometry in the sample as the flanking sequences are part of the NPPF. In addition, this eliminates the need for ligation to add priming sites, tags, and the like to the NPPFs, which can incorporate artifacts which skew the nucleic acid stoichiometry in the sample, and provide an additional source of variability. Eliminating the need for ligation eliminates both potential artifact skewing stoichiometry and degrading reproducibility.

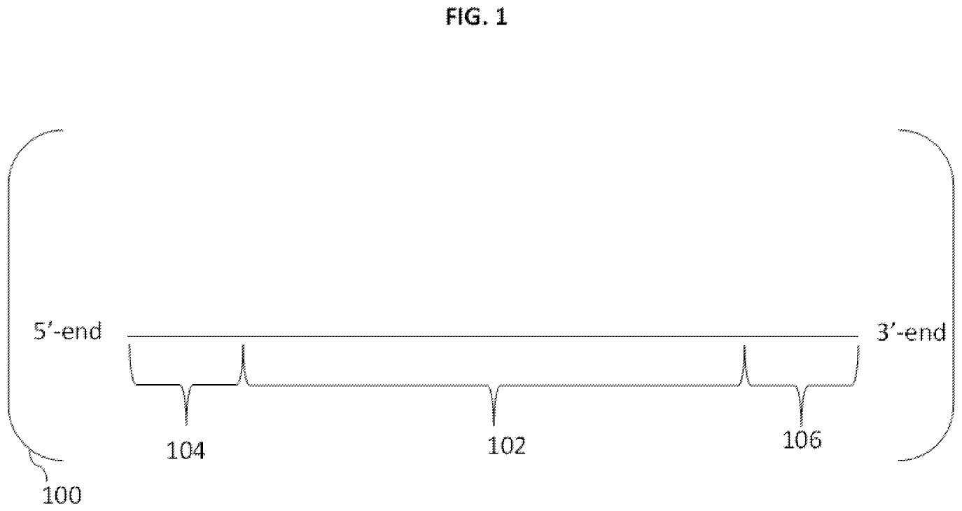

FIG. 1 is a schematic diagram showing an exemplary NPPF. The nuclease protection probe having at least one flanking sequence (NPPF) 100 includes a region 102 that includes a sequence that specifically binds to a particular nucleic acid sequence (such as a modified nucleic acid sequence). The nucleic acid sequence to be sequenced can be DNA (e.g., genomic DNA or cDNA) or RNA (such as mRNA, miRNA, tRNA, siRNA, or ncRNA), or both. The NPPF includes one or more flanking sequences 104 and 106. FIG. 1 shows an NPPF 100 with both a 5′-flanking sequence 104 and a 3′-flanking sequence 106. However, NPPFs can in some examples have only one flanking sequence.

FIG. 2A is a schematic diagram showing the steps of an exemplary method of using NPPFs 202 to sequence nucleic acid molecules 200, such as a modified nucleic acid molecule after it is captured from a sample containing both modified and unmodified nucleic acid molecules (e.g., see FIG. 9, step 5), or modified and unmodified nucleic acid molecules present in a sample (e.g., cell lysate) without prior capture/isolation of the modified nucleic acid molecules. As shown in step 1, modified nucleic acid molecules, unmodified nucleic acid molecules, or both, 200, such as a sample or solution containing isolated modified nucleic acid generated using the methods provided herein (e.g., steps 1-4 of FIG. 9) is contacted or incubated with a plurality of nuclease protection probes having one or more flanking sequences (NPPFs) 202 including at least one NPPF which specifically binds to a first nucleic acid (such as a modified DNA or RNA). The reaction can also include other NPPFs which specifically bind to a second nucleic acid, and so on. For example, the method can use one or more different NPPFs designed to be specific for each unique nucleic acid molecule. Thus, the measurement of 100 genes requires the use of at least 100 different NPPFs, with at least one NPPF specific per gene (such as several different NPPFs/gene). Thus, for example, the method can use at least 2 different NPPFs, at least 3, at least 4, at least 5, at least 10, at least 25, at least 50, at least 75, at least 100, at least 1000, at least 10,000, at least 15,000, at least 20,000, at least 50,000, at least 75,000, or at least 100,000 different NPPFs (such as 2 to 500, 2 to 100, 5 to 10, 2 to 10, 2 to 20, 1000 to 100,000, 10,000 to 50,000, or 20,000 to 75,000 different NPPFs). However, one will appreciate that in some examples, the plurality of NPPFs can include more than one (such as 2, 3, 4, 5, 10, 20, 50 or more) NPPFs specific for a single modified or unmodified nucleic acid molecule. FIG. 2A illustrates two species of NPPF in the form of NPPF 202a and NPPF 202b. NPPF 202a is specific for a first modified (or unmodified) nucleic acid molecule and NPPF 202b is specific for second modified (or unmodified) nucleic acid molecule, where the second modified (or unmodified nucleic acid molecule is the same or different than the first modified (or unmodified) nucleic acid molecule. In some examples, the NPPFs include a detectable label, such as biotin, but one skilled in the art will appreciate that a label can be added at other steps, such as during amplification. The reaction also includes nucleic acid molecules that are complementary to the flanking sequences (CFS), 204, that are specific for the flanking sequences of the NPPF 202. FIG. 2A shows the dotted bars 204 as the CFSs specific for a flanking sequence(s) of the NPP. One skilled in the art will appreciate that the sequence of the CFSs will vary depending on the flanking sequence present. In addition, more than one CFS can be used to ensure a flanking region is protected (e.g., at least two CFSs can be used that bind to different regions of a single flanking sequence). The CFSs can include natural or unnatural bases. Although FIG. 2A shows NPPFs with flanking sequences on both ends of the NPPF; one skilled in the art will appreciate that a single flanking sequence can be used. The sample, NPPFs and CFSs are incubated under conditions sufficient for NPPFs to specifically bind to their respective nucleic acid molecule, and for CFSs to bind to its their complementary sequence on the NPPF flanking sequence. In some examples, the CFSs 204 are added in excess of the NPPFs 202, for example at least 5-fold more CFSs than NPPFs (molar excess), such as at least 6-fold, at least 7-fold, at least 8-fold, at least 9-fold, at least 10-fold, at least 20-fold, at least 40-fold, at least 50-fold, or at least 100-fold more CFSs than the NPPFs. In some examples, the NPPFs 202 are added in excess of the total nucleic acid molecules in the sample, for example at least 50-fold more NPPF than total nucleic acid molecules in the sample (molar excess), such as at least 75-fold, at least 100-fold, at least 200-fold, at least 500-fold, or at least 1000-fold more NPPF than the total nucleic acid molecules in the sample. For experimental convenience, a similar concentration of each NPPF can be included to make a cocktail, such that for the most abundant nucleic acid measured there will be at least 50-fold more NPPF for that nucleic acid molecule, such as an at least 100-fold excess. The actual excess and total amount of all NPPFs used is limited only by the capacity of the nuclease (e.g., S1 nuclease) to destroy all NPPF's that are not hybridized to nucleic acid molecules. In some examples the reaction containing NPPFs 202, CFSs 204, and nucleic acid molecules to be sequenced 200 is heated, for example incubated for overnight at about 37-55° C., such as about 40-50° C., such as 40, 41, 42, 43, 44, 45, 46, 47, 48, 49 or 50° C., to allow hybridization to occur.

As shown in step 2 in FIG. 2A, after allowing the binding/hybridization reactions to occur, the reaction is contacted with a nuclease specific for single-stranded (ss) nucleic acid molecules under conditions sufficient to remove (or digest) ss nucleic acid molecules, such as unbound nucleic acid molecules (such as unbound NPPFs, CFSs, and modified (or unmodified) nucleic acid molecules, or portions of such molecules that remain single stranded). As shown in FIG. 2A, incubation of the reaction with a nuclease specific for ss nucleic acid molecules results in degradation of any ss nucleic acid molecules, leaving intact double-stranded nucleic acid molecules, including NPPFs 202a and 202b hybridized to CFSs 204 and portions 200a of nucleic acid molecules 200 whose sequence is to be determined. For example, the reaction can be incubated at 37-55° C. (such as 40-50° C.) for 1.5 hours with S1 nuclease (though hydrolysis can occur at other temperatures and be carried out for other periods of time, and in part that the time and temperature required will be a function of the amount of nuclease, and the amount of nucleic acid required to be hydrolyzed, as well as the Tₘ of the double-stranded region(s) being protected).

After the nuclease reaction, the reaction mixture can optionally be treated to otherwise remove or separate non-hybridized material and/or to inactivate or remove residual enzymes (e.g., by heat, phenol extraction, precipitation, column filtration, etc.). For example, as shown in step 3 the pH of the reaction can be increased to inactivate the nuclease, and the reaction heated to destroy the nuclease. In addition, heating the reaction dissociates the nucleic acid (such as modified DNA or modified RNA) and the CFSs from the complementary regions on the NPPF. This leaves behind the intact NPPFs that previously bound the modified (or unmodified) nucleic acid molecules and CFSs, wherein the intact NPPFs are in direct proportion to how much NPPF had been hybridized to the modified (or unmodified) nucleic acid molecules. In some examples, the hybridized nucleic acid and CFSs can be degraded, e.g., by nucleases or by chemical treatments. Alternatively, the sample can be treated so as to leave the (single strand) hybridized portion 200a of the modified (or unmodified) nucleic acid molecules 200, or the duplex formed by the hybridized modified (or unmodified) nucleic acid molecules and CFSs to the NPPF, to be further analyzed (for example the portion 200a of nucleic acid molecule 200 hybridized to the NPPF can be sequenced). In one example, the pH increased to about pH 8, and the reaction incubated at about 90-100° C. (such as about 95° C.) for about 10 minutes causing the modified (or unmodified) nucleic acid and the CFSs to dissociate (and if the modified (or unmodified) nucleic acid is RNA, hydrolyzing said nucleic acids).

As shown in step 4 in FIG. 2A, either after step 2 or step 3, the NPPFs 202 are amplified, for example using PCR. FIG. 2A shows the PCR primers or probes 208 as arrows. The PCR primers or probes can include a label, such as biotin, thereby resulting in the production of amplicons that are labeled. At least a portion of the PCR primers/probes 208 are specific for the flanking sequences of the NPPFs 202. The resulting amplicons 210 can then be sequenced (see FIG. 2B). In some examples, the concentration of the primers 208 are in excess of the CFSs 204, for example in excess by at least 10,000-fold, at least 50,000-fold, at least 100,000-fold, at least 150,000-fold, at least 200,000-fold, or at least 400,000-fold. In some examples, the concentration of primers 208 in the reaction is at least 200 nM (such as at least 400 nM, at least 500 nM, or at least 1000 nM), and the concentration of CFSs 204 in the reaction is less than 1 pM, is less than 0.5 pM, or is less than 0.1 pM.

As shown in step 5 in FIG. 2B, the amplicons 210, which are the amplified NPPFs, can be sequenced. For example, one or both of the flanking sequences of the amplified NPPFs can include (or have added thereto) a sequence adapter, or a primer that is complementary to and is hybridized to the flanking sequence, can include a sequence adapter sequence, which is complementary to capture sequences for the sequencing platform, and permits sequencing of the NPPF using a particular sequencing chip. In some examples, a plurality of NPPFs are sequenced in parallel, for example simultaneously or contemporaneously. This method can thus be used to sequence a plurality of NPPF sequences, thereby determining the sequences and identity of target modified nucleic acid molecules in the sample.

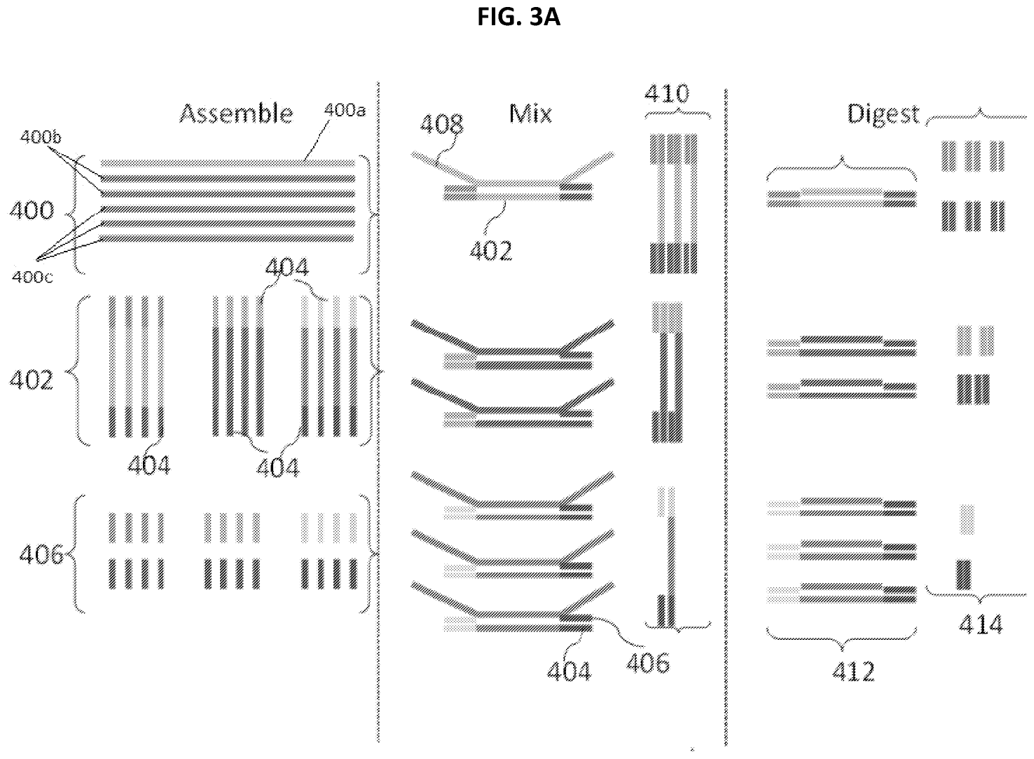

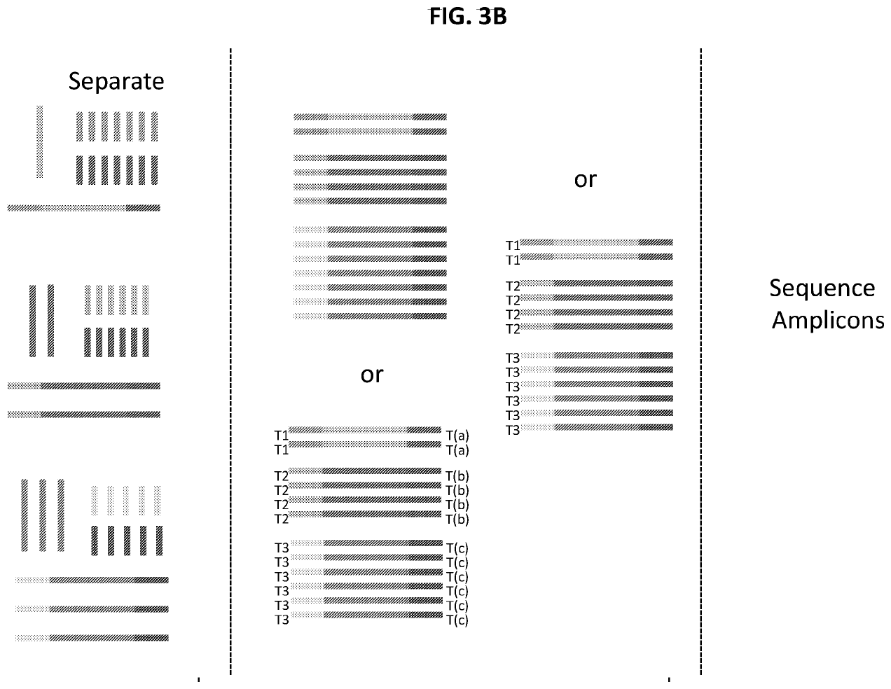

FIGS. 3A and 3B are schematic diagrams providing a further a summary of the sequencing steps, with more details of the nucleic acid molecules. As shown in the left panel of FIG. 3A, nucleic acid molecules 400 (such as modified nucleic acid molecules isolated from a sample, e.g., see steps 1-4 in FIG. 9) is contacted or incubated with a plurality of nuclease protection probes having one or more flanking sequences (NPPFs) 402 (wherein each NPPF is specific for a particular modified (or unmodified) nucleic acid molecule 400), and with nucleic acid molecules that are complementary to the flanking sequences (CFS) 406, that are specific for the flanking sequences 404 on the ends of the NPPFs. Three different nucleic acids 400 are shown: one copy of nucleic acid 1 (top line, 400a) two copies of nucleic acid 2 (2nd and 3^{rd} lines, 400b), and three copies of nucleic acid 3 (4^{th}-6^{th} lines, 400c). This example shows equal amounts of each NPPF 402 are added. Although FIG. 3A shows NPPFs with flanking sequences on both ends of the NPP; one skilled in the art will appreciate that a single flanking sequence can be used. The middle panel of FIG. 3A shows the reaction products after allowing the binding/hybridization reactions to occur between the modified (or unmodified) nucleic acids 400, NPPFs 402, and CFSs 406. The nucleic acids 400 hybridize to a central region of the NPPFs, and the CFSs 406 hybridize to the 3′- and 5′-flanking sequences 404. The right panel of FIG. 3A shows the reaction products after the reaction is contacted with a nuclease specific for single-stranded (ss) nucleic acid molecules under conditions sufficient to remove (or digest) ss nucleic acid molecules. As shown, regions 408 of the modified (or unmodified) nucleic acids that did not hybridize to an NPPF 402 are digested away, as are ss regions of NPPFs that did not bind to a nucleic acid molecule or a CFS (e.g., 410). This leaves intact double-stranded nucleic acid molecules, including NPPFs that have bound thereto and CFSs and modified (or unmodified) nucleic acid molecule (e.g., 412) and well as regions of the NPPF that hybridized to target only (but not CFS), or that hybridized to CFS only (but not target) (e.g., 414).

The left panel of FIG. 3B shows the reaction products after separating the double-stranded nucleic acid molecules (for example using heat and increasing the pH). The resulting NPPFs that survive, which are in direct proportion to the modified (or unmodified) nucleic acid molecules that protected them during the nuclease step, can then be amplified. The middle panel of FIG. 3B shows the reaction products after they are amplified. The right panel of FIG. 3B shows that after amplification, the resulting NPPF amplicons can be detected or sequenced (e.g., see FIGS. 2A-2B).

In some embodiments, the methods can include contacting modified (or unmodified) nucleic acids 200, 400, 500 with plurality of NPPFs including at least one NPPF which specifically binds to a first nucleic acid molecule (such as a first modified RNA) and optionally at least one NPPF which specifically binds to a second nucleic acid molecule (such as a second modified RNA). In some examples, the plurality of NPPFs includes more than one (such as 2, 3, 4, 5, or more) NPPFs specific for a single nucleic acid molecule. In some examples, the plurality of NPPFs can include at least one NPPF (such as at least 1, 2, 3, 4, 5, 10, 15, 20, 25, 50, 75, 100, 200, 300, 500, 1000, 2000, 3000, 5000, 10,000, 15,000, 20,000, 30,000, 40,000, 50,000, 60,000, 100,000 NPPFs or more), wherein each NPPF specifically binds to a single modified (or unmodified) nucleic acid molecule. In another or additional example, the plurality of NPPFs include at least two different NPPF populations (such as 2, 3, 4, 5, 10, 20, or 50 different NPPF sequences), wherein each NPPF population (or sequence) specifically binds to a different modified (or unmodified) nucleic acid molecule.

In some examples, several NPPFs hybridize to different portions of the same modified (or unmodified) nucleic acid, and the number of NPPFs hybridizing to different portions of each nucleic acid can be the same or different. For example, a low expressed modified (or unmodified) nucleic acid may have more NPPFs that hybridize to it relative to a modified (or unmodified) nucleic acid expressed at a higher level, such as four NPPFs hybridizing to a low expressed modified (or unmodified) nucleic acid and a single NPPF hybridizing to a high expressed modified (or unmodified) nucleic acid. In some examples, some of the NPPFs specific for some nucleic acids may not have flanking sequences (e.g., NPPs), and thus may not be amplified, or labeled, or have the appropriate adapters attached, and thus this portion of NPPFs will not be detected or sequenced. Using such a mixture, which can be about 1 to 5, or about 1 to 10, or about 1 to 100, or about 1 to 1,000 NPPFs with flanking sequence to NPPs without flanking sequence, the signal measured, or the number of NPPFs sequenced, can be “attenuated”, such that if there are 10,000 copies of modified (or unmodified) nucleic acid, and a ratio of 1 to 5 is used, then after amplification only ⅕^{th} the number of NPPFs will be sequenced as would have been sequenced had every NPPF contained flanking sequences.

In some examples, the plurality of NPPFs include at least 2, at least 5, at least 10, at least 20, at least 100, at least 1000, at least 10,000, at least 15,000, at least 20,000, at least 40,000, at least 50,000, at least 75,000, or at least 100,000 different NPPFs (such as 2 to 500, 2 to 100, 5 to 10, 2 to 10, 2 to 20, 1000 to 100,000, 10,000 to 50,000, 20,000 to 40,000, 10,000 to 100,000, or 20,000 to 75,000 unique NPPF sequences). The plurality of NPPs can include any combination of NPPFs specific for one or more modified (or unmodified) nucleic acid molecules. The plurality of NPPFs, along with the CFSs, are incubated with the sample or lysate thereof (or nucleic acids obtained from the sample, such as captured modified nucleic acids) under conditions sufficient for the NPPFs to specifically hybridize to their corresponding nucleic acids and their corresponding CFSs. In some examples, the CFSs are added in excess of the NPPFs, such as an at least 2-fold, at least 3-fold, at least 4-fold, at least 5-fold or at least 10-fold molar excess of CFS to NPPF. In some examples, the NPPFs are added in excess of the nucleic acid molecules in the sample, such as an at least 10-fold, at least 50-fold, at least 75-fold, at least 100-fold, at least 250-fold, at least 1,000 fold, at least 10,000 fold, or at least 100,000 fold molar excess or more of NPPF to nucleic acid molecules in the sample. It will be appreciated that if the NPPF for a highly abundant nucleic acid is in excess of 1,000 fold, and the same concentration of each different NPPF is the same, then the excess of NPPF for a low abundant nucleic acid molecule can be many times greater, such as 1,000 times greater for a gene that is 1,000 fold lower abundance than the high abundant nucleic acid molecule.

The hybridized reaction can then be contacted with a nuclease specific for single-stranded nucleic acids (for example, S1 nuclease). The resulting NPPFs that survive, which are in direct proportion to the modified (or unmodified) nucleic acid molecules that protected them during the nuclease step, can then be amplified. For example, amplification primers that include a sequence complementary to the flanking sequence of the NPPF can be used. The resulting NPPF amplicons can then be detected, for example by sequencing. The modified (or unmodified) nucleic acid molecule(s) are identified as present in the sample when their respective NPPF is sequenced.

### A. Exemplary Hybridization Conditions

Disclosed herein are conditions sufficient for a plurality of NPPFs to specifically hybridize to nucleic acid molecule(s) (including modified, unmodified, or both), such as DNAs and RNAs present in a sample from a subject, as well as specifically hybridize to CFS complementary to the flanking sequence(s). For example, the features (such as length, base composition, and degree of complementarity) that will enable a nucleic acid (e.g., an NPPF) to hybridize to another nucleic acid (e.g., a modified DNA or modified RNA or CFS) under conditions of selected stringency, while minimizing non-specific hybridization to other substances or molecules can be determined based on the present disclosure. Characteristics of the NPPFs are discussed in more detail in Section IV, below. Typically, a region of an NPPF will have a nucleic acid sequence (e.g., FIG. 1, 102) that is of sufficient complementarity to its corresponding nucleic acid molecule (which may be modified or unmodified) to enable it to hybridize under selected stringent hybridization conditions, as well as a region (e.g., FIG. 1, 104, 106) that is of sufficient complementarity to its corresponding CFS to enable it to hybridize under selected stringent hybridization conditions. Exemplary hybridization conditions include hybridization at about 37° C. or higher (such as about 37° C., 41° C., 42° C., 50° C., 55° C., 60° C., 65° C., 70° C., 75° C., or higher). Among the hybridization reaction parameters which can be varied are salt concentration, buffer, pH, temperature, time of incubation, amount and type of denaturant such as formamide. For example, nucleic acid (e.g., a plurality of NPPFs) can be added to a solution containing either isolated or enriched modified nucleic acid molecules or a cell lysate containing modified and unmodified nucleic acids which in some examples are not purified or isolated, at a concentration ranging from about 10 pM to about 10 nM (such as about 30 pM to 5 nM, about 100 pM to about 1 nM), in a buffer (such as one containing NaCl, KCl, H₂PO₄, EDTA, 0.05% Triton X-100, or combinations thereof) such as a lysis buffer.

In one example, each NPPF is added to the modified (and/or unmodified) nucleic acid molecules at a final concentration of at least 10 pM, such as at least 20 pM, at least 30 pM, at least 50 pM, at least 100 pM, at least 150 pM, at least 200 pM, at least 500 pM, at least 1 nM, or at least 10 nM. In one example, each NPPF is added to the nucleic acid molecules whose sequence is to be determined at a final concentration of about 30 pM. In another example, each NPPF is added to the nucleic acid molecules whose sequence is to be determined at a final concentration of about 167 pM. In another example, each NPPF is added to the nucleic acid molecules whose sequence is to be determined at a final concentration of about 15 pM. In a further example, each NPPF is added to the nucleic acid molecules whose sequence is to be determined at a final concentration of about 1 nM. In one example, each CFS is added to the nucleic acid molecules whose sequence is to be determined at a final concentration of about at least 6-times the amount of probe, such as at least 10-times or at least 20-times the amount of NPPF (such as 6 to 20 times the amount of NPPF). In one example, each CFS is added at least 1 nM, at least 5 nM, at least 10 nM, at least 50 nM, at least 100 nM, or at least 200 nm, such as 1 to 100, 5 to 100 or 5 to 50 nM. For example, if there are six NPPFs, each at 166 pM, each CFSs can be added at 5 to 50 nM.

If not previously done, the nucleic acids in the reaction are denatured, rendering them single stranded and available for hybridization (for example at about 95° C. to about 105° C. for about 5-15 minutes). By using different denaturation solutions, this denaturation temperature can be modified, so long as the combination of temperature and buffer composition leads to formation of single stranded nucleic acid molecules (e.g., ssDNA). The nucleic acids in or captured from the sample, and the CFSs, are then hybridized to the plurality of NPPFs for about 10 minutes to about 72 hours (for example, at least about 1 hour to 48 hours, about 6 hours to 24 hours, about 12 hours to 18 hours, or overnight) at a temperature of about 4° C. to about 70° C. (for example, about 37° C. to about 65° C., about 42° C. to about 60° C., or about 50° C. to about 60° C.). The hybridization conditions can depend on the particular NPPFs and CFSs used, but are set to ensure hybridization of NPPFs to modified or unmodified nucleic acid molecules and the CFSs. In some examples, the plurality of NPPFs and CFSs are incubated with the modified or unmodified nucleic acid molecules at a temperature of at least about 37° C., at least about 40° C., at least about 45° C., at least about 50° C., at least about 55° C., at least about 60° C., at least about 65° C., or at least about 70° C. In one example, the plurality of NPPFs and CFSs are incubated with the modified or unmodified nucleic acid molecules at about 37° C., at about 41° C., at about 42° C., or at about 50° C.

In some examples, no pre-processing of the sample is required except for cell lysis, and the lysate containing modified and unmodified nucleic acid molecules used directly (e.g., in step 1 of FIG. 2A). In other examples, the sample is lysed and modified nucleic acid molecules captured and separated from nonmodified nucleic acid molecules (e.g., steps 1-4 in FIG. 9) and the purified modified nucleic acid molecules used in step 1 of FIG. 2A.

When the NPPFs are subsequently subjected to PCR (e.g., universal amplification or NPPF-specific amplification such as for real time PCR), the buffers and reagents used for lysis, hybridization of NPPFs to their corresponding nucleic acids, nuclease digestion, and base hydrolysis can be compatible with the polymerase used for amplification.

B. Treatment with Nuclease

Following hybridization of the NPPFs to modified or unmodified nucleic acids and to CFSs, the reaction is subjected to a nuclease protection procedure. NPPFs which have hybridized to a modified or unmodified nucleic acid molecule and (when used) CFS (one or two CFSs, depending if there are both 5′- and 3′-flanking sequence on the NPPF or just one, or no CFS where flanking sequences are not required for amplification or measurement) are not hydrolyzed by the nuclease and can be subsequently amplified, and then detected or sequenced (or both).

Treatment with one or more nucleases will destroy all ss nucleic acid molecules (such as RNA and DNA in the sample that is not hybridized to (thus not protected by) NPPFs, NPPFs that are not hybridized to a corresponding nucleic acid, and (when used) CFSs not hybridized to an NPPF), but will not destroy ds nucleic acid molecules such as NPPFs which have hybridized to CFSs and a modified or unmodified nucleic acid molecule present in the reaction. For example, if the reaction includes a cellular extract or lysate, unwanted nucleic acids, such as genomic DNA, tRNA, rRNA, mRNA, miRNA, and portions of a nucleic acid molecule(s) that are not hybridized to complementary NPPF sequences (such as overhangs), can be substantially destroyed in this step. This leaves behind a stoichiometric amount of modified or unmodified nucleic acid/CFS/NPPF duplex. If the modified or unmodified nucleic acid molecule is cross-linked to tissue that occurs from fixation, the NPPFs hybridize to the cross-linked nucleic acid molecule without the need to reverse cross-linking, or otherwise release the nucleic acid from the tissue to which it is cross-linked.

Conditions can be selected such that single nucleotide differences leading to an unpaired base is not cleaved, or a nuclease can be used which just cleaves unpaired bases up to the ends of the hybridized nuclease protection probe, such as an exonuclease. Conditions can also be selected which will hydrolyze the NPPF sequence at the point of a single unpaired base, and similarly hydrolyze the target modified nucleic acid at that position.

Examples of nucleases include endonucleases, exonuclease, and combinations thereof. Any of a variety of nucleases can be used, including, DNAase, pancreatic RNAse, mung bean nuclease, S1 nuclease, RNAse A, Ribonuclease T1, Exonuclease III, Exonuclease VII, RNAse CLB, RNAse PhyM, RNAse U2, or the like, depending on the nature of the hybridized complexes and of the remainder of nucleic acids and non-target nucleic acid sequences present in the reaction. One of skill in the art can select an appropriate nuclease. In a particular example, the nuclease is specific for single-stranded (ss) nucleic acids, for example S1 nuclease. One advantage of using a nuclease specific for ss nucleic acids, in addition to hydrolyzing excess NPPFs and conferring the stoichiometry of the nucleic acid whose sequence is to be determined to the NPPFs, is to remove such single-stranded (“sticky”) molecules from subsequent reaction steps where they may lead to undesirable background or cross-reactivity. S1 nuclease is commercially available from for example, Promega, Madison, WI (cat. no. M5761); Life Technologies/Invitrogen, Carlsbad, CA (cat. no. 18001-016); Fermentas, Glen Burnie, MD (cat. no. EN0321), and others. Reaction conditions for these enzymes can be optimized empirically.

In some examples, S1 nuclease diluted in a buffer (such as one containing sodium acetate, NaCl, KCl, ZnSO₄, KATHON, or combinations thereof) is added to the hybridized NPPF/nucleic acid molecule mixture and incubated at about 37° C. to about 60° C. (such as about 50° C.) for 10-120 minutes (for example, 10-30 minutes, 30 to 60 minutes, 60-90 minutes, or 120 minutes) to digest non-hybridized nucleic acid molecules from the reaction and non-hybridized NPPFs.

The reaction can optionally be treated to remove non-hybridized material and/or to inactivate or remove residual enzymes (e.g., by heating, phenol extraction, precipitation, column filtration, addition of proteinase K, addition of a nuclease inhibitor, chelating divalent cations required by the nuclease for activity, or combinations thereof). In some examples, the reactions are optionally treated to dissociate the modified or unmodified nucleic acid molecule whose sequence is to be determined and the CFS(s) from its complementary NPPF (e.g., using base hydrolysis and heat). In some examples, after hybridization and nuclease treatment, a modified or unmodified RNA molecule hybridized to the NPPF can be degraded, e.g., by dissociating the duplex with NPPF in base and then destroying the RNA by nucleases or by chemical/physical treatments, such as base hydrolysis at elevated temperature, leaving the NPPF in direct proportion to how much had been hybridized to nucleic acid molecule whose sequence is to be determined. Alternatively, the reaction can be treated so as to leave the (single strand) hybridized portion of the modified or unmodified nucleic acid molecule, or the duplex formed by the hybridized modified or unmodified nucleic acid molecule and the probe, to be further analyzed.

In some examples following incubation with a nuclease, base (such as NaOH or KOH) is added to increase the pH to about 9 to 12 and the sample heated (for example to 95° C. for 10 minutes). This dissociates the nucleic acid molecule whose sequence is to be determined/CFS/NPPFs complexes, leaving the NPPF in a single stranded state, and in the case of RNA, hydrolyzes the RNA molecule whose sequence is to be determined. This step can also neutralize or deactivate the nuclease, such as by raising the pH above about 6.

In some examples the reaction is treated to adjust the pH to about 7 to about 8, for example by addition of acid (such as HCl). In some examples the pH is raised to about 7 to about 8 in Tris buffer. Raising the pH can prevent the depurination of DNA and also prevents many ss-specific nucleases (e.g., S1 nuclease) from functioning fully.

In some examples, the reaction is purified or separated to remove undesired nucleic acid or other molecules, prior to amplification, for example by gel purification or other separation method.

### C. Amplification

The resulting NPPF molecules (or resulting modified or unmodified nucleic acid molecules that have been separated from the NPPF), which are in direct proportion to how much nucleic acid molecule whose sequence is to be determined were present in the sample tested, can be amplified, for example using routine methods such as PCR or other forms of enzymatic amplification or ligation based methods of amplification.

Examples of in vitro amplification methods that can be used include, but are not limited to, quantitative real-time PCR; strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134). In one example, a ligation-based method of amplification is used, wherein the primers are NPPF specific and butt-up together so that they can be ligated together, melted off, and then fresh primers ligated together for a series of cycles. Ligation can be enzymatic or non-enzymatic. If the NPPF flanking sequences are used for hybridization of the primers, the amplification can be universal.

Quantitative real-time PCR is another in vitro method to amplify nucleic acid molecules, such as Applied Biosystems (TaqMan PCR). The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Frster resonance energy transfer (FRET) through space. For real time PCR, the sample of NPPFs can be divided into separate wells or reaction locations, and a different NPPF-specific set of primers is added to each well or reaction location. Using probes (each having a different label) permits multiplexing of real time PCR to measure multiple different NPPFs within a single well, or reaction location.

During amplification of the NPPF, an experiment tag, and/or sequencing adapter can be incorporated as, for instance, part of the primer and extension constructs, for example at the 3′- or 5′-end or at both ends. For example, an amplification primer, which includes a first portion that is complementary to all or part of an NPPF flanking sequence, can include a second portion that is complementary to a desired experiment tag and/or sequencing adapter. Different combinations of experiment tags and/or sequencing adapters can be added to either end of the NPPF. In one example, the NPPF is amplified using a first amplification primer that includes a first portion complementary to all or a portion of the 3′-NPPF flanking sequence and a second portion complementary to (or comprising) a desired sequencing adapter, and the second amplification primer includes a first portion complementary to all or a portion of the 5′-NPPF flanking sequence and a second portion complementary to (or comprising) a desired experiment tag. In another example, the NPPF is amplified using a first amplification primer that includes all or a portion of a first portion complementary to the 3′-NPPF flanking sequence and a second portion complementary to (or comprising) a desired sequencing adapter and a desired experiment tag, and the second amplification primer includes a first portion complementary to all or a portion of the 5′-NPPF flanking sequence and a second portion complementary to (or comprising) a desired experiment tag.

NPPF-specific primers can be used to add sequencing adapters, experiment tags (including tags that permit capture of an NPPF by a substrate), and NPPF tags. The sample of NPPFs can be separated into separate wells or locations containing one or more different NPPF-specific primers, amplified, and then either sequenced separately or combined for sequencing (or detected).

Amplification can also be used to introduce a detectable label into the generated NPPF amplicons (for example if the NPPF was originally unlabeled or if additional labeling is desired), or other molecule that permits detection or quenching. For example, the amplification primer can include a detectable label, hapten, or quencher which is incorporated into the NPPF during amplification. Such a label, hapten, or quencher can be introduced at either end of the NPPF amplicon (or both ends), or anywhere in between.

In some examples, the resulting NPPF amplicons are cleaned up before detection or sequencing. For example, the amplification reaction mixture can be cleaned up before detection or sequencing (e.g., using gel purification, biotin/avidin capture and release, capillary electrophoresis). In one example, the NPPF amplicons are biotinylated (or include another hapten) and captured onto an avidin or anti-hapten coated bead or surface, washed, and then released for detection or sequencing. Likewise, the NPPF amplicons can be captured onto a complimentary oligonucleotide (such as one bound to a surface), washed and then released for detection or sequencing. The capture of amplicons need not be particularly specific, as the disclosed methods eliminate most of the genome or transcriptome, leaving behind the NPPF that had been hybridized to target modified nucleic acid molecule. Other methods can be used to clean up the amplified product, if desired.

The amplified products can also be cleaned up after the last step of amplification, while still double stranded, by a method which uses a nuclease that hydrolyzes single stranded oligonucleotides (such as Exonuclease I), which nuclease can in turn be inactivated before continuing to the next step such as hybridization to a surface.

### D. Sequencing of Amplicons

The resulting NPPF amplicons can be sequenced (e.g., step 5 in FIG. 2B), for example by sequencing the entire NPPF amplicon, or a portion thereof (such as an amount sufficient to permit identification of the modified or unmodified nucleic acid molecule previously hybridized to the NPPF). The disclosure is not limited to a particular sequencing method. In some examples, multiple different NPPF amplicons are sequenced in a single reaction. In one example, an experiment tag of the NPPF amplicon, which can be designed to correspond to a particular nucleic acid molecule, can be sequenced. Thus, if the 3′ end of the NPPF amplicon has a sequence at the terminal 2 to 25 nucleotides (such as the terminal 2 to 5 or 2 to 7, for example the terminal 2, 3, 4, 5, 6, 7, 8, 9, or 10 nucleotides) which represent a unique sequence for each nucleic acid molecule measured, then this is all of the NPPF amplicon that needs to be sequenced to identify the target, and by counting the number of such experiment tags sequenced, the amount of each modified nucleic acid molecule, unmodified nucleic acid molecule, or both, in the sample can be determined.

In some examples, the sequencing method used is chain termination sequencing, dye termination sequencing, pyrosequencing, nanopore sequencing, or massively parallel sequencing (also called next-generation sequencing (or NGS)), which is exemplified by ThermoFisher Ion Torrent™ sequencers (e.g. Ion Torrent Personal Genome Machine (PGM™, S5™, or Genexus™ systems), Illumina-branded NGS sequencers (e.g., MiSeq™, NextSeq™) (or as otherwise derived from Solexa™ sequencing) and 454 sequencing from Roche Life Sciences. In some examples, single molecule sequencing is used. In one example, the sequencing method uses bridge PCR (e.g., Illumina®). In one example, the Helicos® or PacBio® single molecule sequencing method is used.

In one example, the sequencing method uses sequencing-by-synthesis. In one example, the sequencing method uses sequencing-by-binding. In one example, the sequencing method uses single-molecule sequencing. In one example, the sequencing method uses semiconductor sequencing.

In one example, a next-generation sequencer (NGS) is used, such as those from Illumina®, Roche®, Genapsys, or Thermo Fisher Scientific®, for example, SOLiD®/Ion Torrent® S5 from Thermo Fisher Scientific®, NovaSeq/NextSeq/MiSeq from Illumina®, or GS FLX Titanium®/GS Junior® from Roche®.

In one example, a nanopore-type sequencer is used.

Sequencing adaptors (such as specific sequences or poly-A or poly T tails present on NPPF amplicons, for example as introduced using PCR) can be used for capture of the amplicons for sequencing on a particular platform.

Although sequencing by Ion Torrent® or Illumina® typically involves nucleic acid preparation, accomplished by random fragmentation of nucleic acid, followed by in vitro ligation of common adaptor sequences, for the disclosed methods, the step of random fragmentation of the nucleic acid to be sequenced can be eliminated, and the in vitro ligation of adaptor sequences is replaced by sequences present in the NPPF amplicon, such as an experiment tag present in the NPPF amplicon or a sequencing adaptor sequence present in the NPPF or added to the NPPF amplicon during amplification. For some sequencing methods, a sequencing primer is hybridized to the amplicons after amplification on the sequencing chip/bead amplicon.

It will be appreciated that the NPPF can be designed for sequencing by any method, on any sequencer developed currently or in the future. The NPPF itself does not limit the method of sequencing used, nor the enzyme used. Other methods of sequencing are or will be developed, and one skilled in the art can appreciate that the generated NPPF amplicons (or nucleic acid molecule hybridized to the NPPF) will be suitable for sequencing on these systems.

### E. Controls

In some embodiments, the method includes the use of one or more positive and/or negative controls subject to the same reaction conditions as described herein. The use of tagging of NPPFs permits actual different samples to be used as controls but processed for sequencing and run in the same sequencing lane as test samples. DNA can be measured as a control for the number of cells when measuring target RNA.

In some embodiments, the method includes the use of one or more positive controls, one or more negative controls, or combinations thereof, subjected to the same reaction conditions as the test sample. Exemplary controls can include, for example, a positive control modified RNA or modified DNA, such as an in vitro transcript generated using a modified base, or a RNA or DNA oligonucleotide with a modified base, and a negative control unmodified RNA or DNA. NPPFs used to detect these controls are used within the pool of NPPFs for the test sample. The ratio of signal from the positive to the negative control elements may then be used to determine per-reaction signal-to-noise or specificity of the enrichment of modified nucleic acids.

In some examples, a “positive control” includes an internal quantitation control for variables such as the number of cells lysed for each sample, the recovery of DNA or RNA, or the expression level of a RNA, such as an ERCC Spike in Control Mix (ThermoFisher Scientific), which contains several RNAs at known quantitates and known ratios to one another, along with one or more NPPFs, CFSs and the like, which are specific for these spike-in RNAs. Inclusion of these controls allows for a quantitation standard curve to be generated within each sample. This allows for comparison between samples, and between enriched and non-enriched samples to determine percent modification of a given transcript.

In some examples, a “positive control” includes an internal normalization control for variables such as the number of cells lysed for each sample, the recovery of DNA or RNA, or the hybridization efficiency, such as one or more NPPFs, CFSs, and the like, which are specific for one or more basal level or constitutive housekeeping genes, such as structural genes (e.g., actin, tubulin, or others) or DNA binding proteins (e.g., transcription regulation factors, or others). In some examples, a positive control includes glyceraldehyde-3-phosphate dehydrogenase (GAPDH), peptidylproylyl isomerase A (PPIA), large ribosomal protein (RPLP0), ribosomal protein L19 (RPL19), or other housekeeping genes discussed below. Other positive controls can be spiked into the sample to control for the assay process, independent of sample.

In other examples, a positive control includes an NPPF specific for a modified (or unmodified) DNA or RNA known to be present in the sample (for example a modified nucleic acid sequence likely to be present in the sample being tested). For example, the corresponding positive control NPPF can be added to the reaction prior to or during hybridization with the plurality of test NPPFs.

In some examples, a positive control includes a nucleic acid molecule known to be present in the sample (for example a modified nucleic acid sequence likely to be present in the sample being tested). The corresponding positive control nucleic acid molecule (such as in vitro transcribed nucleic acid or nucleic acid isolated from an unrelated sample) can be added to the reaction prior to or during hybridization with the plurality of NPPFs.

In some examples, a “negative control” includes one or more NPPFs, CFSs, or the like, whose complement is known not to be present in the sample, for example as a control for hybridization specificity, such as a nucleic acid sequence from a species other than that being tested, e.g., a plant nucleic acid sequence when human nucleic acids are being analyzed (for example, Arabidopsis thaliana AP2-like ethylene-responsive transcription factor (ANT)), or a nucleic acid sequence not found in nature.

In some examples, a “negative control” includes one or more NPPFs, CFSs, or the like, targeting a nucleic acid that is known not to be modified within the sample being tested.

### V. Nuclease Protection Probes with Flanking Sequences (NPPFs)

The disclosed methods permit sequencing of one or more modified (and in some examples also unmodified) nucleic acid molecules, for example simultaneously or contemporaneously. Based on the sequence of nucleic acids in a sample, NPPFs can be designed for use in the disclosed methods using the criteria set forth herein in combination with the knowledge of one skilled in the art. In some examples, the disclosed methods include generation of one or more appropriate NPPFs for detection of particular targets. Each NPPF, under a variety of conditions (known or empirically determined), specifically hybridizes (or is capable of specifically hybridizing) to a particular nucleic acid sequence or portion thereof, if such is present in the tested sample. In one example, a plurality of NPPFs are used simultaneously, such as the HTG Transcriptome Panel that can detect 19,398 different human mRNA molecules.

The NPPFs include a region that is complementary to a nucleic acid molecule known or suspected to be a sample, such that for each particular nucleic acid sequence of interest, there is at least one NPPF in the reaction that is specific for the target modified nucleic acid sequence. For example, if there are 2, 3, 4, 5, 6, 7, 8, 9 or 10 different modified nucleic acid sequences to be sequenced, the method will correspondingly use at least 2, 3, 4, 5, 6, 7, 8, 9 or 10 different NPPFs (wherein each NPPF corresponds to a particular nucleic acid sequence). Thus, in some examples, the methods use at least two NPPFs, wherein each NPPF is specific for a different modified (or unmodified) nucleic acid molecule. However, one will appreciate that several different NPPFs can be generated to a particular nucleic acid molecule, such as many different regions of a single modified nucleic acid sequence. In one example, an NPPF includes a region that is complementary to a sequence found only in a single gene in the transcriptome. NPPFs are designed to be specific for a modified or unmodified nucleic acid molecule and to have similar Tₘ's (if to be used in the same reaction).

Thus, a single sample may be contacted with one or more NPPFs. A set of NPPFs is a collection of two or more NPPFs each specific for a different target and/or a different portion of a same target. A set of NPPFs can include at least, up to, or exactly 2, 3, 4, 5, 6, 7, 8, 9, 10, 12, 15, 20, 25, 30, 50, 100, 500, 1000, 2000, 3000, 5000, 10,000, 15,000, 19,000, 19,398, 19,500, 20,000, 30,000, 40,000, 50,000, or 100,00 different NPPFs. In some examples, a sample is contacted with a sufficient amount of NPPF to be in excess of the corresponding nucleic acid molecule for such NPPF, such as a 100-fold, 500-fold, 1000-fold, 10,000-fold, 100,000-fold or 10⁶-fold excess. In some examples, if a set of NPPFs is used, each NPPF of the set can be provided in excess to its respective target (or portion of a target) in the sample. Excess NPPF can facilitate quantitation of the amount of NPPF that binds a particular nucleic acid molecule. Some method embodiments involve a plurality of samples (e.g., at least, up to, or exactly 10, 25, 50, 75, 96, 100, 500, 1000, 2000, 3000, 5000 or 10,000 different samples) simultaneously or contemporaneously contacted with the same NPPF or set of NPPFs.

FIG. 1 shows an exemplary NPPF 100 having a region 102 that includes a sequence that specifically binds to or hybridizes to corresponding nucleic acid molecule (such as a modified or unmodified nucleic acid molecule), as well as flanking sequences 104, 106 at the 5′- and 3′-end of the NPPF, wherein the flanking sequences bind or hybridize to their complementary sequences (referred to herein as CFSs). The NPPFs (as well as CFSs that bind to the NPPFs) can be composed of natural (such as ribonucleotides (RNA), or deoxyribonucleotides (DNA)) or unnatural nucleotides (such as locked nucleic acids (LNAs, see, e.g., U.S. Pat. No. 6,794,499), peptide nucleic acids (PNAs)), and the like. The NPPFs can be single- or double-stranded. In some examples, the NPPFs include one or more synthetic bases or alternative bases (such as inosine). Modified nucleotides, unnatural nucleotides, synthetic, or alternative nucleotides can be used in NPPFs at one or more positions (such as 1, 2, 3, 4, 5, or more positions). In some examples, use of one or more modified or unnatural nucleotides in the NPPF can increase the Tₘ of the NPPF relative to the Tₘ of a NPPF of the same length and composition which does not include the modified nucleic acid. One of skill in the art can design probes including such modified nucleotides to obtain a probe with a desired Tₘ. In one example, an NPPF is composed of DNA or RNA, such as single stranded (ssDNA) or branched DNA (bDNA). In one example, an NPPF is an aptamer.

Methods of empirically determining the appropriate size of a NPPF for use with particular nucleic acid molecule or samples (such as fixed or crosslinked samples) are routine. In specific embodiments, a NPPF can be up to 500 nucleotides in length, such as up to 400, up to 250, up to 100, or up to 75 nucleotides in length, including, for example, in the range of 20-500, 20-250, 25-200, 25-100, 25-75, or 25-50 nucleotides in length. In one non-limiting example, an NPPF is at least 35 nucleotides in length, such as at least 40, at least 45, at least 50, at least 75, at least 100, at least 150, or at least 200 nucleotides in length, such as 50 to 200, 50 to 100 or 75 to 200, or 36, 72, or 100 nucleotides in length. Particular NPPF embodiments may be longer or shorter depending on desired functionality.

The sequence 102 that specifically binds to a nucleic acid molecule (such as a modified nucleic acid sequence) is complementary in sequence to the nucleic acid sequence whose sequence us to be determined, but may or may not hybridize to bases that are modified. One skilled in the art will appreciate that the sequence 102 need not be complementary to an entire corresponding nucleic acid molecule (e.g., if the corresponding nucleic acid molecule is a gene of 100,000 nucleotides, the sequence 102 can be a portion of that, such as at least 10, at least 15, at least 20, at least 25, at least 30, at least 40, at least 50, at least 100, or more consecutive nucleotides complementary to a particular nucleic acid molecule). The specificity of a probe increases with length. Thus for example, a sequence 102 that specifically binds to a region of the modified (or unmodified) nucleic acid sequence which includes 25 consecutive nucleotides will anneal to a nucleic acid molecule with a higher specificity than a corresponding sequence of only 15 nucleotides. Thus, the NPPFs disclosed herein can have a sequence 102 that specifically binds to a nucleic acid sequence which includes at least 10, at least 15, at least 20, at least 25, at least 30, at least 40, at least 50, at least 100, or more consecutive nucleotides complementary to a particular nucleic acid molecule (such as about 6 to 50, 10 to 40, 10 to 60, 15 to 30, 18 to 23, 19 to 22, or 20 to 25 consecutive nucleotides complementary to a modified or unmodified DNA or a modified or unmodified RNA, but may or may not hybridize to bases that are modified). Particular lengths of sequence 102 that specifically binds to the nucleic acid sequence that can be part of the NPPFs used to practice the methods of the present disclosure include 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides complementary to a nucleic acid molecule. In some examples where the modified or unmodified nucleic acid molecule is an miRNA (or siRNA), the length of the sequence 102 that specifically binds to the modified or unmodified nucleic acid sequence can be shorter, such as 20-30 nucleotides in length (such as 20, 21, 22, 23, 24, 25, 26, 27, 28 29, or 30 nucleotides) to match the miRNA (or siRNA) length. However, one skilled in the art will appreciate that the sequence 102 that specifically binds to the corresponding nucleic acid molecule to be sequenced need not be 100% complementary to its corresponding nucleic acid molecule. Depending on the reaction conditions and the corresponding selectivity of the nuclease used, more than one mismatch may be required (such as at least two adjacent mismatches) for nuclease digestion to occur. In some examples, the NPPF is degenerate at one or more positions (such as 1, 2, 3, 4, 5, or more positions), for example, a mixture of nucleotides (such as 2, 3, or 4 nucleotides) at a specified position in the sequence 104 that specifically binds to its corresponding nucleic acid molecule.

The sequence of the flanking sequence 104, 106 can provide a universal amplification point that is complementary to at least a portion of an amplification primer. The flanking sequence thus permits multiplexing, as the same amplification primers can be used to amplify NPPFs specific for different nucleic acid molecules whose sequence is to be determined. The flanking sequence is not similar to a sequence found in the genome present in the sample tested. For example, if the nucleic acid molecules whose sequence is to be determined are human, the sequence of the flanking sequence is not similar to a sequence found in the human genome. This helps to reduce binding of non-corresponding sequences that may be present in the target genome from binding to the NPPFs. Methods of analyzing a sequence for its similarity to a genome can be used.

The flanking sequence 104, 106 can also be used to permit capture of an NPPF amplicon, for example capture to a substrate. For example, an NPPF containing a flanking sequence that includes a sequence complementary to a nucleic acid capture probe present on a surface (such as directly conjugated to a surface), can hybridize to the nucleic acid capture probe permitting capture or binding of the NPPF amplicon to the surface. Thus, in some examples, the flanking sequence includes (or permits addition of, for example during amplification) of an experimental tag, such as one that permits capture of the NPPF amplicon. One will appreciate that other experimental tags can be used, such as those used to uniquely identify an NPPF or populations of NPPFs, and that such experimental tags can be part of the NPPF, or can be added later, for example by using a primer complementary to the flanking sequence and which also includes a sequence complementary to the tag to be added to the resulting amplicon. The flanking sequences also permit labeling of the NPPF, for example during amplification of the NPPF, or by using a labeled probe that is complementary to the flanking sequence, and allowing the probe to bind to the NPPF. In some examples, the flanking sequence includes (or permits addition of, for example during amplification) of a sequencing adapter, such as a poly-A or poly-T sequence needed for some sequencing platforms.

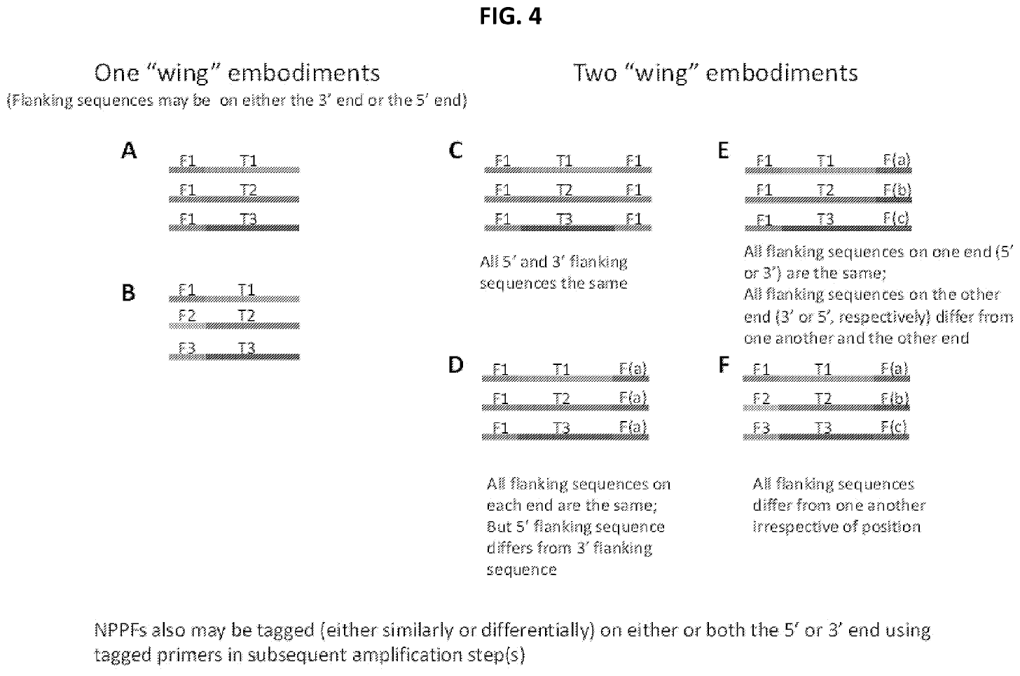

One will appreciate than an NPPF can include one or two flanking sequences (e.g., one at the 5′-end, one at the 3′-end, or both), and that the flanking sequences can be the same or different. As illustrated in FIGS. 4A and 4B, the NPPF can include a single flanking sequence. FIGS. 4A and 4B show the flanking sequence at the 5′-end, but one will appreciate it can also be at the 3′-end instead. FIG. 4A shows an example where all of the NPPFs in the reaction have the same flanking sequence F1. Amplification with an F1-specific primer (such as a labeled primer) could be used to add the same 5′- or 3′-tag (e.g., sequencing adaptor or experimental tag) to each NPPF. For example, the same sequencing adapter could be added to all of the NPPFs, permitting sequencing of the NPPFs in the same sequencing platform. FIG. 4B shows an example where each NPPF (or each subpopulation of NPPFs) in the reaction have a different flanking sequence, F1 to F3. For example, F1, F2, and F3 could be complementary to a capture nucleic acid probe 1, 2, and 3, respectively on a surface. In another example, amplification with T1-F1-, T2-F2-, and T3-F3-specific primers can be used to add a different experimental tag to each different NPPF (or populations of NPPFs).

As illustrated in FIGS. 4C-4F, the NPPF can in some examples include two flanking sequences, one at the 5′-end the other at the 3′-end of the NPPF. FIG. 4C shows an example where all of the NPPFs in the reaction have the same flanking sequence, F1, at both ends. FIG. 4D shows an example wherein all of the flanking sequences on the 5′-end are the same (e.g., F1), and all of the flanking sequences on the 3′-end are the same (e.g., F(a)), but the 5′-end and 3′-end flanking sequences differ. In such an example, this permits the inclusion of for example of the same experimental tag on one end of the NPPFs, and the inclusion of for example of the same sequencing adaptor to the other side of the NPPFs. As there will be no primer hybridization bias each NPPF should be tagged with the same fidelity. FIG. 4E shows an example wherein all of the flanking sequences on one end are the same (e.g., F1 on the 5′-end), but all of the flanking sequences on the other end differ from one another (e.g., F(a), F(b), and F(c)). In such an example, this permits the use of a single capture probe to capture all of the NPPFs (e.g., using a capture probe having at least a portion of its sequence complementary to F1). The flanking sequences on the other end, F(a), F(b) and F(c), could be used for example to differentially label each NPPF (such as using different experiment tags). Alternatively, F(a), F(b) and F(c) could be complementary to capture probes 1, 2, and 3, respectively, and F1 could be used to label all of the NPPFs in the same way. FIG. 4F shows an example wherein all of the flanking sequences are different, irrespective of their position (e.g., F(a), F(b), F(c), F1, F2, and F3). In this example, each flanking sequence can be used for a different experiment tag or for combinations of different experiment tags and different sequencing adapters.

Thus, an NPPF sequence can be represented by 1-2-3 where 1 and 3 are flanking sequences on either side of sequence 2 (which is complementary to a nucleic acid known or suspected to be in the sample tested). Each of these regions can hybridized at some point in the method to its complementary sequence. For example, A can be complementary to flanking sequence 1 of the NPPF (e.g., A can be a CFS complementary to sequence 1), B can be complementary to sequence 2 of the NPPF (e.g., a modified sequence complementary to sequence 2), and C can be complementary to the flanking sequence 3 of the NPPF (e.g., C can be a CFS complementary to sequence 3). This is what occurs during the hybridization of the modified or unmodified nucleic acid molecules and CFSs, to their corresponding NPPF. For example:
1-2-3
A-B-C

In some examples, the experimental tags (such as those that distinguish experiments or patients from one another) and sequencing adapters, represented by D and E respectively, are added using the flanking sequences, for example during amplification (such that the amplification primer is complementary to the flanking sequence and includes a sequence complementary to the tag or adapter to be added to the resulting NPPF amplicon). For example, amplification of the NPPF with such primers would result in a sequence as follows: E-1-2-3-D or D-1-2-3-E.

Table 1 shows five exemplary combinations of 5′-tags (such as experimental tags or sequencing adapters), 5′-flanking sequences, sequences that hybridize to a nucleic acid molecule whose sequence is to be determined, 3-flanking sequences, and 3-tags. The 5′-tags and 3′-tags are added during amplification. The 5-flanking sequences and 3′-flanking sequences are sequences that are part of the original NPPF (and thus part of the flanking sequence itself).

TABLE 1
Five Exemplary Combinations

| | | | | |
|---|---|---|---|---|
| | | Region that | | |
| | | hybridizes to | | |
| | | nucleic acid | | |
| | 5′-Flanking | molecule to | 3′-Flanking | |
| 5′-Tag | Sequence | be sequenced | Sequence | 3′-Tag |

| | | | | |
|---|---|---|---|---|
| Ex. 1 | None | Sequencer | Sequencer | None |
| | | Adapter | Adapter | |
| Ex. 2 | Sequencing | Sequence- | Sequence- | Sequencing |
| | Adapter | specific | specific | Adapter |
| | | identifier | identifier | |
| Ex. 3 | Experimental | Experimental | Experimental | Experimental |
| | tag (short | tag (short | tag (short | tag (short |
| | sequence or | sequence or | sequence or | sequence or |
| | modified bases, | modified bases, | modified bases, | modified bases, |
| | identifier for | identifier for | identifier for | identifier for |
| | one/several | one/several | one/several | one/several |
| | reactions to be | reactions to be | reactions to be | reactions to be |
| | independently | independently | independently | independently |
| | discerned: by | discerned: by | discerned: by | discerned: by |
| | (i.e.) patient, | (i.e.) patient, | (i.e.) patient, | (i.e.) patient, |
| | sample, cell | sample, cell | sample, cell | sample, cell |
| | type, time | type, time | type, time | type, time |
| | course timepoint, | course timepoint, | course timepoint, | course timepoint, |
| | treatment) | treatment) | treatment) | treatment) |
| Ex. 4 | Biotin or | Biotin or | Biotin or | Biotin or |
| | other detection | other detection | other detection | other detection |
| | (e.g., hapten) | (e.g., hapten) | (e.g., hapten) | (e.g., hapten) |
| | tag/capture | tag/capture | tag/capture | tag/capture |
| | sequence | sequence | sequence | sequence |
| Ex. 5 | Site for | Site for | Site for | Site for |
| | cleavage | cleavage | cleavage | cleavage |
| | (enzymatic/ | (enzymatic/ | (enzymatic/ | (enzymatic/ |
| | modified base) | modified base) | modified base) | modified base) |
| | | “Buffer” (e.g., | “Buffer” (e.g., | |
| | | spacer or | spacer or | |
| | | universal) | universal) | |
| | | sequence | sequence | |

In specific examples, each flanking sequence does not specifically bind to any other NPPF sequence (e.g., sequence 102 or other flanking sequence) or to any component of the sample. In some examples, if there are two flanking sequences, the sequence of each flanking sequence 104, 106 is different. Ideally, if there are two different flanking sequences (for example two different flanking sequences on the same NPPF and/or to flanking sequences of other NPPFs in a set of NPPFs), each flanking sequence 104, 106 has a similar melting temperature (Tₘ), such as a Tₘ+/−about 10° C. or +/−5° C. of one another, such as +/−4° C., 3° C., 2° C., or 1° C.

In particular examples, the flanking sequence 104, 106 is at least 12 nucleotides in length, such as at least 15, at least 20, at least 25, at least 30, at least 40, or at least 50 nucleotides in length, such as 12-50 or 12-30 nucleotides, for example, 20, 21, 22, 23, 24, 25, 26, 27, 28, 29 or 30 nucleotides in length, wherein the contiguous nucleotides not found in a nucleic acid molecule present in the sample to be tested. The flanking sequences are protected from degradation by the nuclease by hybridizing molecules to the flanking sequences which have a sequence complementary to the flanking sequences (CFSs).

Factors that affect NPPF-target and NPPF-CFS hybridization specificity include length of the NPPF and CFS, melting temperature, self-complementarity, and the presence of repetitive or non-unique sequence. See, e.g., Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. Conditions resulting in particular degrees of hybridization (stringency) will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. In some examples, the NPPFs utilized in the disclosed methods have a Tₘ of at least about 37° C., at least about 42° C., at least about 45° C., at least about 50° C., at least about 55° C., at least about 60° C., at least about 65° C., at least about 70° C., at least about 75° C., at least about 80° C., such as about 42° C.-80° C. (for example, about 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.). In one non-limiting example, the NPPFs utilized in the disclosed methods have a Tₘ of about 42° C. The Tₘ of a probe can be determined (see e.g., Sambrook et al., Molecular Cloning: A Laboratory Manual, 3d ed., Cold Spring Harbor Press, 2001, Chapter 10). In some examples, the NPPFs for a particular reaction are selected to each have the same or a similar Tₘ in order to facilitate simultaneous detection or sequencing of multiple modified (and in some examples also unmodified) nucleic acid molecules in a sample, such as Tₘs+/−about 10° C. of one another, such as +/−10° C., 9° C., 8° C., 7° C., 6° C., 5° C., 4° C., 3° C., 2° C., or 1° C. of one another.

### A. Flanking Sequences

One or both of the flanking sequences of the NPP (e.g., 104 or 106 of FIG. 1) include a sequence that provides a universal amplification point. Such a sequence is complimentary to at least a portion of an amplification primer. This allows the primer to hybridize to the NPPF, and amplify the NPPF. As flanking sequences can be identical between NPPFs specific for different target modified nucleic acid molecules, this permits the same primer to be used to amplify any number of different NPPFs. For example, an NPPF can include a 5′-flanking sequence, and a 3′-flanking sequence, wherein the 5′- and the 3′-flanking sequences are different from one another, but are the same for a plurality of NPPFs for different nucleic acid molecules whose sequence is to be determined. Thus an amplification primer that includes a sequence complementary to the 5′-flanking sequence, and an amplification primer that includes a sequence complementary to the 3-flanking sequence, can both be used in a single reaction to amplify multiple NPPFs, even if the NPPFs are specific for different nucleic acid molecules whose sequence is to be determined.

In some examples, the flanking sequence does not include an experiment tag sequence and/or a sequencing adapter sequence. In some examples, a flanking sequence includes or consists of an experiment tag sequence and/or sequencing adapter sequence. In other examples, the primers used to amplify the NPPFs include an experiment tag sequence and/or sequencing adapter sequence, thus permitting incorporation of the experiment tag and/or sequencing adapter into the NPPF amplicon during amplification of the NPPF.

In one example, a flanking sequence is designed such that the sequence forms a loop on itself. Thus, one region of a flanking sequence is complementary to a second region of the same flanking sequence, such that the first and second regions hybridize to one another, forming a loop or hairpin. This would eliminate the need for CFSs, as the second region would protect the first region during the nuclease step.

B. Primers that Bind the Flanking Sequences

The amplification primers that specifically bind or hybridize to the flanking sequences can be used to initiate amplification, such as PCR amplification. In addition, the amplification primers can be used to introduce nucleic acid tags (such as experiment tags or sequencing adapters) and/or detectable labels to NPPFs. For example, in addition to the amplification primer having a region complementary to the flanking sequence, it can also include a second region having a nucleic acid sequence that results in addition of an experiment tag, sequencing adapter, detectable label, or combinations thereof, to the resulting NPPF amplicon. An experiment tag or sequencing adapter can be introduced at the NPPF 5′- and/or 3′-end. In some examples, two or more experiment tags and/or sequencing adaptors are added to a single end or both ends of the NPPF amplicon, for example using a single primer having a nucleic acid sequence that results in addition of two or more experiment tags and/or sequencing adapters. Experiment tags can be used, for example, to differentiate one sample or sequence from another, or to permit capture of an NPPF amplicon by a substrate. Sequence tags permit capture of the resulting NPPF amplicon by a particular sequencing platform.

A detectable label can be introduced at any point of the NPPF, including the 5′- and/or 3′-end. In one example, the label is introduced to an NPPF amplicon by hybridization of a labeled probe complementary to the NPPF amplicon. In one example, the label is introduced to an NPPF amplicon by use of a labeled primer during amplification of the NPPF, thereby generating a labeled NPPF amplicon. Detectable labels permit detection of the NPPF amplicons.

In some examples, such primers are at least 12 nucleotides in length, such as at least 15, at least 20, at least 30, at least 40 or at least 50 nucleotides (for example 25 nucleotides). In some examples the primers include a detectable label (and such primers can be referred to as probes), such as biotin, that gets incorporated into the NPPF amplicons.

### C. Addition of Experiment Tags

Experimental tags can be part of the NPPF when generated (for example be part of the flanking sequence). In another example, the experiment tag is added later, for example during amplification of the NPPF, resulting in an NPPF amplicon containing an experimental tag. The presence of the universal flanking sequences on the NPPF permit the use of universal primers, which can introduce other sequences onto the NPPFs, for example during amplification.

Experiment tags, such as one that differentiates one sample from another, can be used to identify the particular target modified sequence associated with the NPPF, or permit capture of an NPPF amplicon by a substrate (wherein the experiment tag is complementary to a capture probe on the substrate, permitting hybridization between the two). In one example, the experiment tag is the first three, five, ten, twenty, or thirty nucleotides of the 5′- and/or 3′-end of the NPPF or NPPF amplicon.

In one example an experiment tag is used to differentiate one sample from another. For example, such a sequence can function as a barcode, to allow one to correlate a particular sequence detected with a particular sample, patient, or experiment (such as a particular reaction well, day or set of reaction conditions). This permits a particular NPPF that is detected or sequenced to be associated with a particular subject or sample or experiment for instance. The use of such tags provides a way to lower cost per sample and increase sample throughput, as multiple NPPF amplicons can be tagged and then combined (for example from different experiments or patients), for example in a single sequencing run or detection array. This allows for the ability to combine different experimental or subject samples into a single run, within the same instrument channel. For example, such tags permitting 100s, 1,000s, or 10,000s of different experiments to be sequenced in a single run, within a single channel. For example, pooling 100 samples per channel, 8,000 samples can be tested in a single run of an 8-channel sequencer. In addition, if the method includes the step of gel purifying the completed amplification reaction (or other method of purification or clean up that does not require actual separation) only one gel (or clean up or purification reaction or process) is needed to be run per detection or sequencing run. The sequenced NPPF amplicons can then be sorted, for example by the experiment tags.

In one example the experiment tag is used to identify the particular nucleic acid molecules associated with the NPPF. In this case, using an experimental tag to correspond to a particular sequence can shorten the time or amount of sequencing needed, as sequencing the end of the NPPF instead of the entire NPPF can be sufficient. For example, if such an experiment tag is present on the 3-end of the NPPF amplicon, the entire NPPF amplicon sequence itself does not have to be sequenced to identify the nucleic acid molecule which hybridized to the NPPF. Instead, only the 3′-end of the NPPF amplicon containing the experiment tag needs to be sequenced. This can significantly reduce sequencing time and resources, as less material needs to be sequenced.

In one example the experiment tag is used to permit capture NPPFs, such as to concentrate NPPFs or NPPF amplicons from a sample. For example, the experiment tag can have a sequence that is complementary to the sequence of at least a portion of a capture probe on a substrate surface, thereby permitting hybridization of the NPPF to the capture probe. For instance, following amplification, NPPF amplicons containing an experimental tag (such as a population of NPPF amplicons containing the same experimental tag) can be isolated from other materials by incubating the sample with a substrate (such as magnetic beads) containing a plurality of capture probes with sequences complementary to the experimental tag. After their capture, the NPPF amplicons can be detected or sequenced, or can be released from the substrate for further analysis. In one example, the substrate is magnetic beads, and the PCR reaction containing NPPF amplicons is incubated with the beads. The beads are then held in a magnetic field while the sample solution (containing non-desired nucleic acid molecules and other materials) is removed. The captured NPPFs can be eluted into a smaller volume by reversing hybridization, such as by addition of base and heating. One will appreciate that similar methods can be used with other NPPFs and other substrates (such as by using a solid substrate and a flow through device), resulting in the captured NPPFs being eluted into a smaller volume. If a hapten is added during amplification, it can be used for capture. One advantage of such a method is that the NPPFs or NPPF amplicons can be isolated from a large sample, such as 1 ml plasma, and eluted into a smaller volume used for assays, such as 20 μl.

Experimental tags can also be used for amplification, such as nested amplification, or two stage amplification.

In particular examples, the experiment tag is at least 3 nucleotides in length, such as at least 5, at least 10, at least 15, at least 20, at least 25, at least 30, at least 40, or at least 50 nucleotides in length, such as 3-50, 3-20, 12-50 or 12-30 nucleotides, for example, 3, 5, 6, 8, 10, 20, 21, 22, 23, 24, 25, 26, 27, 28, 29 or 30 nucleotides in length.

### D. Addition of Sequencing Adapters

Sequencing adapters can be part of the NPPF when generated (for example be part of the flanking sequence). In another example, the sequencing adapter is added later, for example during amplification of the NPPF, resulting in an NPPF amplicon containing a sequencing adapter. The presence of the universal flanking sequences on the NPPF permit the use of universal primers, which can introduce other sequences onto the NPPFs, for example during amplification.

A sequencing adapter can be used add a sequence to an NPPF amplicon needed for a particular sequencing platform. For example, some sequencing platforms (such as the 454 and Illumina platforms) require the nucleic acid molecule to be sequenced to include a particular sequence at its 5′- and/or 3′-end, for example to capture the molecule to be sequenced. For example, the appropriate sequencing adapter is recognized by a complementary sequence on the sequencing chip, flowcell, or beads, and the NPPF captured by the presence of the sequencing adapter.

In one example, a poly-A (or poly-T), such as a poly-A or poly-T at least 10 nucleotides in length is added to the NPPF during PCR amplification. In a specific example, the poly-A (or poly-T) is added to the 3′-end of the NPPF. In some examples, this added sequence is poly-adenylated at its 3′ end using a terminal deoxynucleotidyl transferase (TdT).

In particular examples, the sequencing tag added is at least 12 nucleotides (nt) in length, such as at least 15, at least 20, at least 25, at least 30, at least 40, or at least 50 nt in length, such as 12-50 or 12-30 nt, for example, 20, 21, 22, 23, 24, 25, 26, 27, 28, 29 or 30 nt in length.

### E. Detectable Labels

In some examples, the disclosed NPPFs, PCR primers, or both, include one or more detectable labels. A detectable label is a molecule or material that can be used to produce a detectable signal that indicates the presence or concentration of an NPPF or NPPF amplicon (e.g., the bound or hybridized probe) in a sample. Thus, a labeled NPPF provides an indicator of the presence or concentration of a nucleic acid molecule whose sequence is to be determined (e.g., a modified DNA or a modified RNA) in a sample. The disclosure is not limited to the use of particular labels, although examples are provided.

In some examples, the label is incorporated into the NPPF during synthesis of the NPPF. In some examples, the label is incorporated into the NPPF during amplification, for example using labeled primers (thus generating labeled NPPF amplicons). In yet other examples, the NPPF is labeled by using a labeled probe that is complementary to, and thus hybridizes to, a portion of the NPPF (such as an NPPF amplicon), such as a flanking region of the NPPF.

In some examples, each of the NPPFs included in a plurality of NPPFs utilized in the disclosed methods are labeled with the same detectable label. In other examples at least one NPPF is labeled with a different detectable label than at least one other NPPF in the plurality of NPPs. For example, at least one NPPF included in the plurality of NPPFs can be labeled with a fluorophore (such as Cy-3™) and at least one NPPF included in the plurality of NPPs can be labeled with a different fluorophore (such as Cy-5™). In some examples, the plurality of NPPFs can include at least 2, 3, 4, 5, 6, or more different detectable labels. Similarly, amplification primers used in the methods provided herein can be labeled with the same or different detectable labels.

A label associated with one or more nucleic acid molecules (such as an NPPF or amplification primer) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, electroluminescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens, and paramagnetic and magnetic molecules or materials. Additional detectable labels include Raman (light scattering) labels (e.g., Nanoplex® biotags, Oxonica, Bucks, UK). Other exemplary detectable labels include digoxin, the use of energy transfer and energy quenching pairs (such as FRET), IR, and absorbance/colorimetric labels.

In non-limiting examples, NPPFs or primers are labeled with dNTPs covalently attached to hapten molecules (such as a nitro-aromatic compound (e.g., dinitrophenyl (DNP)), biotin, fluorescein, digoxigenin, etc.). Haptens and other labels can be conjugated to dNTPs (e.g., to facilitate incorporation into labeled probes) (e.g., methods in U.S. Pat. Nos. 5,258,507, 4,772,691, 5,328,824, and 4,711,955). A label can be directly or indirectly attached to a dNTP at any location on the dNTP, such as a phosphate (e.g., α, β or γ phosphate) or a sugar. In some examples, detection of labeled nucleic acid molecules can be accomplished by contacting the hapten-labeled NPPF with a primary anti-hapten antibody. In one example, the primary anti-hapten antibody (such as a mouse anti-hapten antibody) is directly labeled with an enzyme. In another example, a secondary anti-antibody (such as a goat anti-mouse IgG antibody) conjugated to an enzyme is used for signal amplification. In other examples, the hapten is biotin and is detected by contacting the hapten-labeled NPPF with avidin or streptavidin conjugated to an enzyme, such as horseradish peroxidase (HRP) or alkaline phosphatase (AP).

Additional examples of detectable labels include fluorescent molecules (or fluorochromes). Exemplary fluorochromes are available, for example from Life Technologies, e.g., see, The Handbook—A Guide to Fluorescent Probes and Labeling Technologies. Examples of particular fluorophores that can be attached (for example, chemically conjugated) to a nucleic acid molecule (such as an NPPF) are provided in U.S. Pat. No. 5,866,366 to Nazarenko et al., such as 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); 2′, 7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores can also be used, for example those available from Life Technologies (Invitrogen; Molecular Probes (Eugene, OR)) and including the ALEXA FLUOR® series of dyes (for example, as described in U.S. Pat. Nos. 5,696,157, 6,130,101 and 6, 716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, OR); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the bandgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:2013-2016, 1998; Chan et al., Science 281:2016-2018, 1998; and U.S. Pat. No. 6,274,323.

Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlsbad, CA).

Additional labels include, for example, radioisotopes (such as ³H), metal chelates such as DOTA and DPTA chelates of radioactive or paramagnetic metal ions like Gd³⁺, and liposomes.

Detectable labels that can be used with nucleic acid molecules (such as an NPPF or amplification primer) also include enzymes, for example HRP, AP, acid phosphatase, glucose oxidase, β-galactosidase, β-glucuronidase, or β-lactamase. Where the detectable label includes an enzyme, a chromogen, fluorogenic compound, or luminogenic compound can be used in combination with the enzyme to generate a detectable signal (numerous of such compounds are commercially available, for example, from Life Technologies, Carlsbad, CA). Particular examples of chromogenic compounds include diaminobenzidine (DAB), 4-nitrophenylphosphate (pNPP), fast red, fast blue, bromochloroindolyl phosphate (BCIP), nitro blue tetrazolium (NBT), BCIP/NBT, AP Orange, AP blue, tetramethylbenzidine (TMB), 2,2′-azino-di-[3-ethylbenzothiazoline sulphonate](ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

Alternatively, an enzyme can be used in a metallographic detection scheme. Metallographic detection methods include using an enzyme, such as alkaline phosphatase, in combination with a water-soluble metal ion and a redox-inactive substrate of the enzyme. The substrate is converted to a redox-active agent by the enzyme, and the redox-active agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water-soluble metal ion, an oxidizing agent and a reducing agent, again to form a detectable precipitate. (See, for example, U.S. Pat. No. 6,670,113).

In some embodiments, the detectable label is attached to or incorporated in the NPPF or 5 primer at the 5′ end or the 3′ end (e.g., the NPPF or primer is an end-labeled probe). In other examples the detectable label is incorporated in the NPPF or primer at an internal position, such as 1, 2, 3, 4, 5, 6, 7, 8, 9, 10, 11, 12, 13, 14, 15, or more bases from the 5′ end of the NPPF or primer, or 1, 2, 3, 4, 5, 6, 7, 8, 9, 10, 11, 12, 13, 14, 15, or more bases from the 3′ end of the NPPF or primer.

In one example, one of the flanking regions of the NPPF contains an acceptor or emitter (such as an acceptor fluorophore), while the amplification primer complementary to the flanking region contains the converse (such as a donor fluorophore). Thus, the primer-NPPF duplex emits detectable signal, but single stranded primers, or single stranded NPPFs, do not. The appearance of signal is a measure of the amount of NPPF in the sample analyzed, and can be measured without separation of the labeled excess primers from the amplified adducts. Examples of FRET acceptor-donor pairs include FAM as a donor fluorophore for use with JOE, TAMRA, and ROX, 3-(C-carboxy-pentyl)-3′-ethyl-5,5′-dimethyloxacarbocyanine (CYA) can serve as a donor fluorophore for rhodamine derivatives (such as R6G, TAMRA, and ROX) which can be used as acceptor fluorophores. Grant et al. (Biosens Bioelectron. 16:231-7, 2001) provide particular examples of FRET pairs that can be used in the methods disclosed herein.

### VI. Samples

A sample is any collective comprising one or more nucleic acid molecules, such as a biological sample or biological specimen. A sample can be collected or obtained, for example from a mammalian subject. In some examples, a sample is an in vitro or ex vivo culture of cells, such as bacterial cells, plant cells, invertebrate cells, or mammalian cells (such as human cells), such as liver, pancreatic, kidney, skeletal muscle, cardiac muscle, lung, colon, prostate, ovary, cervix, breast, epithelial, blood, or neuron cells. In some examples, a sample is an in vitro or ex vivo culture of cancerous mammalian cells, such as human cancer cells, such as a cancer of the liver, pancreas, kidney, bone, lung, colon, breast, prostate, ovary, cervix, skin, thyroid, blood, or central nervous system. The samples of use in the disclosed methods can include any specimen that includes nucleic acid (such as genomic DNA, cDNA, viral DNA or RNA, rRNA, tRNA, mRNA, miRNA, other non coding RNAs, oligonucleotides, nucleic acid fragments, modified nucleic acids, synthetic nucleic acids, or the like). In one example, the sample includes unstable RNA. In some examples, the nucleic acid molecule to be sequenced is cross-linked in the sample (such as a cross-linked DNA, mRNA, miRNA, or vRNA) or is soluble in the sample. In some examples, the sample is a fixed sample (e.g., FFPE), such as a sample that includes an agent that causes target molecule cross-linking. In some examples, nucleic acid molecules in the sample whose sequence is to be determined are not extracted, solubilized, or both, prior to analysis with the disclosed methods (e.g., sequencing). In some examples, modified nucleic acid molecules in the sample whose sequence is to be determined are cross-linked. In some examples, modified nucleic acid molecules in the sample whose sequence is to be determined are extracted, solubilized, or both, and in some examples also captured and purified, prior to sequencing.

In some examples, the disclosed methods include obtaining the sample prior to analysis of the sample. In some examples, the sample is from a subject having a disease, such as a cancer, an autoimmune disease, a neurological disease, or diabetes, who has been exposed to or administered one or more test agents, for example to determine if the test agent produced on or off target effects on the modification or un-modification of a nucleic acid molecule.

In some examples, the disclosed methods include treating an in vitro or ex vivo culture of cells (such as bacterial cells, bird cells, fish cells, reptile cells, mammalian cells, plant cells, or invertebrate cells) with one or more test agents, such as a pharmaceutical agent for treating a disease, a mutagen, a pathogen, a stress condition, or the like, and isolating modified nucleic acids from the cells and sequencing the modified nucleic acids using the methods provided herein. In some examples, the sequence of unmodified and modified nucleic acid molecules in the sample are also determined, without capturing and isolating modified nucleic acid molecules, wherein the results of this analysis can be compared to the analysis of the sample wherein modified nucleic acid molecules in the sample were captured and isolated and then sequenced. In some examples, RNA in the sample is reverse transcribed prior to performing the methods provided herein. However, the disclosed methods do not require reverse transcription, as RNA is effectively converted into a complementary probe sequence through hybridization and nuclease activity. It is sometimes desirable to sequence RNA molecules rather than the gene sequences which encode the RNA, since RNA molecules are not necessarily co-linear with their DNA template. And some organisms are RNA, such as RNA viruses.

In some examples, the sample is lysed prior to determining the sequence of nucleic acid molecules therein, such as modified and unmodified nucleic acids. The lysis buffer inactivates enzymes and prevents the degradation of nucleic acids (e.g., RNA), but after a limited dilution into a hybridization dilution buffer it permits nuclease activity and facilitates hybridization with stringent specificity. A dilution buffer can be added to neutralize the inhibitory activity of the lysis and other buffers, such as inhibitory activity for other enzymes (e.g., polymerase). Alternatively, the composition of the lysis buffer and other buffers can be changed to a composition that is tolerated, for example by a polymerase.

In some examples, the methods include analyzing a plurality of samples simultaneously or contemporaneously. For example, the methods can analyze at least two different samples (for example from different patients) simultaneously or contemporaneously. In one example, the methods can detect or sequence at least two different target nucleic acid molecules (such as at least 10,000 or at least 19,000 different targets) in at least two different samples (such as at least 5, at least 10, at least 100, at least 500, at least 1000, or at least 10,000 different samples) simultaneously or contemporaneously.

Exemplary samples include, without limitation, cells, blood smears, cytocentrifuge preparations, cytology smears, bodily fluids (e.g., blood and fractions thereof such as serum and plasma, saliva, sputum, urine, spinal fluid, gastric fluid, sweat, semen, etc.), cytological smears, buccal cells, extracts of tissues, cells or organs, tissue biopsies (e.g., tumor biopsies), fine-needle aspirates, punch biopsies, circulating tumor cells, fresh tissue, frozen tissue, fixed tissue, fixed and wax- (e.g., paraffin-)embedded tissue, bone marrow, and/or tissue sections (e.g., cryostat tissue sections and/or paraffin-embedded tissue sections). In some examples, the sample is a cell lysate generated from any such samples. The biological sample may also be a laboratory research sample such as a cell culture sample or supernatant.

Samples, such as a tissue or cell sample, can be obtained from a subject. Exemplary samples may be obtained from normal cells or tissues, or from diseased cells or tissues, such as neoplastic cells or tissues. In particular examples, a biological sample includes a tumor sample, such as a cancer sample.

Exemplary neoplastic cells or tissues may be included in or isolated from solid tumors, including lung cancer (e.g., non-small cell lung cancer, such as lung squamous cell carcinoma), breast carcinomas (e.g. lobular and duct carcinomas), adrenocortical cancer, ameloblastoma, ampullary cancer, bladder cancer, bone cancer, cervical cancer, cholangioma, colorectal cancer, endometrial cancer, esophageal cancer, gastric cancer, glioma, granular call tumor, head and neck cancer, hepatocellular cancer, hydatiform mole, lymphoma, melanoma, mesothelioma, myeloma, neuroblastoma, oral cancer, osteochondroma, osteosarcoma, ovarian cancer, pancreatic cancer, pilomatricoma, prostate cancer, renal cell cancer, salivary gland tumor, soft tissue tumors, Spitz nevus, squamous cell cancer, teratoid cancer, and thyroid cancer. Exemplary neoplastic cells may also be included in or isolated from hematological cancers including leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, and myelodysplasia.

For example, a sample from a tumor that contains cellular material can be obtained by surgical excision of all or part of the tumor, by collecting a fine needle aspirate from the tumor, as well as other methods. In some examples, a tissue or cell sample is applied to a substrate and analyzed to determine presence of one or more modified (or unmodified) DNAs or RNAs. A solid support useful in a disclosed method need only bear the biological sample and, optionally, permit the convenient detection of components (e.g., proteins and/or nucleic acid sequences) in the sample. Exemplary supports include microscope slides (e.g., glass microscope slides or plastic microscope slides), coverslips (e.g., glass coverslips or plastic coverslips), tissue culture dishes, multi-well plates, membranes (e.g., nitrocellulose or polyvinylidene fluoride (PVDF)) or BIACORE™ chips.

The disclosed methods are sensitive and specific and allow the sequence of nucleic acid molecules in a sample containing even a limited number of cells to be determined. Samples that include small numbers of cells, such as less than 250,000 cells (for example less than 100,000, less than 50,000, less than 20,000, less than 10,000, less than 1,000, less than 500, less than 200, less than 100 cells, or less than 10 cells, include but are not limited to, FFPE samples, fine needle aspirates (such as those from lung, prostate, lymph, breast, or liver), punch biopsies, needle biopsies, small populations of (e.g., FACS) sorted cells or circulating tumor cells, lung aspirates, small numbers of laser captured or macrodissected cells or circulating tumor cells, exosomes and other subcellular particles, or body fluids (such as plasma, serum, spinal fluid, saliva, and breast aspirates). For example, a particular DNA or RNA can be detected in as few as 1000 cells (such as a sample including 1000 or more cells, such as 1000, 2000, 3000, 4000, 5000, 6000, 7000, 8000, 9000, 10,000, 15,000, 20,000, 50,000, or more cells). In some examples, expression of a particular DNA or RNA can be detected in about 1000 to 100,000 cells, for example about 1000 to 50,000, 1000 to 15,000, 1000 to 10,000, 1000 to 5000, 3000 to 50,000, 6000 to 30,000, or 10,000 to 50,000 cells). In some examples, expression of a particular DNA or RNA be detected in about 100 to 250,000 cells, for example about 100 to 100,000, 100 to 50,000, 100 to 10,000, 100 to 5000, 100 to 500, 100 to 200, or 100 to 150 cells. In other examples, expression of a particular DNA or RNA can be detected in about 1 to 1000 cells (such as about 1 to 500 cells, about 1 to 250 cells, about 1 to 100 cells, about 1 to 50 cells, about 1 to 25 cells, or about 1 cell).

Samples may be treated prior to (or contemporaneous with) contacting the sample with a buffer, e.g., lysis buffer, which conserves all components of the sample in a single solution. In one example, modified nucleic acids (such as, DNA or RNA) are captured and purified from cells, for example using the methods provided herein to separate those nucleic acid molecules away from other biological components in a sample and unmodified nucleic acid molecules. Purification refers to separating the modified nucleic acids from one or more extraneous components also found in a sample. Components that are isolated, extracted or purified (such as modified nucleic acids) from a mixed specimen or sample (e.g., one also containing unmodified nucleic acids) typically are enriched by at least 50%, at least 60%, at least 75%, at least 90%, or at least 98% or even at least 99% compared to the unpurified or non-extracted sample.

In some examples, cells in the sample are lysed or permeabilized in an aqueous solution (for example using a lysis buffer). The aqueous solution or lysis buffer includes detergent (such as sodium dodecyl sulfate, SDS) and one or more chaotropic agents (such as formamide, guanidinium HCl, guanidinium isothiocyanate, or urea). The solution may also contain a buffer (for example SSC). In some examples, the lysis buffer includes about 15% to 25% formamide (v/v) about 0.01% to 0.1% SDS, and about 0.5-6×SSC (for example, about 3×SSC). The buffer may optionally include tRNA (for example, about 0.001 to about 2.0 mg/ml) or a ribonuclease; DNAase; proteinase K; enzymes (e.g., collagenase or lipase) that degrade protein, matrix, carbohydrate, lipids, or one species of oligonucleotides, or combinations thereof. The lysis buffer may also include a pH indicator, such as Phenol Red. In a particular example, the lysis buffer includes 20% formamide, 3×SSC (79.5%), 0.05% SDS, 1 μg/ml tRNA, and 1 mg/ml Phenol Red. Cells are incubated in the aqueous solution or lysis buffer (optionally overlayed with oil to prevent evaporation or to serve as a sink for paraffin) for a sufficient period of time (such as about 1 minute to about 60 minutes, for example about 5 minutes to about 20 minutes, or about 10 minutes) and at a sufficient temperature (such as about 22° C. to about 110° C., for example, about 80° C. to about 105° C., about 37° C. to about 105° C., or about 90° C. to about 100° C.) to lyse or permeabilize the cell. In some examples, lysis is performed at about 95° C. In one example Proteinase K is included with the lysis buffer. In one example DNAase is included with the lysis buffer.

Nucleic acids (such as modified nucleic acids) can be isolated from the cell lysate prior to contact with one or more NPPFs, for example using the methods provided herein (e.g., see example in FIG. 9).

In other examples, tissue samples are prepared by fixing and embedding the tissue in a medium or include a cell suspension is prepared as a monolayer on a solid support (such as a glass slide), for example by smearing or centrifuging cells onto the solid support. In further examples, fresh frozen (for example, unfixed) tissue or tissue sections may be used in the methods disclosed herein. In particular examples, FFPE tissue sections are used as a source of nucleic acids to be sequenced in the disclosed methods.

In some examples an embedding medium is used. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics. Many embedding media are hydrophobic; therefore, the inert material may need to be removed prior to analysis, which utilizes primarily hydrophilic reagents. The term deparaffinization or dewaxing is broadly used herein to refer to the partial or complete removal of any type of embedding medium from a biological sample. For example, paraffin-embedded tissue sections are dewaxed by passage through organic solvents, such as toluene, xylene, limonene, or other suitable solvents. In other examples, paraffin-embedded tissue sections are utilized directly (e.g., without a dewaxing step).

Tissues can be fixed by any suitable process, including perfusion or by submersion in a fixative. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum.

The most commonly used fixative in preparing tissue or cell samples is formaldehyde, generally in the form of a formalin solution (4% formaldehyde in a buffer solution, referred to as 10% buffered formalin). In one example, the fixative is 10% neutral buffered formalin, and thus in some examples the sample is formalin fixed.

In some examples, the sample is an environmental sample (such as a soil, air, or water sample, or a sample obtained from a surface (for example by swabbing)), or a food sample (such as a vegetable, fruit, dairy or meat containing sample) for example containing pathogen nucleic acid molecules. Thus, modified nucleic acids can be captured from such samples.

### VII. Modified Nucleic Acid Molecules

Modified nucleic acid molecules whose sequence can be determined using the disclosed methods include those having one or more base or sugar modifications, for example as a result of exposure to an agent, such as a therapeutic agent, mutagen, or stress. In one example, a modified nucleic acid includes one or more m⁶A bases. Modified nucleic acid molecules include single-, double- or other multiple-stranded nucleic acid molecules (such as, DNA (e.g., genomic, mitochondrial, or synthetic), RNA (such as mRNA, miRNA, tRNA, siRNA, long non-coding (nc) RNA, biologically occurring anti-sense RNA, Piwi-interacting RNAs (piRNAs), or small nucleolar RNAs (snoRNAs)), whether from eukaryotes, prokaryotes, viruses, fungi, bacteria or other biological organism. Genomic modified nucleic acids may include one or several parts of the genome, such as coding regions (e.g., genes or exons), non-coding regions (whether having known or unknown biological function, e.g., enhancers, promoters, regulatory regions, telomeres, or “nonsense” DNA). In some embodiments, a modified nucleic acid molecule may contain naturally occurring genetic (e.g., germ line or somatic) mutations. Such mutations may include (or result from) genomic rearrangements (such as translocations, insertions, deletions, or inversions), single nucleotide variations, and/or genomic amplifications. In some embodiments, a modified nucleic acid molecule may contain one or more modified or synthetic monomers units (e.g., peptide nucleic acid (PNA), locked nucleic acid (LNA), methylated nucleic acid, post-translationally modified amino acid, cross-linked nucleic acid or cross-linked amino acid.

The portion of a modified nucleic acid molecule to which a NPPF may specifically bind may be referred to as “target,” again, as context dictates, but more specifically may be referred to as target portion, complementary region (CR), target site, protected target region or protected site, or similar. A NPPF specifically bound to its complementary region forms a complex, which complex may remain integrated with the modified nucleic acid molecule as a whole or be separate (or be or become separated) from the modified nucleic acid molecule as a whole. In some embodiments, a NPPF/CR complex is separated (or becomes disassociated) from the modified nucleic acid molecule as a whole and/or the reaction, e.g., by the action of a nuclease, such as S1 nuclease.

All types of modified nucleic acid molecules can be analyzed using the disclosed methods. In one example, the modified nucleic acid molecule is a modified ribonucleic acid (RNA) molecule, such as a modified messenger RNA (mRNA), a modified ribosomal RNA (rRNA), a modified transfer RNA (tRNA), modified micro RNA (miRNA), a modified siRNA, anti-sense RNA, modified long non coding RNA (lincRNA or lncRNA), modified circular RNA (circRNA), or a modified viral RNA (vRNA). In another example, the modified nucleic acid molecule is a modified deoxyribonucleic (DNA) molecule, such as genomic modified DNA (gDNA), modified mitochondrial DNA (mtDNA), modified chloroplast DNA (cpDNA), modified viral DNA (vDNA), cDNA, or a modified transfected DNA. In some examples, the whole transcriptome or epitranscriptome of a cell or a tissue can be analyzed using the disclosed methods. In one example, the modified nucleic acid molecule is a rare modified nucleic acid molecule, for example only appearing less than about 100,000 times, less than about 10,000 times, less than about 5,000 times, less than about 100 times, less than 10 times, or only once in the sample, such as a nucleic acid molecule only appearing 1 to 10,000, 1 to 5,000, 1 to 100 or 1 to 10 times in the sample.

A plurality of modified nucleic acid molecules can be detected or sequenced in the same sample or assay, or even in multiple samples or assays, for example simultaneously or contemporaneously. Similarly, a single modified nucleic acid molecule can be detected or sequenced in a plurality of samples, for example simultaneously or contemporaneously. In one example the modified nucleic acid molecules are a modified miRNA and a modified mRNA. Thus, in such an example, the method would include the use of at least one NPPF specific for the modified miRNA and at least one NPPF specific for the modified mRNA. In one example the modified nucleic acid molecules are two different modified DNA molecules. Thus, in such an example, the method would include the use of at least one NPPF specific for the first modified DNA and at least one NPPF specific for the second modified DNA. In one example the modified nucleic acid molecules are two different modified RNA molecules. Thus, in such an example, the method would include the use at least one NPPF specific for the first modified RNA and at least one NPPF specific for the second modified RNA.

In some examples, the disclosed methods permit direct or indirect sequencing of modified DNA or modified RNA containing single nucleotide polymorphisms (SNPs) or variants (sNPVs), splice junctions, methylated DNA, gene fusions or other mutations, protein-bound DNA or RNA, and also cDNA, as well as levels of expression (such as DNA or RNA expression, such as cDNA expression, mRNA expression, miRNA expression, rRNA expression, siRNA expression, or tRNA expression). Any modified nucleic acid molecule to which a nuclease protection probe can be designed to hybridize can be quantified and identified by the disclosed methods, even though the modified nucleic acid molecules themselves need not be sequenced and are even in some examples destroyed (e.g., the NPPF hybridized to the modified nucleic acid molecule is sequenced as a surrogate for the modified nucleic acid molecule).

One skilled in the art will appreciate that the modified nucleic acid molecule can include a combination of natural and unnatural bases.

In specific non-limiting examples, a modified nucleic acid (such as a modified DNA or modified RNA) is associated with a neoplasm (for example, a cancer). Numerous chromosome abnormalities (including translocations and other rearrangements, reduplication or deletion) or mutations have been identified in neoplastic cells, especially in cancer cells, such as B-cell and T-cell leukemias, lymphomas, breast cancer, colon cancer, neurological cancers and the like.

In some examples, a modified nucleic acid molecule includes GAPDH (e.g., GenBank Accession No. NM_002046), PPIA (e.g., GenBank Accession No. NM_021130), RPLP0 (e.g., GenBank Accession Nos. NM_001002 or NM_053275), RPL19 (e.g., GenBank Accession No. NM_000981), ZEB1 (e.g., GenBank Accession No. NM_030751), Zeb2 (e.g., GenBank Accession Nos. NM_001171653 or NM_014795), CDH1 (e.g., GenBank Accession No. NM_004360), CDH2 (e.g., GenBank Accession No. NM_007664), VIM (e.g., GenBank Accession No. NM_003380), ACTA2 (e.g., GenBank Accession No. NM_001141945 or NM_001613), CTNNB1 (e.g., GenBank Accession No. NM_001904, NM 001098209, or NM_001098210), KRT8 (e.g., GenBank Accession No. NM_002273), SNAI1 (e.g., GenBank Accession No. NM_005985), SNAI2 (e.g., GenBank Accession No. NM_003068), TWIST1 (e.g., GenBank Accession No. NM_000474), CD44 (e.g., GenBank Accession No. NM_000610, NM_001001389, NM_00100390, NM_001202555, NM_001001391, NM_001202556, NM_001001392, NM_001202557), CD24 (e.g., GenBank Accession No. NM_013230), FN1 (e.g., GenBank Accession No. NM_212474, NM_212476, NM 212478, NM 002026, NM_212482, NM 054034), IL6 (e.g., GenBank Accession No. NM_000600), MYC (e.g., GenBank Accession No. NM_002467), VEGFA (e.g., GenBank Accession No. NM_001025366, NM_001171623, NM_003376, NM_001171624, NM_001204384, NM_001204385, NM_001025367, NM_001171625, NM_001025368, NM_001171626, NM_001033756, NM_001171627, NM_001025370, NM_001171628, NM_001171622, NM_001171630), HIF1A (e.g., GenBank Accession No. NM_001530, NM_181054), EPAS1 (e.g., GenBank Accession No. NM_001430), ESR2 (e.g., GenBank Accession No. NM_001040276, NM_001040275, NM_001214902, NM_001437, NM_001214903), PRKCE (e.g., GenBank Accession No. NM_005400), EZH2 (e.g., GenBank Accession No. NM_001203248, NM_152998, NM_001203247, NM_004456, NM_001203249), DAB2IP (e.g., GenBank Accession No. NM_032552, NM_138709), B2M (e.g., GenBank Accession No. NM_004048), and SDHA (e.g., GenBank Accession No. NM_004168).

In some examples, a modified miRNA includes hsa-miR-205 (MIR205, e.g., GenBank Accession No. NR_029622), hsa-miR-324 (MIR324, e.g., GenBank Accession No.NR_029896), hsa-miR-301a (MIR301A, e.g., GenBank Accession No. NR_029842), hsa-miR-106b (MIR106B, e.g., GenBank Accession No. NR_029831), hsa-miR-877 (MIR877, e.g., GenBank Accession No. NR_030615), hsa-miR-339 (MIR339, e.g., GenBank Accession No. NR_029898), hsa-miR-10b (MIR10B, e.g., GenBank Accession No. NR_029609), hsa-miR-185 (MIR185, e.g., GenBank Accession No. NR_029706), hsa-miR-27b (MIR27B, e.g., GenBank Accession No. NR_029665), hsa-miR-492 (MIR492, e.g., GenBank Accession No. NR_030171), hsa-miR-146a (MIR146A, e.g., GenBank Accession No. NR_029701), hsa-miR-200a (MIR200A, e.g., GenBank Accession No. NR_029834), hsa-miR-30c (e.g., GenBank Accession No. NR_029833, NR_029598), hsa-miR-29c (MIR29C, e.g., GenBank Accession No. NR_029832), hsa-miR-191 (MIR191, e.g., GenBank Accession No. NR_029690), or hsa-miR-655 (MIR655, e.g., GenBank Accession No. NR_030391).

In one example the modified nucleic acid is a modified pathogen nucleic acid molecule, such as modified viral RNA or viral DNA. Exemplary pathogens include, but are not limited to, viruses, bacteria, fungi, and protozoa. In one example, the modified nucleic acid is a modified viral RNA. Viruses include positive-strand RNA viruses and negative-strand RNA viruses. Exemplary positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); and Coronaviruses (examples of which include SARS coronaviruses, such as the Urbani strain). Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses). In one example the modified nucleic acid molecule is modified viral DNA from a DNA virus, such as Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19). In another example, the modified nucleic acid is a modified retroviral nucleic acid, such as one from human immunodeficiency virus type 1 (HIV-1), such as subtype C, HIV-2; equine infectious anemia virus; feline immunodeficiency virus (FIV); feline leukemia viruses (FeLV); simian immunodeficiency virus (SIV); and avian sarcoma virus. In one example, the modified nucleic acid is a modified bacterial nucleic acid. In one example the modified bacterial nucleic acid is from a gram-negative bacteria, such as Escherichia coli (K-12 and O157:H7), Shigella dysenteriae, and Vibrio cholerae. In another example the modified bacterial nucleic acid is from a gram-positive bacteria, such as Bacillus anthracis, Staphylococcus aureus, pneumococcus, gonococcus, and streptococcal meningitis. In one example, the modified nucleic acid is a nucleic acid from protozoa, nemotodes, or fungi. Exemplary protozoa include, but are not limited to, Plasmodium, Leishmania, Acantharnoeba, Giardia, Entamoeba, Cryptosporidium, Isospora, Balantidium, Trichomonas, Trypanosoma, Naegleria, and Toxoplasma. Exemplary fungi include, but are not limited to, Coccidiodes immitis and Blastomyces dermatitidis. One skilled in the art will appreciate that any of these microbes listed can be used as a test agent on a cell or organism (e.g., infect the cell or organism), and the effect of such a microbe on modifying the nucleic acids of the cell or organism determined using the disclosed methods.

One of skill in the art can identify additional modified DNAs or modified RNAs and/or additional modified miRNAs which can be sequenced utilizing the methods disclosed herein.

### VIII. Assay Output

In some embodiments, the disclosed methods include determining the presence or an amount of one or more modified nucleic acid molecules in a sample, for example by determining the sequence of one or more modified nucleic acid molecules in a sample. Thus, in some examples, the amount of particular modified nucleic acid molecules in a sample is quantified. The results of the methods can be provided to a user (such as a scientist, clinician or other health care worker, laboratory personnel, or patient) in a perceivable output that provides information about the results of the test. In some examples, the output can be a paper output (for example, a written or printed output), a display on a screen, a graphical output (for example, a graph, chart, or other diagram), or an audible output. In one example, the output is a table or graph including a qualitative or quantitative indicator of presence or amount (such as a normalized amount) of a modified nucleic acid molecule detected (or not detected) in the sample. In other examples the output is a map or image of signal present on a substrate (for example, a digital image of fluorescence from an array). In other examples, the embodiments, the output is the sequence of one or more modified nucleic acid molecules in a sample, such a report indicting the presence of a particular mutation in the modified molecule, such as a report indicting whether a test agent produced target and/or off-target modifications of the treated genome.

In some examples, the output is a numerical value, such as an amount of a modified nucleic acid molecule in a sample, or the percentage of modified nucleic acid present in the pool of all copies of that nucleic acid within the sample. In additional examples, the output is a graphical representation, for example, a graph that indicates the value (such as amount or relative amount) of a modified nucleic acid molecule in the sample on a standard curve. In additional examples, the output is a graphical representation, for example, a graph that indicates the sequence of a modified nucleic acid molecule in the sample (for example which might indicate where a modification is present and/or whether the modification is on or off target). In some examples, the output is communicated to the user, for example by providing an output via physical, audible, or electronic means (for example by mail, telephone, facsimile transmission, email, or communication to an electronic medical record).

The output can provide quantitative information (for example, an amount of a particular modified nucleic acid molecule or an amount of a particular modified nucleic acid molecule relative to a control sample or value) or can provide qualitative information (for example, a determination of presence or absence of a particular modified nucleic acid molecule). In additional examples, the output can provide qualitative information regarding the relative amount of a modified nucleic acid molecule in the sample, such as identifying an increase or decrease relative to a control or no change relative to a control.

As discussed herein the NPPF amplicons can include one or more experiment tags, which can be used for example to identify a particular patient, sample, experiment, or modified nucleic acid molecule. The use of such tags permits the detected or sequenced NPPF amplicon to be “sorted” or even counted, and thus permits analysis of multiple different samples (for example from different patients), multiple different modified nucleic acid molecules (for example at least two different modified nucleic acid molecules), or combinations thereof, in a single reaction. In one example, Illumina and Bowtie software can be used for such analysis.

In one example, the NPPFs include an experiment tag unique for each different modified nucleic acid molecule. The use of such a tag allows one to merely sequence or detect this tag, without sequencing the entire NPPF, to identify the NPPF as corresponding to a particular modified nucleic acid molecule. In addition, if multiple modified nucleic acid molecules are to be analyzed, the use of a unique experiment tag for each modified nucleic acid molecule simplifies the analysis, as each detected or sequenced experiment tag can be sorted, and if desired counted. This permits for example quantification of the modified nucleic acid molecule that was in the sample, as the NPPF amplicons are in stoichiometric proportion to the modified nucleic acid molecule in the sample. For example, if multiple modified nucleic acids are detected or sequenced in a sample, the methods permit the generation of a table or graph showing each modified nucleic acid molecule and the number of copies detected or sequenced, by simply detecting or sequencing and then sorting the experimental tag.

In another example, the NPPFs include an experiment tag unique for each different sample (such as a unique tag for each patient sample). The use of such a tag allows one to associate a particular detected NPPF amplicon with a particular sample. Thus, if multiple samples are analyzed in the same reaction (such as the same well or same sequencing reaction), the use of a unique experiment tag for each sample simplifies the analysis, as each detected or sequenced NPPF can be associated with a particular sample. For example, if a modified nucleic acid is detected or sequenced in samples, the methods permit the generation of a table or graph showing the result of the analysis for each sample.

One skilled in the art will appreciate that each NPPF amplicon can include a plurality of experiment tags (such as at least 2, 3, 4, 5, 6, 7, 8, 9 or 10 experiment tags), such as a tag representing the modified nucleic acid molecule, and another representing the sample. Once each tag is detected or sequenced, appropriate software can be used to sort the data in any desired format, such as a graph or table. For example, this permits analysis of multiple modified nucleic acid molecules in multiple samples simultaneously or contemporaneously.

In some examples, the detected or sequenced NPPF amplicon is compared to a reference database of known sequences for each modified nucleic acid molecule. In some examples, such a comparison permits detection of mutations, such as SNPs. In some examples, such a comparison permits for a comparison of a reference NPPFs abundance to the abundance of an NPPF probe in a region known to contain SNPs.

The disclosure is further illustrated by the following non-limiting Examples.

### Example 1

Enrichment for and Detection of Modified RNAs within a Cell Lysate Sample.

This example describes methods used to enrich for and isolate modified RNAs within a sample of modified and unmodified RNAs, followed by detection and measurement of the levels of the modified and unmodified RNAs using a nuclease-protection mediated sequencing assay provided herein (HTG EdgeSeq). The RNA modification in this example is N⁶-methyladenosine (m⁶A). One skilled in the art will appreciate that similar methods can be used for other RNA modifications.

The sample used was an SuDHL6 cell line lysate in a lysis buffer. To provide a set of external controls for the enrichment reaction, four synthetic RNAs were generated. These four RNAs were generated using in vitro transcription (IVT) and are referred to herein as IVTs. Two of these IVTs were generated using a 1:5 ratio of N⁶-methyladenosine-5′-triphosphate (m⁶ATP):rATP in the transcription reaction, to generate modified RNAs. The other two are unmodified controls and were transcribed without any modified nucleotides. The relative abundance of these four IVTs can be measured by sequencing, such as nuclease-protection mediated sequencing, such as an HTG EdgeSeq assay (see U.S. Pat. No. 8,741,564, herein incorporated by reference in its entirety). HTG EdgeSeq assays are targeted, quantitative RNA sequencing assays that use nuclease protection to measure RNA abundance. A set of PCR primers for RT-qPCR (Table 2) were designed to provide a comparison method for measuring the IVTs.

TABLE 2
Primer and sequences used.

| Primer name | Sequence (5′-3′) (SEQ ID NO:) |
|---|---|
| ER-0099 FWD | TTGCCCGATGCAATGAGA (1) |
| ER-0099 REV | CGCCTGAAGAAGCTGAGATAA (2) |
| ER-0109 FWD | CATGATTGTGACCTCGGTATCTC (3) |
| ER-0109 REV | GGTTCAAGGGTGAGGCTATTT (4) |

For this example, an equimolar mixture of the four IVTs was generated. The sample comprised 0.1 fmol of each IVT in a cell lysate solution. To demonstrate the ability to enrich for the modified IVTs within this lysate milieu, the sample was used in an immunoprecipitation reaction followed by detection. The immunoprecipitation was performed in triplicate. Each reaction comprised:
0.1 fmol of each IVT,
80,000 SuDHL6 cells in a lysis buffer,
1 μl RNasin to prevent RNase activity,
20 μl of protein G beads (Thermo) pre-conjugated with an anti-N⁶-methyladenosine antibody from New England Biolabs

Immunoprecipitation was performed for 2 hours at room temperature, with mixing. Immunoprecipitation was followed by washing of the beads using three wash buffers (Buffer 1: 150 mM NaCl, 0.1% IGEPAL CA-630, 10 mM Tris pH 7.4; Buffer 2: 50 mM NaCl, 0.1% IGEPAL CA-630, 10 mM Tris pH 7.4; Buffer 3: 500 mM NaCl, 0.1% IGEPAL CA-630, 10 mM Tris pH 7.4). To track the enrichment reaction, aliquots of the reaction were removed after the immunoprecipitation and after the first wash. RNAs were isolated from all aliquots (the immunoprecipitation, wash, and final washed beads) using a standard method, and the resulting RNA samples were then assessed using two methods (RT-qPCR and HTG EdgeSeq).

In the RT-qPCR detection method, the RNA samples were reverse-transcribed and aliquots of the reverse-transcription reaction were amplified in a qPCR reaction using the primers described above. The IVT mixture itself was used as a positive control. qPCR reactions were performed in triplicate for each sample and set of primers.

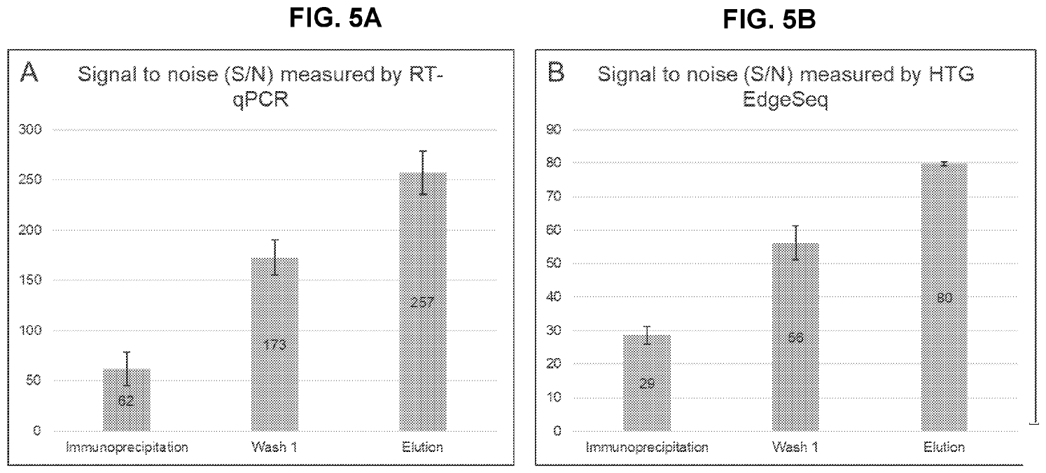

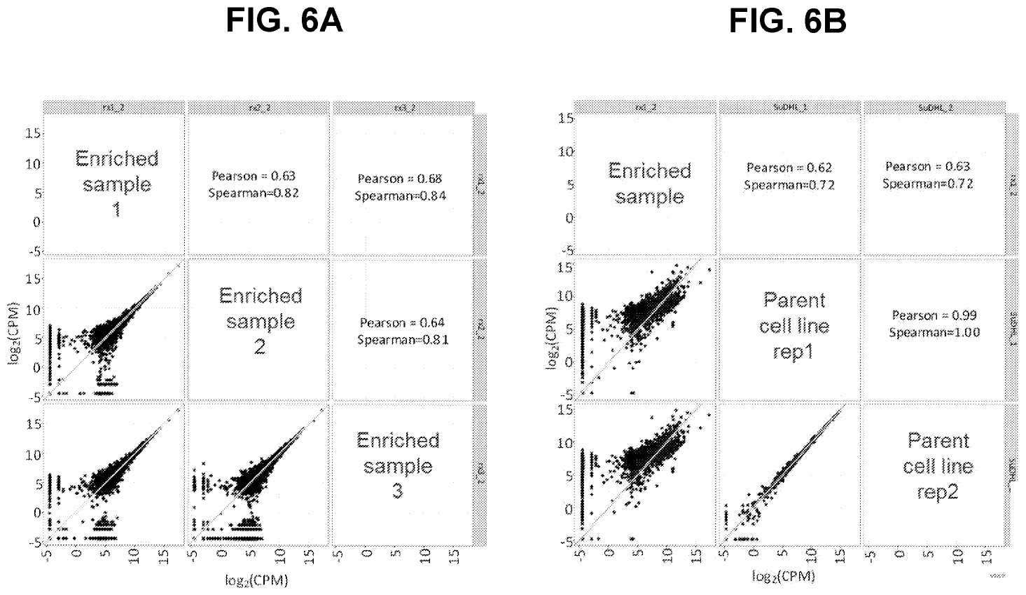

The Ct values for each sample were adjusted relative to the control, and a “signal-to-noise” ratio (S/N) or specificity score was calculated. The “signal” was the amount of the modified IVT in the enriched sample, and the “noise” the amount of unmodified IVT in the enriched sample. The triplicate reactions were averaged and graphed in FIG. 5A; the error bars on the graph are one standard deviation from the mean.

In the HTG EdgeSeq detection method, the RNA samples were assessed using the HTG EdgeSeq Oncology Biomarker Panel, a targeted RNAseq assay. This assay contains NPPFs to measure the IVTs in the sample. It also contains NPPFs for approximately 2500 other RNAs, allowing the assessment of cellular modified RNAs enriched by the described process. For the external control IVTs, a signal-to-noise ratio (S/N) was calculated from the raw data from the assay, normalized to the signal-to-noise ratio of the external spike ins in a non-enriched sample. The triplicate reactions were averaged and graphed in FIG. 5B; the error bars on the graph are one standard deviation from the mean.

FIG. 6A and FIG. 6B shows two aspects of the results from enrichment of the cellular RNAs. The first is the correlation between replicate enrichment reactions, and the second is the correlation between enrichment reactions and the parent, non-enriched sample.

The results demonstrate four points.

First, the measurement of signal-to-noise, which represents the success of the enrichment reaction itself, is both high and highly repeatable using both detection methods, demonstrating that the disclosed enrichment method is itself repeatable (FIGS. 5A and 5B; see “Elution” for the final product sample of the enrichment reaction). The absolute value returned by the two detection methods differs, but the trend is the same.

Second, replicate enrichment reactions generate repeatable data when observing both the external spike in IVTs and the enriched internal modified RNAs from the cell line itself. These results are only observed using the HTG EdgeSeq detection method. FIG. 6A and FIG. 6B shows a scatterplot correlation of three separate enrichment reactions. The Spearman correlations range from 0.81 to 0.86. Although there is some variability at the lower end of the expression range, the correlation is strong.

Third, washing is effective in reducing “noise” from the system. As the washes progress, a greater S/N ratio is observed, indicating that washing can increase S/N (FIG. 5A and FIG. 5B).

Finally, enrichment alters the resulting expression profile when compared to the parent cell line sample. The scatterplot FIG. 6B shows HTG EdgeSeq results for two replicates of the parent cell line and a single replicate of the enriched sample. The scatterplot shows that the replicate parent cell line results are very highly correlated. However, there is a clear deviation from the correlation between the enriched and non-enriched sample. The deviation is both in the positive (enrichment of modified RNAs) and negative (loss of unmodified RNAs) directions; as is desired for an enrichment technique.

These results demonstrate the ability of the disclosed methods to enrich for modified RNAs within a sample and assess their abundance using HTG EdgeSeq. The results for the external controls are corroborated by RT-qPCR, and those external controls can be used to track and demonstrate the level of enrichment and the efficacy of the wash conditions.

### Example 2

Enrichment and Detection of Modified RNAs within a DNase-Treated Biological Sample with External Controls.

This example describes methods used to enrich for and isolate modified RNAs within a cell lysate sample, followed by detection using a nuclease-protection mediated sequencing assay. DNA is first depleted from the sample to avoid possible false-positive signals from modified DNA.

The antibody used for enrichment in Examples 1 and 2 recognizes N⁶-methyadenoside residues of both DNA and RNA. In most applications using this antibody (or this class of antibodies), the sample used for immunoprecipitation is either (only) RNA or (only) DNA. However, the methods described herein can be used with crude cell lysates, which will contain both RNA and DNA. Further, DNA may be measured by the detection assay, such as a nuclease-protection mediated sequencing assay, such as an HTG EdgeSeq assay. Therefore, if modified DNA species are enriched for and detected by the described methods, they could generate false-positive signals. In this Example, it is first shown that modified DNA is a product of the immunoprecipitation or enrichment reaction and that such DNA can indeed be measured by the HTG EdgeSeq reactions. This Example then shows the effect of addition of a DNase treatment of the sample, either prior to enrichment by immunoprecipitation, or following it, and the effects on the resulting data.

To demonstrate that DNA can be detected by the HTG EdgeSeq assay, the following experiment was performed. A cell lysate generated from SuDHL6 cells was used as the sample. One set of lysates were treated with DNase and another set was not; approximately 80,000 cells were used per sample and both treated and untreated cells were run in triplicate. These lysates were assessed using the HTG EdgeSeq Oncology Biomarker Panel to which a set of NPPFs designed to detect DNA from various chromosomes was added. A log₂ counts per million (log₂CPM) standardization was applied to the data from each sample, and an average of the triplicate reactions for each sample was calculated. Table 3 shows the resulting data from DNase-treated (column 2) and non-treated (columnm 3) samples. Only the data from the DNA probes themselves are displayed in the table. These data demonstrate that the 1110 EdgeSeq assay measures DNA, and DNase treatment greatly depletes the DNA from the sample to a negligible level.

TABLE 3
Data from DNase-treated and non-treated samples.

| | SuDHL6 + DNase | SuDHL6 − DNase |
|---|---|---|
| gDNA_Ch_02-T1 | −4.743 | 5.972 |
| gDNA_Ch_02-T2 | −4.215 | 7.693 |
| gDNA_Ch_02-T3 | 0.946 | 8.040 |
| gDNA_Ch_02-T5 | 3.318 | 8.354 |
| gDNA_Ch_04-T2 | −1.041 | 7.275 |
| gDNA_Ch_04-T3 | −0.309 | 8.759 |
| gDNA_Ch_05-T2 | −3.033 | 7.662 |
| gDNA_Ch_06-T1 | −0.776 | 8.628 |
| gDNA_Ch_06-T4 | 0.640 | 7.789 |
| gDNA_Ch_07-T1 | −0.427 | 8.836 |
| gDNA_Ch_07-T3 | 0.045 | 8.165 |
| gDNA_Ch_09-T3 | −2.630 | 8.966 |
| gDNA_Ch_09-T4 | 1.024 | 8.017 |
| gDNA_Ch_11-T1 | −0.572 | 7.810 |
| gDNA_Ch_12-T2 | 0.267 | 7.697 |
| gDNA_Ch_12-T4 | 0.443 | 7.815 |
| gDNA_Ch_12-T5 | −0.727 | 8.243 |
| gDNA_Ch_14-T2 | 3.221 | 8.634 |
| gDNA_Ch_19-T4 | 2.788 | 8.094 |
| gDNA_Ch_19-T6 | −2.667 | 7.877 |
| gDNA_Ch_22-T1 | 1.372 | 7.279 |
| gDNA_Ch_23-T2 | −0.876 | 7.497 |

To demonstrate that modified DNA is enriched by the immunoprecipitation reaction, a second experiment was performed in which enrichment was performed on a SuDHL6 lysate sample, followed by treatment of the enriched sample with DNase. A control sample was not treated with DNase, and both treated and untreated enriched samples were assessed via HTG EdgeSeq. The SuDHL6 cell lysate was spiked with IVTs (as described in Example 1). The immunoprecipitation reaction comprised:
120,000 SuDHL cells in a lysis buffer,
50 amol of each external spike in IVT,
1 μl RNasin to prevent RNase activity,
an immunoprecipitation buffer,
20 μl of protein G beads (Thermo) pre-conjugated with an anti-N⁶-methyladenosine antibody from New England Biolabs

Immunoprecipitation and washing of the beads was performed as described in Example 1. RNAs were eluted from the beads via treatment with Proteinase K, to digest the antibody away from the beads. This elution was followed by either treatment with DNase, or no treatment. The resulting samples were then assessed using an HTG EdgeSeq assay.

The HTG EdgeSeq assay is a sequencing-based assay in which each sample forms a sub-library of the final sequencing library. As part of the generation of the final library, each sample's sub-library is quantitated using a qPCR assay. Quantitation of the sub-library from the non-treated sample showed a concentration of 10.9 nM, whereas the DNase-treated sub-library had a concentration of 6.6 nM. This indicated that the DNase treatment removed a substantial amount of the assessable material prior to assessment via HTG EdgeSeq. However, the sequencing results showed that the IVT spike in controls, used to test the enrichment reaction, were unaffected, as the signal-to-noise ratio for the two samples was similar (57× and 55×). These results demonstrate that that modified DNA is recognized by the antibody used for the immunoprecipitation reaction, and that DNA is measurable within the HTG EdgeSeq assay. But DNAse treatment, of either the initial cell line lysate or the enriched sample following immunoprecipitation, removes the majority of this unwanted DNA.

A final enrichment experiment was performed in which a HEK293T cell line lysate was either treated with DNase or not treated, the enrichment was performed, and the enriched sample assessed using HTG EdgeSeq. The lysate was spiked with IVTs with and without modifications (as described in Example 1). The immunoprecipitation reaction comprised:
55,000 HEK293T cells in a lysis buffer,
10 amol of each IVT within the lysate,
1 μl RNasin to prevent RNase activity,
an immunoprecipitation buffer,
20 μl of protein G beads (Thermo) pre-conjugated with N⁶-methyladenosine antibody from New England Biolabs

Immunoprecipitation and washing of the beads was performed as described in Example 1. RNAs were eluted from the beads via addition of a lysis buffer followed by heating to 95° C. for 15 minutes. The resulting enriched RNA samples were then assessed using an HTG EdgeSeq assay that contained NPPFs designed to measure genomic DNA. As a control, the DNase-treated parent sample, without enrichment, was also assessed using the same HTG EdgeSeq assay.

To determine whether the DNase treatment was effective, the signal measured by the genomic DNA probes within the assay was compared to the signal measured by the negative control probes within the assay. The signal intensity for these two probe sets were both low and very comparable as seen in Table 4. For comparison, the average positive control signal was three orders of magnitude larger than the average signal from the negative or genomic probe sets. These data indicate that there is little to no DNA present in these samples (such as less than 10%, such as less than 1% of the total DNA survived the treatment), and therefore the DNase treatment has been effective.

TABLE 4
Average signal intensity for different probe sets.

| | Rx1 | Rx2 | Rx3 |
|---|---|---|---|
| Average signal, genomic DNA probes | 30.7 | 23.3 | 34.1 |
| Average signal, negative control probes | 24.7 | 31.0 | 30.5 |
| Average signal, positive control probes | 33026.5 | 28913.25 | 30379 |

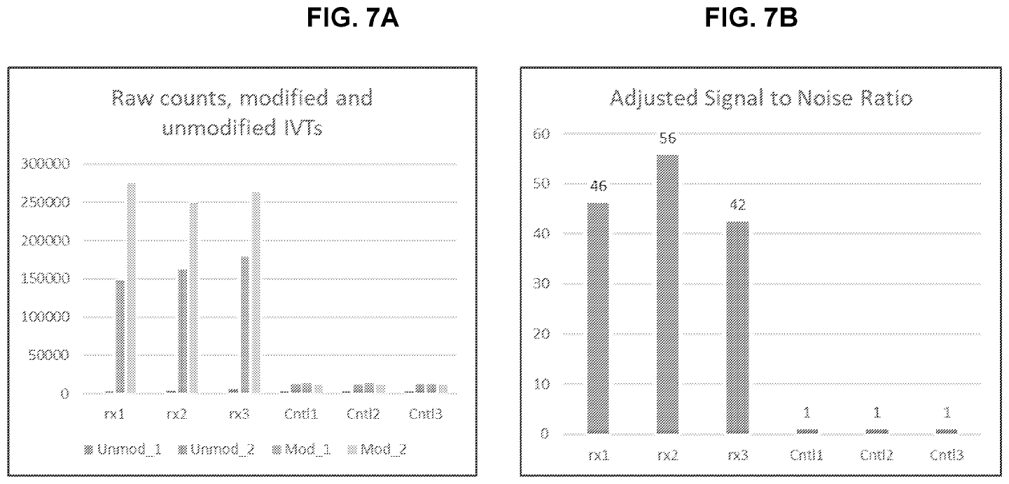

The enriched, DNase-treated samples were assessed for the efficacy of enrichment. The signal-to-noise ratio was calculated using the raw signal from the modified and unmodified IVT external spike in controls (the modified IVT external spike is labeled as “Mod_1” and “Mod_2” while the unmodified IVT external spike is labeled “Unmod_1” and “Unmod_2”). The raw signal for these IVTs for both enriched samples (denoted as “rx1”, “rx2”, and “rx3” along the y-axis of the graph in FIG. 7A) and non-enriched controls (denoted as “Cntl1”, “Cntl2”, and “Cntl3” along the y-axis of the graph in FIG. 7A) are graphed in FIG. 7A. The raw counts of Unmod_1, Unmod_2, Mod_1, and Mod_2 are shown as a set of four bars for each of rx1, rx2, rx3, Cntl1, Cntl2, and Cntl3, with the left most bar of each set representing the raw count of Unmod_1, the second bar from the left of each set showing the raw count of Unmod_2, the third bar from the left of each set showing the raw count of Mod_1, and the right most bar of each set representing the raw count of Mod_2. Note that the raw counts of Unmod_1 and Unmod_2 for each of rx1, rx2, and rx3 were so low that the graph does not illustrate a bar for Unmod_1 for those three reactions and the bar for Unmod_2 is just barely visible.

A signal-to-noise ratio was calculated by averaging the signal for the unmodified IVTs (“noise”) and the signal for the modified IVTs (“signal”) and taking the ratio. The ratio was adjusted for the enriched samples to account for the ratio in the non-enriched controls. The adjusted results for each enriched sample and the controls are shown in FIG. 7B. The signal-to-noise ratios for the enriched samples were 42 or greater.

The data presented in FIGS. 7A and 7B demonstrate the efficacy of the enrichment by viewing the external controls. However, cellular transcripts within the sample may also demonstrate evidence of enrichment; albeit in a more heterogeneous manner—i.e., for any given RNA within the cell, a fraction of the transcript pool for that RNA may have an m⁶A modification. Therefore, there is not an expectation that every modified RNA will demonstrate the same enrichment as the controls, which are a homogeneous pool and are likely behave in a more binary fashion. Therefore, enrichment of modified cellular RNAs was examined in two ways.

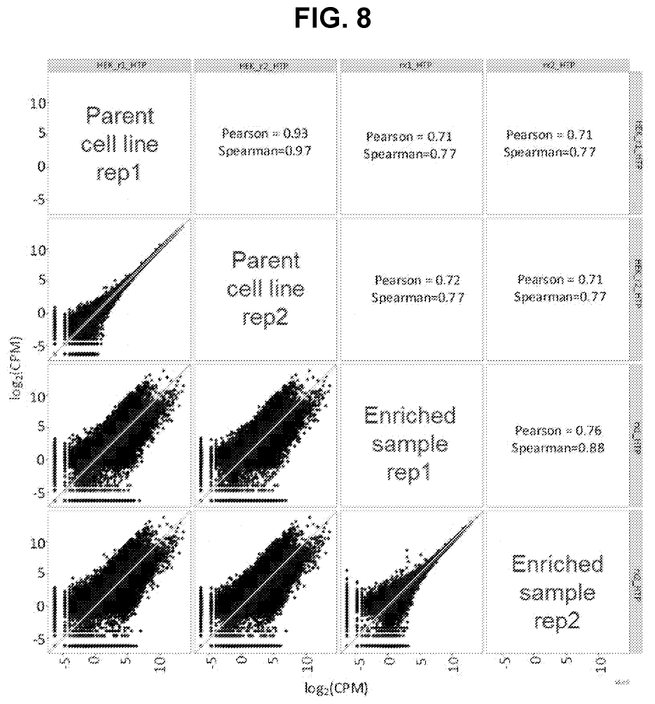

First, a scatterplot correlation between enriched and non-enriched parent samples was plotted, in FIG. 8. This plot shows excellent correlation between two replicates of the non-enriched parent samples (Spearman correlation coefficient of 0.97), good correlation between two replicates of enrichment samples (Spearman correlation coefficient of 0.88), and poorer correlation between the two types of sample (Spearman correlation coefficient of 0.77). The spread of signal away from the perfect unity line for the comparison between enriched and non-enriched samples is in both directions, as would be expected for an enrichment reaction in which some RNAs are enriched for and others depleted.

These results demonstrate the ability of the disclosed methods to enrich for modified RNAs within a sample and assess their abundance via HTG EdgeSeq. The level of enrichment can be measured by assessment of the relative levels of modified and unmodified RNA spike in controls. Overall, these results demonstrate the ability of the disclosed methods to differentiate changes in modification between treated and untreated samples. Further, the results also show that the disclosed methods can distinguish modified RNA and modified DNA, reducing false-positive signals.

### Example 3

### A Reproducibility Study of Enrichment and Detection of Modified RNAs.

This example describes methods used to enrich for and isolate modified RNAs within a cell lysate sample, followed by detection using a nuclease-protection mediated sequencing assay. Reproducibility of the described methods between replicates, days, operators, antibodies, and sample input is characterized herein.

Two experiments were performed. In the first, a single sample was chosen, and enrichment of N⁶-methyadenoside modified (m⁶A-modified) RNAs from this sample was followed by assessment of the RNAs using a detection away, such as a nuclease-protection mediated sequencing assay (e.g., an HTG EdgeSeq assay designed to measure the whole transcriptome). The sample was run in triplicate by three different operators on different days and the results compared. To determine whether reproducibility was dependent on the antibody used, a second antibody was used for enrichment of a fourth set of triplicate samples. In the second experiment, a single sample was utilized but was run in triplicate in a four-point sample input titration (for cell lysates) or a five-point sample input titration (for isolated RNA). Considered together, these two experiments demonstrate the reproducibility of the described methods, across replicates, operators, antibodies, and a range of sample input.

Cell line lysates were treated with DNase as described in Example 2 prior to being enriched for modified RNAs. RNA was isolated using TRIzol and treated with DNase as part of the isolation process.

Immunoprecipitation reactions comprised:
Sample:
   Either cell lysate:
      HEK293T cells in a lysis buffer at 110,000 cells per sample (for repeatability) or 110K, 55K; 28K, 14K (K=thousand) cells per sample or isolated RNA:
      500, 250,125, 62.5, or 31.25 ng RNA isolated from HEK293T cells,
33 attomoles of modified and unmodified IVTs (as described in previous examples),
1 μl RNasin to prevent RNase activity,
an immunoprecipitation buffer,
20 μl of protein G beads (Thermo) pre-conjugated with N⁶-methyladenosine antibody from New England Biolabs (#E1610S) or Cell Signaling Technologies (mAb #56593).

Immunoprecipitation was performed as described in Example 1. Washing was performed using three buffers (Buffer 1 is the same composition as the immunoprecipitation reaction; Buffer 2: 50 mM NaCl, 0.1% IGEPAL CA-630, 10 mM Tris pH 7.4; Buffer 3: 500 mM NaCl, 0.1% IGEPAL CA-630, 10 mM Tris pH 7.4). One wash in Buffer 1 was followed by two washes in Buffer 2 and two washes in Buffer 3. Washes were performed at room temperature. RNAs were eluted from the beads via addition of a lysis buffer followed by heating to 95° C. for 15 minutes. The resulting enriched RNA samples were then assessed using the HTG Transcriptome Panel, an HTG EdgeSeq assay that comprises over 19,000 NPPFs designed to the human transcriptome (available from HTG Molecular Diagnostics, Inc., of Tucson, Arizona). For all cell lines, the DNase-treated parent sample, without enrichment for modified RNAs, was also assessed using the same HTG EdgeSeq assay. All samples and pulldowns were run in triplicate.

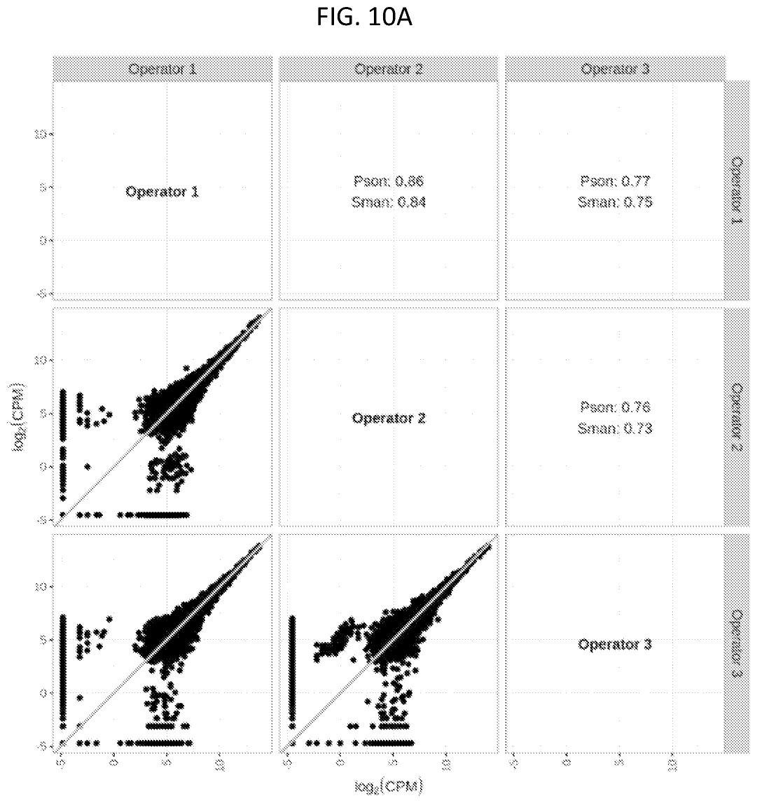

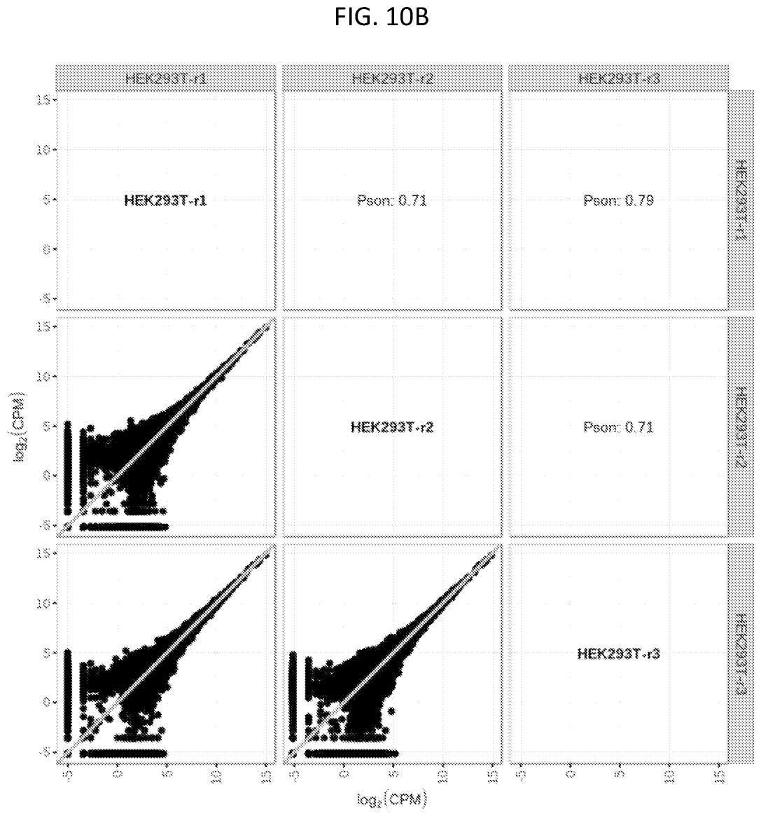

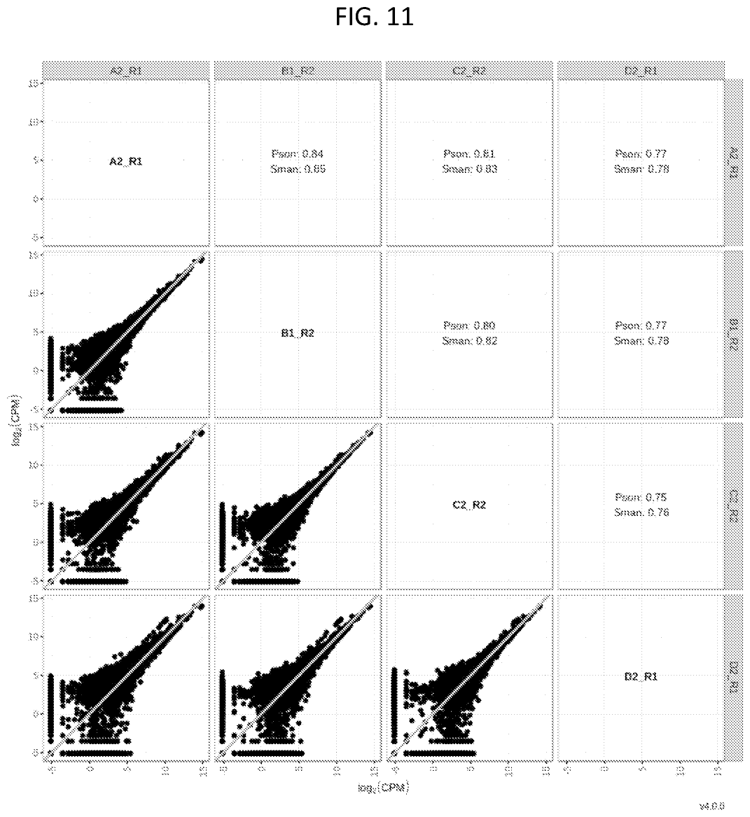

Data were assessed for each experiment for enrichment specificity and correlation of replicates. Raw data were standardized using a log₂CPM calculation as described in previous examples, then Pearson correlations were calculated for each replicate pair. FIG. 10A shows a representative set of replicates, each one run by a different operator on a different day. The median Pearson correlation coefficient for these three samples was 0.65 using all data and 0.77 when filtering out the lowest signals (filtered data shown). The lowest enrichment score for this entire experiment was 455×, indicating that the data are not only highly repeatable, but that true signal is the bulk of what is measured. A similar experiment was performed using a second anti-N⁶-methyladenosine antibody, in this case using only a single operator. Replicate correlation for this experiment, shown in FIG. 10B was similarly robust (median Pearson correlation coefficient of 0.71). These results show that the described methods are reproducible and robust to changes in operator, day, and antibody used for the immunoprecipitation.

FIG. 11 shows a correlation scatterplot with Pearson correlation coefficients for a representative set of replicates from a range of sample input (110,000 cells to 14,000 cells per sample) added to the immunoprecipitation reaction. The correlation between representative samples from this sample input range are shown; the median Pearson correlation is 0.79, with only a small decrease between the highest and lowest sample inputs. A similar result was seen when using isolated RNA as the input (not shown), with some loss of correlation due to increased noise at the low signal end as the input drops below 50 ng. This is a surprisingly good result for such a low input sample.

These results demonstrate the reproducibility of the described methods to enrich for modified RNAs within samples. Excellent repeatability was observed across multiple operators, days, sample input amounts, and also using two different antibodies, effectively demonstrating the robustness of this process. Establishing reproducibility and robustness can be important to using the described techniques in experiments designed for discovery purposes, as will be described in the next two examples.

### Example 4

Enrichment and Detection of Modified RNAs from Cells Treated with an Agent.

This example describes methods used to enrich for and isolate modified RNAs within a pair of cell lysate samples; the cells in the samples having been treated with an agent or not treated, or treated with an agent and a control. Comparison of the results from both samples demonstrates the ability to identify RNAs modified by the agent, as well as to measure expression-level RNA changes resulting from treatment with an agent.

The agent used in this Example is the application of a heat shock treatment to a flask of HEK293T cells (the control being a non-heat-shock treated flask of otherwise identical cells). For the heat shock treatment, cells were incubated at 42° C. for 1 hour in a diy bath followed by 1 hour of recovery at 37° C. in a 5% CO₂ incubator. Cells were grown, treated or not treated, collected, and lysed in a lysis buffer.

Cell line lysates were treated with DNase as described in Example 2 prior to being enriched for modified RNAs. Immunoprecipitation reactions comprised:
110,000 HEK293T cells in a lysis buffer,
33 attomoles of modified and unmodified IVTs (as described in previous examples), 1 μl RNasin to prevent RNase activity,
an immunoprecipitation buffer,
20 μl of protein G beads (Thermo) pre-conjugated with anti-N⁶-methyladenosine antibody from Cell Signaling Technologies

Immunoprecipitation and washing of the beads was performed as described in Example 3. RNAs were eluted from the beads via addition of a lysis buffer followed by heating to 95° C. for 15 minutes. The resulting enriched RNA samples were then assessed using the HTG Transcriptome Panel, an HTG EdgeSeq assay that comprises over 19,000 NPPFs designed to the human transcriptome. For all cell lines, the DNase-treated parent sample, without enrichment, was also assessed using the same HTG EdgeSeq assay. All samples and pulldowns were run in triplicate. Data were assessed for each experiment for signal-to-noise or specificity of the enrichment, correlation of pulldown sample data, correlation of parent sample data, and then for expression level and modification level changes between agent-treated and control cells.

Signal to noise or specificity of the antibody reaction was calculated by dividing the average signal of the two “signal” or modified IVTs by the average signal of the two “noise” or unmodified IVTs within the pulldown reactions. An adjusted specificity score was calculated by dividing the ratio of modified to unmodified IVT signals within the parent sample (as these samples should have no enrichment of the modified IVTs, and thus are expected to have a theoretical 1:1 ratio). Specificity scores ranged from 225-354; the antibody itself is expected by the vendor to provide at least a 20× enrichment.

Genomic DNA probes measured no signals greater than background in the parent cell lines, demonstrating the effectiveness of the DNase treatment, as discussed in Example 2 (data not shown).

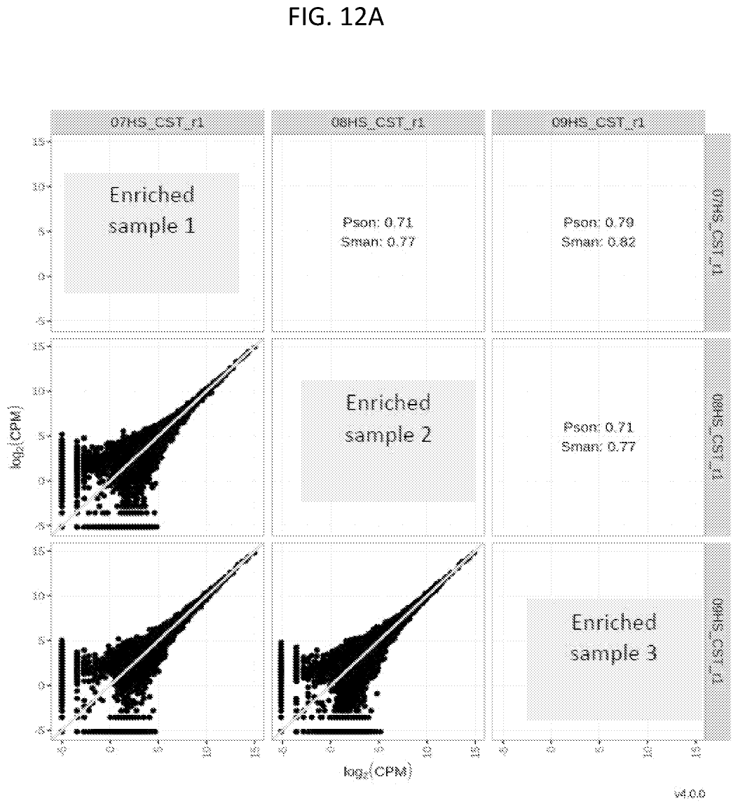

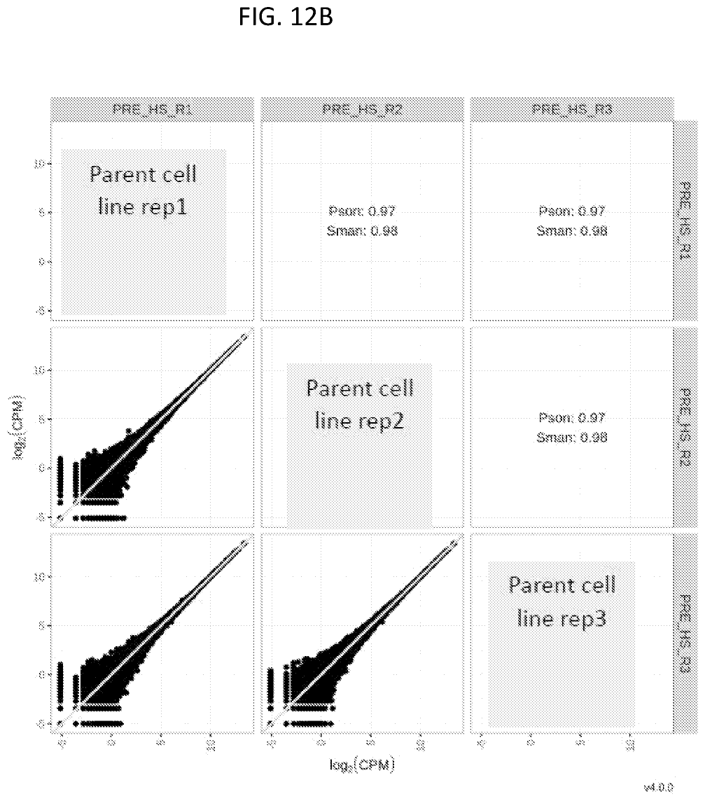

Second, the repeatability of pulldown and parent sample data was assessed from raw data using a standard correlation coefficient calculation. A representative example of pulldown samples is shown in FIG. 12A, and one for parent samples is shown in FIG. 12B; the Pearson correlations for the parent samples were 0.97, and those for the pulldown samples ranged from 0.71-0.79. The examples shown in this figure are for heat shock-treated cells.

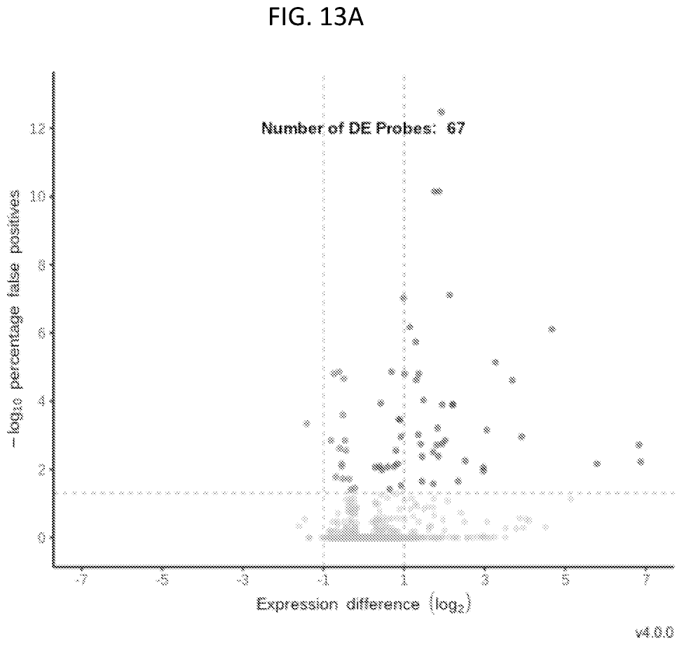

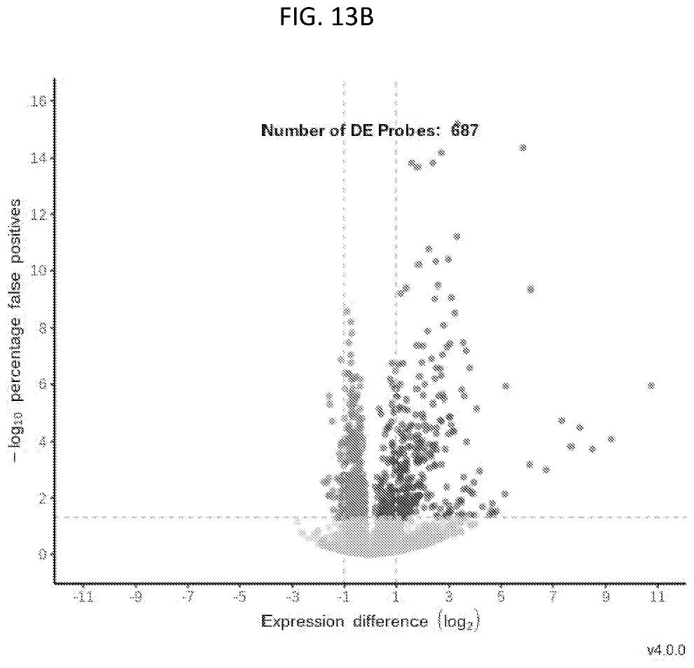

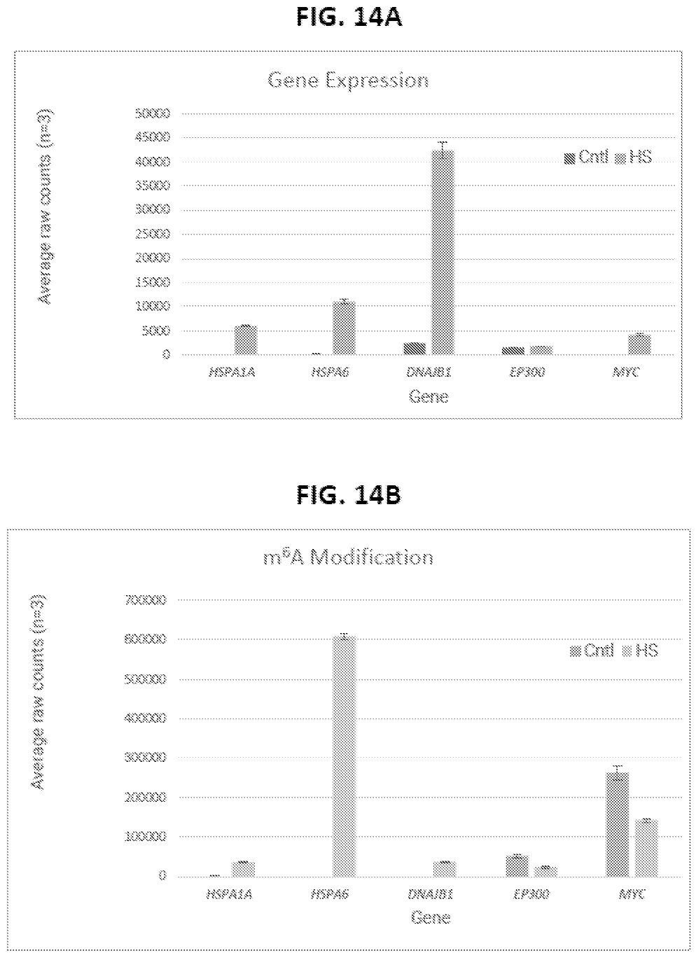

Finally, the effect of heat shock treatment on both expression and m⁶A modification levels was assessed. For this assessment, each parent or pulldown dataset was compared to its control in a differential expression analysis, generated from log₂CPM data using the edgeR package in R. FIG. 13A shows a volcano plot of the differentially expressed genes within the heat shock agent/control expression data. Of the ˜20,000 RNAs measured by the assay, only 67 were significantly up- or down-regulated. The genes so regulated are largely part of the heat shock response pathway and include HSPA6, DNAJB1, and the genes that encode Hsp1, a core heat shock response protein complex. A similar analysis of the pulldown data, shown in FIG. 13B, shows that 687 genes have changes to m⁶A modification level following exposure to heat shock. From these data, three conclusions may be made. One, heat shock stress for this short duration strongly upregulates the heat shock pathway, but appears to affect few other RNAs. Second, there is a very strong upregulation of m⁶A modification of several heat-shock related genes during this stress. Third, expression and m⁶A modification are independent of one another; this is most clearly seen in FIGS. 14A and 14B, when examining the changes affecting HSP6, DNAJB1, and MYC. DNAJB1 is strongly upregulated at the expression level (FIG. 14A) but m⁶A modification (FIG. 14B) is only somewhat upregulated, while MYC expression increases but m⁶A modification is downregulated. HSPA6 is strongly upregulated both at expression and m⁶A modification levels. This demonstrates the ability of the techniques described to examine and characterize both levels of regulation (e.g., expression and m⁶A modification) in response to an agent (such as heat shock or other stress). These results demonstrate the ability of the disclosed methods to enrich for modified RNAs within a sample, assess their abundance via HTG EdgeSeq, and compare the results from agent-treated and agent-untreated samples to identify regulation at the modification and expression levels of RNAs within the sample. The results demonstrate repeatable and significant changes to both m⁶A modification and expression level of specific RNAs in response to an agent.

### Example 5

Enrichment, Detection, and Comparison of Modified RNAs from Cells Treated with Agents or a Control.

This example describes methods used to enrich for and isolate modified RNAs within cell lysate samples; the cells in the samples having been treated with an agent (such as a small molecule) or treated with a control substance. Comparison of the results from both samples demonstrates the ability to identify RNAs modified by the agent, as well as to measure expression-level RNA changes resulting from treatment with an agent. Collectively, the data may also be used to determine similarities and differences between a group of different agents.

The agents used in this Example are a six small molecule compounds [see Table 5] in DMSO, or with DMSO alone. PC3 cells were grown, treated with agent(s) (10 nM for 24 hours) and DMSO controls, then collected, and lysed in a lysis buffer.

Cell line lysates were treated with DNase as described in Example 2 prior to being enriched for modified RNAs. Immunoprecipitation reactions comprised:
44,000 PC3 cells in a lysis buffer,
13 attomoles of modified and unmodified IVTs (as described in previous examples),
1 μl RNasin to prevent RNase activity,
an immunoprecipitation buffer,
20 μl of protein G beads (Thermo) pre-conjugated with N6-methyladenosine antibody from Cell Signaling Technologies

Immunoprecipitation and washing of the beads was performed as described in Example 3, except for reduction of the wash steps to one wash with Buffer 1, one with Buffer 2, and one with Buffer 3, all at room temperature. RNAs were eluted from the beads via addition of a lysis buffer followed by heating to 95° C. for 15 minutes. The resulting enriched RNA samples were then assessed using the HTG Transcriptome Panel, an HTG EdgeSeq assay that comprises over 19,000 NPPFs designed to the human transcriptome. Additionally, for each agent treatment or control, a DNase-treated parent sample, without enrichment, was also assessed using the same HTG EdgeSeq assay. All samples and pulldowns were run in triplicate. Data were assessed for each experiment for signal-to-noise or specificity of the enrichment, correlation of pulldown sample data, correlation of parent sample data, and then for expression level and modification level changes between agent-treated and control cells and between agent-treated cells.

Signal to noise or specificity of the antibody reaction was calculated as described in previous examples. A median enrichment of 83× was observed for these samples. Genomic DNA probes measured no signals greater than background in the parent cell lines, demonstrating the effectiveness of the DNase treatment, as discussed in Example 2 (data not shown). Additionally, repeatability was assessed for parent and pulldown samples; the median correlation for parents was 0.98 and for pulldowns was 0.69.

The effect of agent treatment on both RNA expression and m6A modification levels was assessed. Two methods were used for comparison. First, each agent-treated cell line parent or pulldown dataset was compared to its control in a differential expression analysis, generated from log 2CPM data using the DESeq2 package in R. Table 5 below shows the number of significantly differentially expressed or differentially modified genes from each agent treatment. An adjusted p-value cutoff of 0.05 (less than or equal to) was use as the significance threshold.

TABLE 5
Number of Differentially Expressed or Modified Genes
Significantly differentially modified or expressed
RNAs measured (compound compared to DMSO control).

| Compound | Significantly differentially | Significantly differentially |
|---|---|---|
| name | modified RNAs | expressed RNAs |
| HTW0741 | 12 | 593 |
| HTW0186 | 773 | 3429 |
| HTW0544 | 201 | 813 |
| HTW0744 | 70 | 365 |
| HTW0457 | 365 | 4155 |
| HTW0902 | 1351 | 7710 |

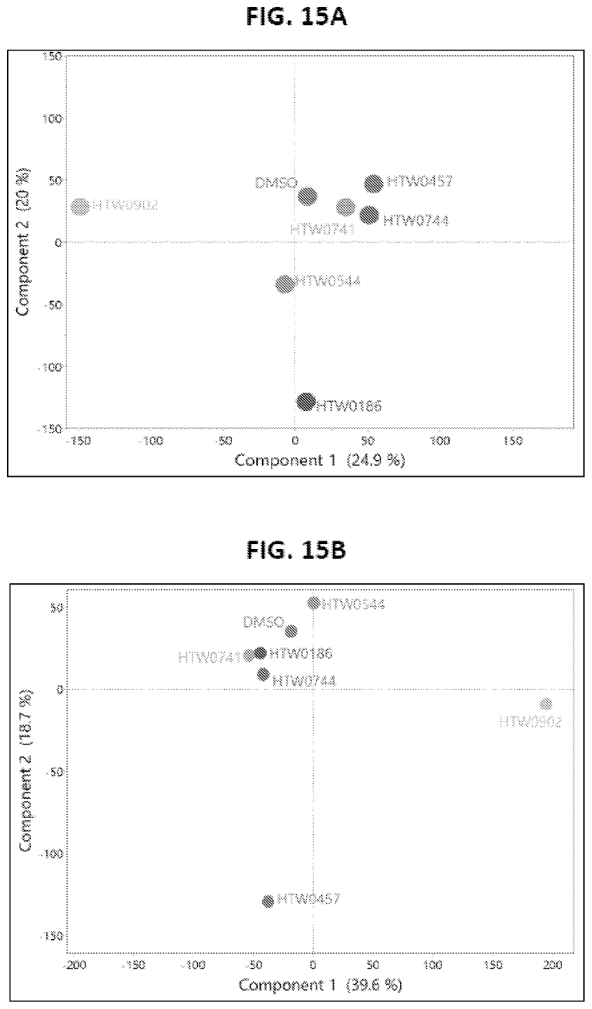

This set of agents was designed to inhibit the same protein target, so a point of interest was the relative similarity or differences of the modification profiles. A second comparison was performed using log₂CPM standardized data for each modification or expression profile using a principal component analysis (PCA). Two PCA plots are shown in FIGS. 15A and 15B, with FIG. 15A showing the m⁶A modifications that were measured and FIG. 15B showing the measured RNA expression profiles. Interestingly, there is a large range, with some compounds clustered close to the DMSO control, and others further away. These comparison analyses demonstrate that differences between m⁶A modification profiles may be reproducibly measured, and the data may next be used to understand the differences in biological consequences between treatments with these different compounds.

These results demonstrate the ability of the disclosed methods to enrich for modified RNAs within a sample, assess their abundance via HTG EdgeSeq, and compare the results between a set of treated samples. The results demonstrate repeatable and significant changes to both m6A modification and expression level of specific RNAs in response to an agent. Further, the results of this and the previous example demonstrate that the definition of “agent” is broad and may encompass at least stress or disease conditions, or a compound or drug molecule.

In view of the many possible embodiments to which the principles of the disclosed invention may be applied, it should be recognized that the illustrated embodiments are only examples of the disclosure and should not be taken as limiting the scope of the disclosure. Rather, the scope of the invention is defined by the following claims. I therefore claim as my invention all that comes within the scope and spirit of these claims.

1. A method of determining a sequence of a modified nucleic acid molecule in a sample, comprising:
contacting the sample with an antibody that specifically binds to a modified nucleic acid molecule under conditions that allow the antibody to bind to the modified nucleic acid molecule in the sample, thereby forming an antibody-nucleic acid conjugate;
separating the antibody-nucleic acid conjugate from unbound, unmodified nucleic acids and optionally separating the antibody from the modified nucleic acid molecule, thereby generating purified modified nucleic acid molecule;
contacting the purified modified nucleic acid molecule with at least one nuclease protection probe comprising a flanking sequence (NPPF) under conditions sufficient for the NPPF to specifically hybridize to the purified modified nucleic acid molecule,
wherein the NPPF comprises:
a 5′-end and a 3′-end,
a sequence complementary to a region of the modified nucleic acid molecule, permitting specific hybridize between the NPPF and the modified nucleic acid molecule,
a flanking sequence located 5′, 3′, or both, to the sequence complementary to the modified nucleic acid molecule, wherein the flanking sequence comprises at least 12 contiguous nucleotides not found in a nucleic acid molecule present in the sample, providing a universal amplification sequence, and wherein the flanking sequence is complementary to at least a portion of an amplification primer;
contacting the purified modified nucleic acid molecule with a nucleic acid molecule comprising a sequence complementary to the flanking sequence (CFS) under conditions sufficient for the flanking sequence to specifically hybridize to the CFS;
contacting the purified modified nucleic acid molecule with a nuclease specific for single-stranded nucleic acid molecules under conditions sufficient to remove unbound nucleic acid molecules, thereby generating a digested sample comprising NPPFs hybridized to the purified modified nucleic acid molecule and CFSs;
amplifying NPPFs in the digested sample with the amplification primer, thereby generating NPPF amplicons, wherein at least one amplification primer comprises a region that is complementary to the flanking sequence of the NPPF; and
sequencing at least a portion of the NPPF amplicons, thereby determining the sequence of the modified nucleic acid molecule in the sample.

2.-5. (canceled)

6. The method of claim 1, wherein the NPPF comprises a flanking sequence at the 5′-end and the 3′-end, wherein the flanking sequence at the 5′-end differs from the flanking sequence at the 3′-end.

7. The method of claim 1, wherein the at least one amplification primer further comprises a sequence that permits attachment of an experimental tag or sequencing adapter to the NPPF amplicon during the amplification step.

8. The method of claim 1, wherein the flanking sequence further comprises an experimental tag, sequencing adapter, or both.

9.-11. (canceled)

12. The method of claim 1, wherein the modified nucleic acid molecule is fixed, cross-linked, or insoluble.

13. (canceled)

14. The method of claim 1, wherein the NPPF is a DNA and the nuclease comprises an exonuclease, an endonuclease, or a combination thereof.

15.-16. (canceled)

17. The method of claim 1, wherein the method sequences at least 1000 different modified nucleic acid molecules, and wherein the sample is contacted with at least 10,000 different NPPFs, each NPPF specific for a different modified nucleic acid molecule.

18. The method of claim 1, wherein the method is performed on a plurality of samples and at least 1000 different modified nucleic acid molecules are detected in each of the plurality of samples.

19. The method of claim 1, wherein at least one NPPF is specific for a modified miRNA molecule and at least one NPPF is specific for a modified mRNA molecule.

20.-22. (canceled)

23. The method of claim 1, further comprising:
comparing an NPPF sequence obtained to a reference sequence database; and
determining a number of each identified NPPF sequences.

24.-25. (canceled)

26. The method of claim 1, wherein the modified nucleic acid molecule is modified RNA.

27. (canceled)

28. The method of claim 1, further comprising contacting the sample with a DNAase prior to contacting the sample with the antibody.

29. The method of claim 1, wherein the at least one modified nucleic acid molecule is modified DNA.

30. The method of claim 1, wherein the at least one modified nucleic acid molecule comprises one or more N⁶-methyladenosine (m⁶A) bases, and the antibody comprises an anti-N⁶-methyladenosine antibody.

31. The method of claim 1, wherein the antibody contacted with the sample is conjugated to a solid support, and wherein optionally separating the antibody-nucleic acid conjugate from unbound, unmodified nucleic acids comprises washing the solid support comprising the antibody-nucleic acid conjugate.

32.-33. (canceled)

34. The method of claim 1, wherein the sample comprises a cell lysate.

35.-36. (canceled)

37. The method of claim 1, further comprising:
contacting the sample with an agent prior to contacting the sample with the antibody, wherein the method determines if the agent generates an on target modified nucleic acid molecule in the sample, an off-target modified nucleic acid molecule in the sample, or both.

38. The method of claim 37, wherein the agent comprises one or more mutagens, microbes, therapeutic agents, heat shock, or combinations thereof.

39. The method of claim 1, wherein the NPPF is within at least two nucleotides or ribonucleotides of the modification in the modified nucleic acid molecule.

40. The method of claim 1, further comprising determining a sequence of modified nucleic acid molecules and unmodified nucleic acids in a separate portion of the sample, comprising:
contacting the separate portion of the sample with at least two NPPFs under conditions sufficient for at least one NPPF to specifically hybridize to a modified nucleic acid molecule in the separate portion of the sample and for another at least one NPPF to specifically hybridize to an unmodified nucleic acid molecule in the separate portion of the sample,
wherein each NPPF comprises:
a 5′-end and a 3′-end,
a sequence complementary to a region of the modified nucleic acid molecule or the unmodified nucleic acid molecule, permitting specific hybridization between the NPPF and the modified nucleic acid molecule or the unmodified nucleic acid molecule, respectively,
a flanking sequence located 5′, 3′, or both, to the sequence complementary to the modified or unmodified nucleic acid molecule, wherein the flanking sequence comprises at least 12 contiguous nucleotides not found in a nucleic acid molecule present in the sample, providing a universal amplification sequence, and wherein the flanking sequence is complementary to at least a portion of an amplification primer;
contacting the separate portion of the sample with a nucleic acid molecule comprising a sequence complementary to the flanking sequence (CFS) under conditions sufficient for the flanking sequence to specifically hybridize to the CFS;
contacting the separate portion of the sample with a nuclease specific for single-stranded nucleic acid molecules under conditions sufficient to remove unbound nucleic acid molecules, thereby generating a digested separate portion of the sample comprising NPPFs hybridized to the modified and unmodified nucleic acid molecules and CFSs;
amplifying NPPFs in the digested separate portion of the sample with the amplification primer, thereby generating NPPF amplicons, wherein at least one amplification primer comprises a region that is complementary to the flanking sequence of the NPPF; and
sequencing at least a portion of the NPPF amplicons, thereby determining the sequence of the modified and unmodified nucleic acid molecules in the separate portion of the sample.


SEQUENCE LISTING (4 sequences)

SEQ ID NO: 1 - DNA, 18 bp, synthetic construct
  source 1..18: mol_type = other DNA
ttgcccgatgcaatgaga

SEQ ID NO: 2 - DNA, 21 bp, synthetic construct
  source 1..21: mol_type = other DNA
cgcctgaagaagctgagataa

SEQ ID NO: 3 - DNA, 23 bp, synthetic construct
  source 1..23: mol_type = other DNA
catgattgtgacctcggtatctc

SEQ ID NO: 4 - DNA, 21 bp, synthetic construct
  source 1..21: mol_type = other DNA
ggttcaagggtgaggctattt